(19) United States
(12) Patent Application Publication
(10) Pub. No.: US 2026/0004901 A1
(43) Pub. Date: Jan. 1, 2026

(54) FUNCTIONAL BIOLOGICAL MODELING SYSTEM

(71) Applicant: Tempus AI, Inc., Chicago, IL (US)

(72) Inventors: Christopher Shane Colley, Naperville, IL (US); Eric P. Lefkofsky, Chicago, IL (US); Amy Kate Sasser, Doylestown, PA (US)

(21) Appl. No.: 19/255,990

(22) Filed: Jun. 30, 2025

Related U.S. Application Data
(60) Provisional application No. 63/665,923, filed on Jun. 28, 2024.

(51) Int. Cl. G16H 10/60; G16H 15/00; G16H 20/10; G16H 50/20; G16H 50/50

(52) U.S. Cl. CPC G16H 10/60; G16H 15/00; G16H 20/10; G16H 50/20; G16H 50/50

(57) ABSTRACT

Methods, systems, and software are provided for predicting care pathway options for a medical condition in a test subject. In one implementation, a method includes retrieving a set of characteristics of the test subject from an electronic medical record for the test subject, retrieving data from a system modeling human tissue, and providing information comprising the set of characteristics from the electronic medical record and the data from the system modeling human tissue to an artificial intelligence (AI) component to receive as output from the AI component one or more care pathways for the medical condition.

### CROSS REFERENCE TO RELATED APPLICATIONS

This application claims priority to U.S. Provisional Patent Application No. 63/665,923, entitled “FUNCTIONAL BIOLOGICAL MODELING SYSTEM,” filed Jun. 28, 2024, which is hereby incorporated by reference.

### FIELD OF THE INVENTION

The present disclosure relates generally to the use of electronic medical data and tissue modeling to provide clinical support for personalized therapy.

### BACKGROUND

Precision medicine is the practice of tailoring therapy to the unique biology of a subject using, for example, genomic, epigenetic, and/or transcriptomic profiles from the subject to inform treatment of various disorders. Personalized oncology, for example, was borne out of many observations that different patients diagnosed with the same type of cancer, e.g., breast cancer, responded very differently to common treatment regimens. Over time, researchers have identified genomic, epigenetic, and transcriptomic markers that improve predictions as to how an individual cancer will respond to a particular treatment modality.

There is growing evidence that cancer patients who receive therapy guided by their genetics have better outcomes. For example, studies have shown that targeted therapies result in significantly improved progression-free cancer survival. See, e.g., Radovich et al., Oncotarget, 7 (35): 56491-500 (2016). Similarly, reports from the IMPACT trial—a large (n=1307) retrospective analysis of consecutive, prospectively molecularly profiled patients with advanced cancer who participated in a large, personalized medicine trial—indicate that patients receiving targeted therapies matched to their tumor biology had a response rate of 16.2%, as opposed to a response rate of 5.2% for patients receiving non-matched therapy. See, Tsimberidou et al., ASCO 2018, Abstract LBA2553 (2018).

In fact, therapy targeted to specific genomic alterations is already the standard of care in several tumor types, e.g., as suggested in the National Comprehensive Cancer Network (NCCN) guidelines for melanoma, colorectal cancer, and non-small cell lung cancer. In practice, implementation of these targeted therapies requires determining the status of the diagnostic marker in each eligible cancer patient. While these personalized strategies improve therapeutic outcomes, there is still a range of responses even within the patient population with well-associated biological markers. It is believed that at least some of this variability is due to other differences in the biology between patients.

One strategy to account for the diverse and personalized biology of cancer is through testing of patient-derived cell cultures, such as tumor organoids. Such patient-derived tumor organoids can be used to model cancer growth and estimate the effectiveness of different therapies as applied to the subject's cancer.

The information disclosed in this Background section is only for enhancement of understanding of the general background of the invention and should not be taken as an acknowledgement or any form of suggestion that this information forms the prior art already known to a person skilled in the art.

### SUMMARY

Given the above background, there is a need in the art for improved methods and systems for supporting clinical decisions in precision medicine. In particular, there is a need for improved methods and systems for modeling a patient's unique biology to identify improved personalized care pathways. The present disclosure solves this and other needs in the art, at least in part, by providing methods and systems for using artificial intelligence to combine analysis from electronic health records with tissue modeling data to identify personalized care pathways for treating a disorder.

In one aspect, the disclosure provides a system that recreates aspects of human biology, including tissue function or disease and combines the data from the recreation with data from a subject's EHR, e.g., clinical notes, test results, medical images, etc., to predict care pathway options using a computer model. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

Example methods for tissue slice culturing are described in Kenerson et al., “Protocol for tissue slice cultures from human solid tumors to study therapeutic response,” STAR Protoc. 2021 Jun. 2; 2 (2): 100574, the content of which is incorporated herein by reference in its entirety.

In some embodiments, predictions from the method are then tested on recreated human tissue, e.g., using cell lines, organoids, tissue slice cultures, PDX models, and/or microfluidics, etc. In some embodiments, it is determined, based on the prediction testing, whether the prediction is accurate or should be modified. In some embodiments, a prediction is provided to a clinician/patient/healthcare system.

In one aspect, the disclosure provides a system that recreates aspects of human biology, including tissue function or disease, tests different treatments on the recreated human tissue, using a model combining and analyzing the data received from the testing and a subject's health information, and using a second model predicting which tested treatment to match to the subject. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

In some embodiments, predictions from the method are then tested on recreated human tissue, e.g., using cell lines, organoids, tissue slice cultures, PDX models, microfluidics, etc. In some embodiments, it is determined, based on the prediction testing, whether the prediction is accurate or should be modified. In some embodiments, a prediction is provided to a clinician/patient/healthcare system.

In one aspect, the disclosure provides a system integrated in a healthcare network that deploys models for use by clinicians to compare and analyze subject healthcare data in combination with data from human tissue recreations to provide insights to clinicians on care options for one or more subjects. In some embodiments, the system facilitates provisioning of similar subjects into cohorts for analysis. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

In one aspect, the disclosure provides a system for querying biological data that ingests data from recreated human biology, sequencing data, imaging data and health records, uses models to combine and align all of the data, uses a computer model to analyze the data and permits a user to query the data based on different parameters and features. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

Accordingly, in some embodiments, the disclosure provides methods, systems, and computer readable media for predicting care pathway options for a medical condition in a test subject. The method includes retrieving a set of characteristics of the test subject from an electronic medical record for the test subject, retrieving data from a system modeling human tissue, and providing information comprising the set of characteristics from the electronic medical record and the data from the system modeling human tissue to an artificial intelligence (AI) component to receive as output from the AI component one or more care pathways for the medical condition.

In one aspect, the disclosure provides a system for identifying patient specific treatments/therapies where a computer model ingests a subject's EHR, including clinical, molecular, and/or imaging data, and analyzes the data to provide a prediction on which treatment(s)/therapy(ies) are best for the subject. Cells and/or tissues are then used to recreate the subject's response to treatment(s)/therapy(ies) and the results from each are fed into a further model to compare the results of each and identify the best/next treatment option. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

In some embodiments, the system simulates experimental results, using past experimental results. In some embodiments, this facilitates selection of therapeutic dosages that can be tested on tissue recreations. In some embodiments, predictions from the method are then tested on recreated human tissue, e.g., using cell lines, organoids, tissue slice cultures, PDX models, microfluidics, etc. In some embodiments, it is determined, based on the prediction testing, whether the prediction is accurate or should be modified. In some embodiments, a prediction is provided to a clinician/patient/healthcare system.

Accordingly, in some embodiments, the disclosure provides a method for predicting care pathway options for a test subject. The method includes providing information from an electronic medical record for the test subject to a first artificial intelligence (AI) component to determine a set of therapies for a medical condition. The method then includes testing the set of therapies on a system modeling human tissue (e.g., a system modeling tissue afflicted with the medical condition) to receive modeling data as output from the testing. The method also includes providing second information comprising the modeling data to a second artificial intelligence (AI) component to receive as output from the AI component one or more care pathways for the medical condition.

In one aspect, the disclosure provides a system for monitoring drug interactions or potential toxicities, where a model ingests on a recurring basis sequencing data, imaging data, and/or health records, along with data from recreated human tissue, uses a computer model to analyze the data, e.g., based on treatment responses under different conditions/in different subjects, segments the data based on treatment response and updates interactions as new data is received. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

In one aspect, the disclosure provides a system for monitoring drug interactions in a subject where the subject's disease is replicated using human tissue, using a model to combine the data from the replicated human tissue and the subject's health records, analyzing the combined data to compare reactions in the replicated human tissue with reactions presented in the health records. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

In one aspect, the disclosure provides a system for diagnosing a disease by ingesting a subject's EHR, using human tissue to model the subject's clinical responses shown in the EHR, combining that data to identify the subject's unknown disease or correct a misidentified diagnosis. In some embodiments, modeling of the subject's clinical responses is achieved through culture modeling, e.g., using cell lines, organoids, tissue slice cultures, PDX models, microfluidics, etc. In some embodiments, modeling of the subject's clinical responses is achieved through in silico modeling and/or data extrapolation. In some embodiments, the computer model is a large language model, large imaging model, or other generative AI model, e.g., that perform similar functions to large language models.

In some embodiments, therapies to be tested on the recreations are selected from a list of therapies to which other patients in a database responded (e.g., saw significant health improvements and/or slowed disease progression), wherein the database patients have similarities to the subject's molecular and/or clinical characteristics. In some embodiments, the subject's molecular and/or clinical characteristics do not match with any conventional/approved treatment regimen. In some embodiments, the subject's molecular and/or clinical characteristics do match with at least one conventional/approved treatment regimen, but the subject has already tried the therapies without significant improvement to the patient's health.

Accordingly, in some embodiments, the disclosure provides a method for evaluating a medical condition in a test subject. The method includes providing information from an electronic medical record for the test subject to a model, which could be an artificial intelligence and/or machine learning model, to receive as output from the model a set of tests for modeling a tissue. The method also includes performing the set of tests on a system modeling human tissue to receive modeling data as output from the testing. The method also includes providing second information comprising the modeling data to an artificial intelligence (AI) component to receive as output from the AI component an analysis of the medical condition in the test subject.

Accordingly, in some embodiments, the disclosure provides a method for identifying a new care pathway for a medical condition. The method includes retrieving, for each respective subject in a plurality of subjects, a corresponding set of characteristics of the respective subject from a corresponding electronic medical record for the respective subject. The method also includes retrieving data from a system modeling human tissue associated with a medical condition. The method includes providing information comprising (i) the corresponding set of characteristics from the corresponding electronic medical record for each respective subject in the plurality of subjects and the data from the system modeling human tissue afflicted with a medical condition to an artificial intelligence (AI) component to receive as output from the AI component a target or therapy representing a new care pathway for the medical condition. In some embodiments, recreation of aspects of human biology are achieved through culture modeling, e.g., using cell lines, organoids, tissue slice cultures, PDX models, microfluidics, etc. In some embodiments, recreation of aspects of human biology are achieved through in silico modeling and/or data extrapolation. In some embodiments, the computer model is a large language model, large imaging model, or other generative AI model, e.g., that perform similar functions to large language models.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 illustrates a block diagram of an example computing device for identifying care pathway options for a medical condition in a test subject, in accordance with some embodiments of the present disclosure.

FIG. 2A illustrates an example workflow for generating a clinical report based on information generated from analysis of one or more patient specimens, in accordance with some embodiments of the present disclosure.

FIG. 2B illustrates an example of a distributed diagnostic environment for collecting and evaluating patient data for the purpose of precision medicine, in accordance with some embodiments of the present disclosure.

FIG. 3 provides an example flow chart of processes and features for liquid biopsy sample collection and analysis for use in precision medicine, in accordance with some embodiments of the present disclosure.

FIG. 4 illustrates an example nucleic acid sequence analysis for bioinformatic analysis, e.g., for precision medicine, in accordance with some embodiments of the present disclosure.

FIGS. 5A, 5B, 5C, 5D, 5E, 5F, and 5G collectively provide a flow chart of processes and features for predicting care pathway options for a medical condition in a test subject, in which dashed boxes represent optional portions of the method, in accordance with some embodiments of the present disclosure.

FIGS. 6A, 6B, 6C, 6D, 6E, 6F, 6G, and 6H collectively provide a flow chart of processes and features for predicting care pathway options for a medical condition in a test subject, in which dashed boxes represent optional portions of the method, in accordance with some embodiments of the present disclosure.

FIGS. 7A, 7B, 7C, 7D, 7E, 7F, 7G, and 7H collectively provide a flow chart of processes and features for evaluating a medical condition in a test subject, in which dashed boxes represent optional portions of the method, in accordance with some embodiments of the present disclosure.

FIGS. 8A, 8B, 8C, 8D, 8E, 8F, and 8G, collectively provide a flow chart of processes and features for identifying a new care pathway for a medical condition, in which dashed boxes represent optional portions of the method, in accordance with some embodiments of the present disclosure.

FIG. 9 illustrates an example process for identifying care pathways by matching human samples with organoid samples, in accordance with some embodiments of the present disclosure.

Like reference numerals refer to corresponding parts throughout the several views of the drawings.

### DETAILED DESCRIPTION

### Definitions

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the terms “control,” “control sample,” “reference,” “reference sample,” “normal,” and “normal sample” describe a sample from a non-diseased tissue. In some embodiments, such a sample is from a subject that does not have a particular condition (e.g., cancer). In other embodiments, such a sample is an internal control from a subject, e.g., who may or may not have the particular disease (e.g., cancer), but is from a healthy tissue of the subject. For example, where a liquid or solid tumor sample is obtained from a subject with cancer, an internal control sample may be obtained from a healthy tissue of the subject, e.g., a white blood cell sample from a subject without a blood cancer or a solid germline tissue sample from the subject. Accordingly, a reference sample can be obtained from the subject or from a database, e.g., from a second subject who does not have the particular disease (e.g., cancer).

As used herein, the term “care pathway” refers to all or a portion of a therapeutic strategy for treating a disorder in a subject. In some embodiments, one or more therapies making up a care pathway may be selected for a patient in a series or in combination (for example, overlapping treatments), e.g., based on the patient's diagnosis and/or prognosis as informed by the methods disclosed herein.

In some embodiments, a care pathway is a structured, multidisciplinary plan that outlines a sequence of evidence-based clinical interventions, diagnostic steps, therapeutic options, and/or supportive measures tailored to the specific characteristics of a patient's condition. For oncologic diseases, in some embodiments a care pathway begins with, or is predicated upon, initial diagnostic confirmation through biopsy and imaging studies, identification of actionable mutations through molecular profiling, tumor biomarkers, or resistance patterns. In some embodiments, a care pathway specifies a sequence of treatments, such as surgery, radiation therapy, systemic chemotherapy, targeted therapy (e.g., kinase inhibitors or monoclonal antibodies), or immunotherapy-based on staging, histology, and predictive biomarkers. In some embodiments, a care pathway includes supportive care recommendations such as nutritional support, palliative interventions, pain management, psychosocial counseling, and/or follow-up surveillance protocols. In some embodiments, for subjects with advanced or treatment-refractory cancers, a care pathway may incorporate guidance on clinical trial enrollment, compassionate use programs, or genomic-based matching for investigational therapies.

Beyond cancer, in some embodiments, a care pathway is directed to chronic diseases such as diabetes, heart failure, chronic kidney disease, or autoimmune disorders. For instance, in some embodiments a care pathway for diabetes involves stepwise intensification of glycemic control strategies (e.g., lifestyle modifications, oral hypoglycemics, GLP-1 receptor agonists, insulin therapy), along with screening and management of complications such as retinopathy, nephropathy, and cardiovascular risk. In heart failure, in some embodiments, a care pathway include guideline-directed medical therapy (e.g., beta blockers, ACE inhibitors, SGLT2 inhibitors), monitoring for fluid status and ejection fraction, device therapy (e.g., ICD or CRT), and referrals to cardiology or heart transplant centers as appropriate. Across disease domains, in some embodiments, care pathways incorporate personalized elements such as pharmacogenomics, social determinants of health, and patient preferences to enhance precision, equity, and adherence.

In some embodiments, the care pathways generated by the AI components described herein may not be static but may be dynamically adapted in response to new clinical information, therapeutic responses, or emerging evidence. These care pathways serve as clinically actionable outputs that bridge structured medical data and domain knowledge, supporting physicians in delivering timely, individualized, and evidence-aligned care.

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

As used herein, the terms “cancer state” or “cancer condition” refer to a characteristic of a cancer patient's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). In some embodiments, one or more additional personal characteristics of the subject are used further describe the cancer state or cancer condition of the subject, e.g., age, gender, weight, race, personal habits (e.g., smoking, drinking, diet), other pertinent medical conditions (e.g., high blood pressure, dry skin, other diseases), current medications, allergies, pertinent medical history, current side effects of cancer treatments and other medications, etc.

### Classifier

As used interchangeably herein, the term “classifier” or “model” refers to a machine learning model or algorithm.

In some embodiments, a model includes an unsupervised learning algorithm. One example of an unsupervised learning algorithm is cluster analysis. In some embodiments, a model includes supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm comprises a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xᵢ, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent or backward propagation method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastic et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of ‘kernels’, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎-x₍₀₎∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in McLachlan et al., Bioinformatics 18 (3): 413-422, 2002. In some embodiments, in particular, those embodiments including a temporal component, the model is a hidden Markov model such as described by Schliep et al., 2003, Bioinformatics 19 (1): 1255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s (x, x′) is used to compare two vectors x and x′. In some such embodiments, s (x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥ 4,000; n≥5,000; n≥7,500; n≥10,000; n≥20,000; n≥40,000; n≥75,000; n≥100,000; n≥ 200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the term “classification” can refer to any number(s) or other characters(s) that are associated with a particular property of a sample. For example, in some embodiments, the term “classification” can refer to a type of cancer in a subject, a stage of cancer in a subject, a prognosis for a cancer in a subject, a tumor load, a presence of tumor metastasis in a subject, and the like. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotides within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘mutations.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “gene product” refers to an RNA (e.g., mRNA or miRNA) or protein molecule transcribed or translated from a particular genomic locus, e.g., a particular gene. The genomic locus can be identified using a gene name, a chromosomal location, or any other genetic mapping metric.

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. When referring to mRNA or protein expression, the term generally refers to the amount of any RNA or protein species corresponding to a particular genomic locus, e.g., a particular gene. However, in some embodiments, an expression level can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used herein, the term “ratio” refers to any comparison of a first metric X, or a first mathematical transformation thereof X′ (e.g., measurement of a number of units of a genomic sequence in a first one or more biological samples or a first mathematical transformation thereof) to another metric Y or a second mathematical transformation thereof Y′ (e.g., the number of units of a respective genomic sequence in a second one or more biological samples or a second mathematical transformation thereof) expressed as X/Y, Y/X, log_{N} (X/Y), log_{N} (Y/X), X′/Y, Y/X′, log_{N} (X′/Y), or log_{N} (Y/X′), X/Y′, Y′/X, log_{N} (X/Y′), log_{N} (Y′/X), X′/Y′, Y′/X′, log_{N} (X′/Y′), or log_{N} (Y′/X′), where N is any real number greater than 1 and where example mathematical transformations of X and Y include, but are not limited to. raising X or Y to a power Z, multiplying X or Y by a constant Q, where Z and Q are any real numbers, and/or taking an M based logarithm of X and/or Y, where M is a real number greater than 1. In one non-limiting example, X is transformed to X′ prior to ratio calculation by raising X by the power of two (X²) and Y is transformed to Y′ prior to ratio calculation by raising Y by the power of 3.2 (Y^{3.2}) and the ratio of X and Y is computed as log₂ (X′/Y′).

As used herein, the term “relative abundance” refers to a ratio of a first amount of a compound measured in a sample, e.g., a gene product (an RNA species, e.g., mRNA or miRNA, or protein molecule) or nucleic acid fragments having a particular characteristic (e.g., aligning to a particular locus or encompassing a particular allele), to a second amount of a compound measured in a second sample. In some embodiments, relative abundance refers to a ratio of the amount of a species of a compound to the total amount of the compound in the same sample. For instance, a ratio of the amount of mRNA transcripts encoding a particular gene in a sample (e.g., aligning to a particular region of the exome) to the total amount of mRNA transcripts in the sample. In other embodiments, relative abundance refers to a ratio of an amount of a compound or species of a compound in a first sample to an amount of the compound of the species of the compound in a second sample. For instance, a ratio of a normalized amount of mRNA transcripts encoding a particular gene in a first sample to a normalized amount of mRNA transcripts encoding the particular gene in a second and/or reference sample.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “genetic sequence” refers to a recordation of a series of nucleotides present in a subject's RNA or DNA as determined by sequencing of nucleic acids from the subject.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “bioinformatics pipeline” refers to a series of processing stages used to determine characteristics of a subject's genome or exome based on sequencing data of the subject's genome or exome. A bioinformatics pipeline may be used to determine characteristics of a germline genome or exome of a subject and/or a cancer genome or exome of a subject. In some embodiments, the pipeline extracts information related to genomic alterations in the cancer genome of a subject, which is useful for guiding clinical decisions for precision oncology, from sequencing results of a biological sample, e.g., a tumor sample, liquid biopsy sample, reference normal sample, etc., from the subject. Certain processing stages in a bioinformatics may be ‘connected,’ meaning that the results of a first respective processing stage are informative and/or essential for execution of a second, downstream processing stage. For instance, in some embodiments, a bioinformatics pipeline includes a first respective processing stage for identifying genomic alterations that are unique to the cancer genome of a subject and a second respective processing stage that uses the quantity and/or identity of the identified genomic alterations to determine a metric that is informative for precision oncology, e.g., a tumor mutational burden. In some embodiments, the bioinformatics pipeline includes a reporting stage that generates a report of relevant and/or actionable information identified by upstream stages of the pipeline, which may or may not further include recommendations for aiding clinical therapy decisions.

As used herein, an “actionable genomic alteration” or “actionable variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to be associated with a therapeutic course of action that is more likely to produce a positive effect in a cancer patient that has the actionable variant than in a similarly situated cancer patient that does not have the actionable variant. For instance, administration of EGFR inhibitors (e.g., afatinib, erlotinib, gefitinib) is more effective for treating non-small cell lung cancer in patients with an EGFR mutation in exons 19/21 than for treating non-small cell lung cancer in patients that do not have an EGFR mutations in exons 19/21. Accordingly, an EGFR mutation in exon 19/21 is an actionable variant. In some instances, an actionable variant is only associated with an improved treatment outcome in one or a group of specific cancer types. In other instances, an actionable variant is associated with an improved treatment outcome in substantially all cancer types.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

Reference will now be made in detail to embodiments, examples of which are illustrated in the accompanying drawings. In the following detailed description, numerous specific details are set forth in order to provide a thorough understanding of the present disclosure, including example systems, methods, techniques, instruction sequences, and computing machine program products that embody illustrative implementations. However, the illustrative discussions below are not intended to be exhaustive or to limit the implementations to the precise forms disclosed. Many modifications and variations are possible in view of the above teachings. The features described herein are not limited by the illustrated ordering of acts or events, as some acts can occur in different orders and/or concurrently with other acts or events.

The implementations provided herein are chosen and described in order to best explain the principles and their practical applications, to thereby enable others skilled in the art to best utilize the various embodiments with various modifications as are suited to the particular use contemplated. In some instances, well-known methods, procedures, components, circuits, and networks have not been described in detail so as not to unnecessarily obscure aspects of the embodiments. In other instances, it will be apparent to one of ordinary skill in the art that the present disclosure may be practiced without one or more of the specific details.

It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that though such a design effort might be complex and time-consuming, it will nevertheless be a routine undertaking of engineering for those of ordering skill in the art having the benefit of the present disclosure.

### Example System Embodiments

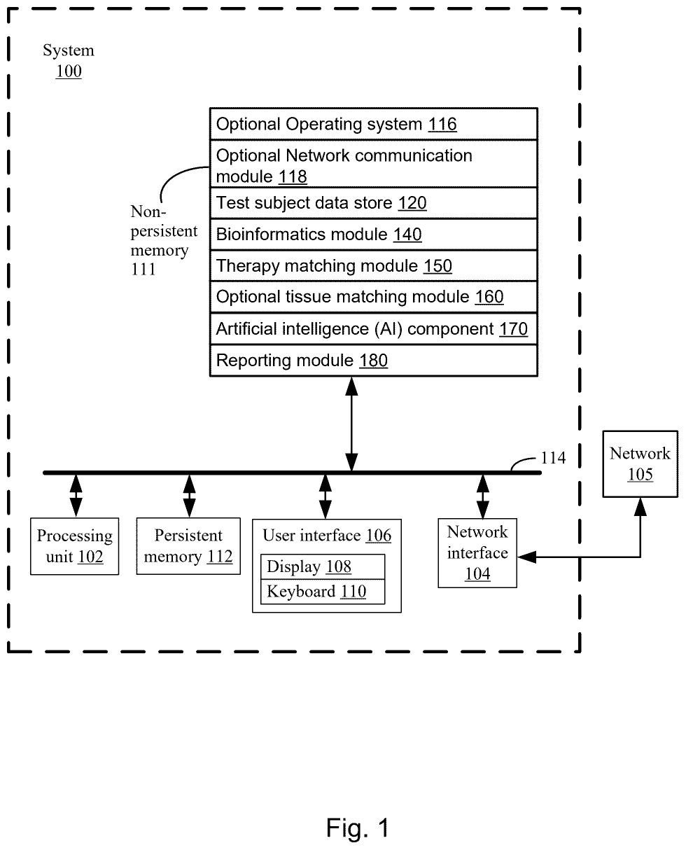

Now that an overview of some aspects of the present disclosure and some definitions used in the present disclosure have been provided, details of an exemplary system for providing clinical support for personalized therapy by combining electronic medical record data and tissue modeling data are now described in conjunction with FIGS. 1A-1D. FIGS. 1A-ID collectively illustrate the topology of an example system for providing clinical support for personalized therapy by combining electronic medical record data and tissue modeling data, in accordance with some embodiments of the present disclosure. Advantageously, the example system illustrated in FIGS. 1A-ID improves upon conventional methods for providing clinical support for personalized therapy by better accounting for the patient's biology.

FIG. 1A is a block diagram illustrating a system in accordance with some implementations. The device 100 in some implementations includes one or more processing units CPU(s) 102 (also referred to as processors), one or more network interfaces 104, a user interface 106, e.g., including a display 108 and/or an input 110 (e.g., a mouse, touchpad, keyboard, etc.), a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 105;
a test subject data store 120 for storing one or more collections of features from patients (e.g., subjects);
a bioinformatics module 140 for processing sequencing data and extracting features from sequencing data, e.g., from biopsy sequencing assays;
a therapy matching module 150 for matching possible therapeutics based on a patient's biology;
an optional tissue matching module 160 for evaluating patient features, e.g., genomic alterations, compound genomic features, and clinical features;
an artificial intelligence (AI) component 170 for predicting care pathway options; and
a reporting module 180 for generating and transmitting reports that provide clinical support for personalized cancer therapy.

Although FIGS. 1A-1D depict a “system 100,” the figures are intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112. For example, in various implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of system 100, that is addressable by system 100 so that system 100 may retrieve all or a portion of such data when needed.

For purposes of illustration in FIG. 1A, system 100 is represented as a single computer that includes all of the functionality for providing clinical support for personalized cancer therapy. However, while a single machine is illustrated, the term “system” shall also be taken to include any collection of machines that individually or jointly execute a set (or multiple sets) of instructions to perform any one or more of the methodologies discussed herein.

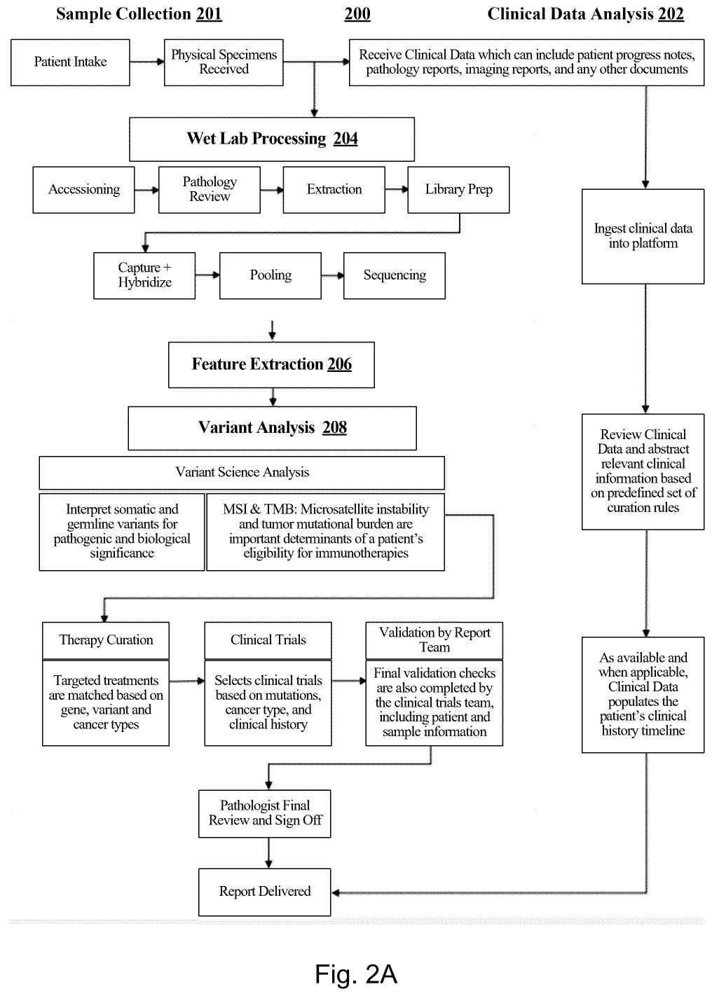

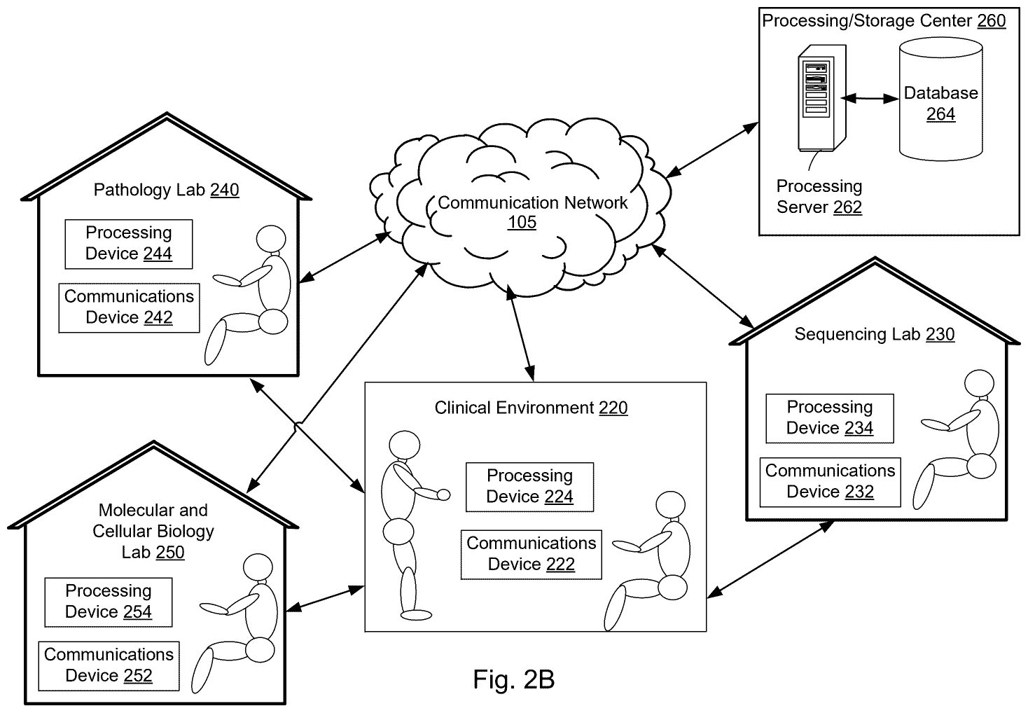

For example, in some embodiments, system 100 includes one or more computers. In some embodiments, the functionality for providing clinical support for personalized cancer therapy is spread across any number of networked computers and/or resides on each of several networked computers and/or is hosted on one or more virtual machines at a remote location accessible across the communications network 105. For example, different portions of the various modules and data stores illustrated in FIGS. 1A-1D can be stored and/or executed on the various instances of a processing device and/or processing server/database in the distributed diagnostic environment 210 illustrated in FIG. 2B (e.g., processing devices 224, 234, 244, and 254, processing server 262, and database 264).

The system may operate in the capacity of a server or a client machine in client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

One of skill in the art will appreciate that any of a wide array of different computer topologies are used for the application and all such topologies are within the scope of the present disclosure.

Further details on systems and exemplary embodiments of modules and feature collections are discussed in International Patent Application No. WO 2020/142551 entitled “A METHOD AND PROCESS FOR PREDICTING AND ANALYZING PATIENT COHORT RESPONSE, PROGRESSION, AND SURVIVAL,” published Jul. 9, 2020, which is hereby incorporated herein by reference in its entirety.

### Example Methods

Now that details of a system 100 for providing clinical support for personalized therapy, e.g., by combining tissue modeling data with information electronic medical records, have been disclosed, details regarding processes and features of the system, in accordance with various embodiments of the present disclosure, are disclosed below. Specifically, example processes are described below with reference to FIGS. 2-8. In some embodiments, such processes and features of the system are carried out by modules as illustrated in FIG. 1. Referring to these methods, the systems described herein (e.g., system 100) include instructions for combining tissue modeling data with information electronic medical records.

### FIG. 2B: Distributed Diagnostic and Clinical Environment

In some aspects, the methods described herein for providing clinical support for personalized therapy are performed across a distributed diagnostic/clinical environment, e.g., as illustrated in FIG. 2B. However, in some embodiments, the improved methods described herein, which combine tissue modeling data with electronic medical records for identifying care pathways, are performed at a single location, e.g., at a single computing system or environment, although ancillary procedures supporting the methods described herein, and/or procedures that make further use of the results of the methods described herein, may be performed across a distributed diagnostic/clinical environment.

FIG. 2B illustrates an example of a distributed diagnostic/clinical environment 210. In some embodiments, the distributed diagnostic/clinical environment is connected via communication network 105. In some embodiments, one or more biological samples, e.g., one or more liquid biopsy samples, solid tumor biopsy, normal tissue samples, and/or control samples, are collected from a subject in clinical environment 220, e.g., a doctor's office, hospital, or medical clinic, or at a home health care environment (not depicted). In some embodiments, one or more biological samples, or portions thereof, are processed within the clinical environment 220 where collection occurred, using a processing device 224, e.g., a nucleic acid sequencer for obtaining sequencing data, a microscope for obtaining pathology data, a mass spectrometer for obtaining proteomic data, etc. The information obtained through these analyses is often aggregated in electronic medical records for the patient.

In some embodiments, one or more biological samples, or portions thereof are sent to one or more external environments, e.g., sequencing lab 230, pathology lab 240, and/or molecular biology lab 250, each of which includes a processing device 234, 244, and 254, respectively, to generate biological data 121 for the subject. Each environment includes a communications device 222, 232, 242, and 252, respectively, for communicating biological data 121 about the subject to a processing server 262 and/or database 264, which may be located in yet another environment, e.g., processing/storage center 260. Human tissue modeling, e.g., using cell lines, organoids, tissue slice cultures, PDX models, microfluidics, etc. can be performed at one or more external environment, e.g., molecular biology lab 250. Thus, in some embodiments, different portions of the systems and methods described herein are fulfilled by different processing devices located in different physical environments.

Accordingly, in some embodiments, a method for providing clinical support for personalized cancer therapy, e.g., by combining human tissue modeling data with electronic health records, is performed across one or more environments, as illustrated in FIG. 2B. For instance, in some such embodiments, a liquid biopsy sample is collected at clinical environment 220 or in a home healthcare environment. The sample, or a portion thereof, is sent to sequencing lab 230 where raw sequence reads of nucleic acids in the sample are generated by sequencer 234. The raw sequencing data is communicated, e.g., from communications device 232, to database 264 at processing/storage center 260, where processing server 262 extracts features from the sequence reads by executing one or more of the processes in a bioinformatics module, thereby generating genomic features for the sample that are stored in an electronic medical record. A healthcare provider may access such an electronic medical record and/or tissue modeling data at a processing server 262, where models, e.g., generative AI systems, combine patient information from the medical records with human tissue modeling data to identify care pathways for treating or managing a disorder in patient. The medical professional may access the electronic medical record and tissue modeling data at processing/storage center 260 or through communications network 105. After identification of a care pathway, a clinical report including the identified care pathway can be transmitted to a medical professional, e.g., an oncologist, at clinical environment 220, who uses the report to support clinical decision making for personalized treatment of the patient's cancer.

In some embodiments, the medical professional accesses a model stored at processing/storage center 260 to combine tissue modeling data with an electronic medical record directly from clinical environment 220. Examples of systems that facilitate remote access for matching therapies to patients are described in U.S. Pat. Nos. 11,100,933 and 11,705,226, as well as U.S. Patent Application Publication No. US 2022/0059240, the disclosure of which are incorporated herein by reference in their entireties.

### FIG. 2A: Example Workflow for Precision Medicine

FIG. 2A is a flowchart of an example workflow 200 for collecting and analyzing data in order to generate a clinical report 139 to support clinical decision making in precision medicine. Briefly, the workflow begins with patient intake and sample collection 201, where one or more liquid biopsy samples, one or more tumor biopsy, and one or more normal and/or control tissue samples are collected from the patient (e.g., at a clinical environment 220 or home healthcare environment, as illustrated in FIG. 2B). In some embodiments, personal data corresponding to the patient and a record of the one or more biological samples obtained (e.g., patient identifiers, patient clinical data, sample type, sample identifiers, cancer conditions, etc.) are entered into a data analysis platform, e.g., test subject data store 120. Accordingly, in some embodiments, the methods disclosed herein include obtaining one or more biological samples from one or more subjects, e.g., cancer patients. In some embodiments, the subject is a human, e.g., a human cancer patient.

In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, blood samples are collected from patients in commercial blood collection containers. In some embodiments, saliva samples are collected from patients in commercial saliva collection containers.

In some embodiments, one or more biological samples collected from the patient is a solid tissue sample, e.g., a solid tumor sample or a solid normal tissue sample. Methods for obtaining solid tissue samples, e.g., of cancerous and/or normal tissue are known in the art and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subject's mouth and rubbing it against their check, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

The biological samples collected from the patient are, optionally, sent to various analytical environments (e.g., sequencing lab 230, pathology lab 240, and/or molecular biology lab 250) for processing (e.g., data collection) and/or analysis (e.g., feature extraction). Wet lab processing 204 may include cataloguing samples (e.g., accessioning), examining clinical features of one or more samples (e.g., pathology review), and nucleic acid sequence analysis (e.g., extraction, library prep, capture+hybridize, pooling, and sequencing). In some embodiments, the workflow includes clinical analysis of one or more biological samples collected from the subject, e.g., at a pathology lab 240 and/or a molecular and cellular biology lab 250, to generate clinical features such as pathology features, imaging data, and/or tissue culture/organoid data.

In some embodiments, the pathology data collected during clinical evaluation includes visual features identified by a pathologist's inspection of a specimen (e.g., a solid tumor biopsy), e.g., of stained H&E or IHC slides. In some embodiments, the sample is a solid tissue biopsy sample. In some embodiments, the tissue biopsy sample is a formalin-fixed tissue (FFT), e.g., a formalin-fixed paraffin-embedded (FFPE) tissue. In some embodiments, the tissue biopsy sample is an FFPE or FFT block. In some embodiments, the tissue biopsy sample is a fresh-frozen tissue biopsy. The tissue biopsy sample can be prepared in thin sections (e.g., by cutting and/or affixing to a slide), to facilitate pathology review (e.g., by staining with immunohistochemistry stain for IHC review and/or with hematoxylin and cosin stain for H&E pathology review). For instance, analysis of slides for H&E staining or IHC staining may reveal features such as tumor infiltration, programmed death-ligand 1 (PD-L1) status, human leukocyte antigen (HLA) status, or other immunological features.

Further details on methods, systems, and algorithms for using pathology data to classify cancer and identify targeted therapies are discussed, for example, in U.S. Pat. Nos. 10,957,041; 11,244,763; 11,848,107; and 11,145,416, the contents of which are hereby incorporated by reference, in their entireties, for all purposes.

In some embodiments, imaging data collected during clinical evaluation includes features identified by review of in-vitro and/or in-vivo imaging results (e.g., of a tumor site), for example a size of a tumor, tumor size differentials over time (such as during treatment or during other periods of change). In some embodiments, imaging data includes features determined using machine learning algorithms to evaluate imaging data collected as described above.

Further details on methods, systems, and algorithms for using medical imaging to classify cancer and identify targeted therapies are discussed, for example, in are discussed, for example, in U.S. Pat. Nos. 10,957,041, 11,244,763; 11,848,107; and 11,145,416, the contents of which are hereby incorporated by reference, in their entireties, for all purposes.

In some embodiments, tissue culture/organoid data is collected from tissue samples (e.g., tumor samples) collected during a clinical evaluation. For instance, in some embodiments, tissue samples obtained from the patients (e.g., tumor tissue, normal tissue, or both) are cultured (e.g., in liquid culture, solid-phase culture, and/or organoid culture) and various features, such as cell morphology, growth characteristics, genomic alterations, and/or drug sensitivity, are evaluated. In some embodiments, tissue culture/organoid data includes features determined using machine learning algorithms to evaluate tissue culture/organoid data collected as described above. Examples of tissue organoid (e.g., personal tumor organoid) culturing and feature extractions thereof are described in U.S. patent application Ser. No. 17/771,401, filed on Oct. 22, 2020, and U.S. Pat. No. 11,629,385, the contents of which are hereby incorporated by reference, in their entireties, for all purposes.

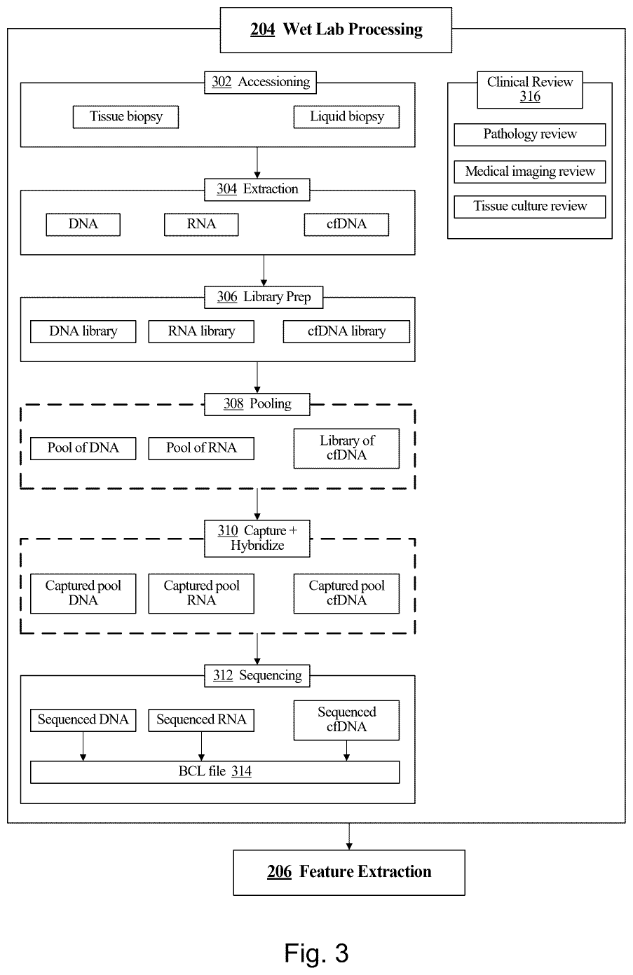

Nucleic acid sequencing of one or more samples collected from the subject is performed, e.g., at sequencing lab 230, during wet lab processing 204. An example workflow for nucleic acid sequencing is illustrated in FIG. 3. In some embodiments, the one or more biological samples obtained at the sequencing lab 230 are accessioned (302), to track the sample and data through the sequencing process.

Next, nucleic acids, e.g., RNA and/or DNA are extracted (304) from the one or more biological samples. Methods for isolating nucleic acids from biological samples are known in the art, and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. For example, acid guanidinium thiocyanate-phenol-chloroform extraction (see, for example, Chomczynski and Sacchi, 2006, Nat Protoc, 1 (2): 581-85, which is hereby incorporated by reference herein), and silica bead/glass fiber adsorption (see, for example, Poeckh et al., 2008, Anal Biochem., 373 (2): 253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed.

In some embodiments where the biological sample is a liquid biopsy sample, e.g., a blood or blood plasma sample, cfDNA is isolated from blood samples using commercially available reagents, including proteinase K, to generate a liquid solution of cfDNA.

In some embodiments, isolated DNA molecules are mechanically sheared to an average length using an ultrasonicator (for example, a Covaris ultrasonicator). In some embodiments, isolated nucleic acid molecules are analyzed to determine their fragment size, e.g., through gel electrophoresis techniques and/or the use of a device such as a LabChip GX Touch. The skilled artisan will know of an appropriate range of fragment sizes, based on the sequencing technique being employed, as different sequencing techniques have differing fragment size requirements for robust sequencing. In some embodiments, quality control testing is performed on the extracted nucleic acids (e.g., DNA and/or RNA), e.g., to assess the nucleic acid concentration and/or fragment size. For example, sizing of DNA fragments provides valuable information used for downstream processing, such as determining whether DNA fragments require additional shearing prior to sequencing.

Wet lab processing 204 then includes preparing a nucleic acid library from the isolated nucleic acids (e.g., cfDNA, DNA, and/or RNA). For example, in some embodiments, DNA libraries (e.g., gDNA and/or cfDNA libraries) are prepared from isolated DNA from the one or more biological samples. In some embodiments, the DNA libraries are prepared using a commercial library preparation kit.

In some embodiments, during library preparation, adapters (e.g., UDI adapters or UMI adapters such as full length or stubby Y adapters) are ligated onto the nucleic acid molecules. In some embodiments, the adapters include unique molecular identifiers (UMIs), which are short nucleic acid sequences (e.g., 3-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, e.g., when multiplex sequencing will be used to sequence DNA from a plurality of samples (e.g., from the same or different subjects) in a single sequencing reaction, a patient-specific index is also added to the nucleic acid molecules. In some embodiments, the patient specific index is a short nucleic acid sequence (e.g., 3-20 nucleotides) that are added to ends of DNA fragments during library construction, that serve as a unique tag that can be used to identify sequence reads originating from a specific patient sample.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries are amplified and purified using commercial reagents. In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device and the resulting flow cell containing amplified target-captured DNA libraries is sequenced on a next generation sequencer to a unique on-target depth selected by the user. In some embodiments, DNA library preparation is performed with an automated system, using a liquid handling robot.

In some embodiments, where feature data 125 includes methylation states 132 for one or more genomic locations, nucleic acids isolated from the biological sample (e.g., cfDNA) are treated to convert unmethylated cytosines to uracils, e.g., prior to generating the sequencing library. Accordingly, when the nucleic acids are sequenced, all cytosines called in the sequencing reaction were necessarily methylated, since the unmethylated cytosines were converted to uracils and accordingly would have been called as thymidines, rather than cytosines, in the sequencing reaction. Commercial kits are available for bisulfite-mediated conversion of methylated cytosines to uracils. Commercial kits are also available for enzymatic conversion of methylated cytosines to uracils.

In some embodiments, wet lab processing 204 includes pooling (308) DNA molecules from a plurality of libraries, corresponding to different samples from the same and/or different patients, to forming a sequencing pool of DNA libraries. When the pool of DNA libraries is sequenced, the resulting sequence reads correspond to nucleic acids isolated from multiple samples. The sequence reads can be separated into different sequence read files, corresponding to the various samples represented in the sequencing read based on the unique identifiers present in the added nucleic acid fragments. In this fashion, a single sequencing reaction can generate sequence reads from multiple samples. Advantageously, this allows for the processing of more samples per sequencing reaction.

In some embodiments, wet lab processing 204 includes enriching (310) a sequencing library, or pool of sequencing libraries, for target nucleic acids, e.g., nucleic acids encompassing loci that are informative for precision oncology and/or used as internal controls for the sequencing or bioinformatics processes. In some embodiments, enrichment is achieved by hybridizing target nucleic acids in the sequencing library to probes that hybridize to the target sequences, and then isolating the captured nucleic acids away from off-target nucleic acids that are not bound by the capture probes. Of course, some off-target nucleic acids will remain in the final sequencing pool.

Advantageously, enriching for target sequences prior to sequencing nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample.

In some embodiments, the enrichment is performed prior to pooling multiple nucleic acid sequencing libraries. However, in other embodiments, the enrichment is performed after pooling nucleic acid sequencing libraries, which has the advantage of reducing the number of enrichment assays that have to be performed.

In some embodiments, the enrichment is performed prior to generating a nucleic acid sequencing library. This has the advantage that fewer reagents are needed to perform both the enrichment (because there are fewer target sequences at this point, prior to library amplification) and the library production (because there are fewer nucleic acid molecules to tag and amplify after the enrichment). However, this raises the possibility of pull-down bias and/or that small variations in the enrichment protocol will result in less consistent results.

Generally, probes for enrichment of nucleic acids (e.g., cfDNA obtained from a liquid biopsy sample) include DNA, RNA, or a modified nucleic acid structure with a base sequence that is complementary to a locus of interest. For instance, a probe designed to hybridize to a locus in a cfDNA molecule can contain a sequence that is complementary to either strand, because the cfDNA molecules are double stranded. In some embodiments, each probe in the plurality of probes includes a nucleic acid sequence that is identical or complementary to at least 10, at least 11, at least 12, at least 13, at least 14, or at least 15 consecutive bases of a locus of interest. In some embodiments, each probe in the plurality of probes includes a nucleic acid sequence that is identical or complementary to at least 20, 25, 30, 40, 50, 75, 100, 150, 200, or more consecutive bases of a locus of interest.

Sequence reads are then generated (312) from the sequencing library or pool of sequencing libraries. Sequencing data may be acquired by any methodology known in the art. For example, next generation sequencing (NGS) techniques such as sequencing-by-synthesis technology, pyrosequencing (454), ion semiconductor technology, single-molecule real-time sequencing, sequencing by ligation, nanopore sequencing, or paired-end sequencing. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Next-generation sequencing produces millions of short reads (e.g., sequence reads) for each biological sample. Accordingly, in some embodiments, the plurality of sequence reads obtained by next-generation sequencing of cfDNA molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides. In some embodiments, the sequence reads have an average length of between 50 nucleotides and 500 nucleotides. In some embodiments, the sequence reads have an average length of between 75 nucleotides and 475 nucleotides. In some embodiments, the sequence reads have an average length of between 100 nucleotides and 450 nucleotides. In some embodiments, the sequence reads have an average length of between 150 nucleotides and 425 nucleotides. In some embodiments, the sequence reads have an average length of between 200 nucleotides and 400 nucleotides. In some embodiments, the sequence reads have an average length of at least 50, 60, 70, 80, 90, 100, 150, 200, 250, 300, or more nucleotides.

In some embodiments, sequencing is performed after enriching for nucleic acids (e.g., cfDNA, gDNA, and/or RNA) encompassing a plurality of predetermined target sequences, e.g., human genes and/or non-coding sequences associated with cancer. Advantageously, sequencing a nucleic acid sample that has been enriched for target nucleic acids, rather than all nucleic acids isolated from a biological sample, significantly reduces the average time and cost of the sequencing reaction. Accordingly, in some preferred embodiments, the methods described herein include obtaining a plurality of sequence reads of nucleic acids that have been hybridized to a probe set for hybrid-capture enrichment (e.g., of one or more genes listed in Table 1).

In some embodiments, panel-targeting sequencing is performed to an average on-target depth of at least 500×, at least 750×, at least 1000×, at least 2500×, at least 500×, at least 10,000×, or greater depth. In some embodiments, samples are further assessed for uniformity above a sequencing depth threshold (e.g., 95% of all targeted base pairs at 300x sequencing depth). In some embodiments, the sequencing depth threshold is a minimum depth selected by a user or practitioner.

In some embodiments, the sequence reads are obtained by a whole genome or whole exome sequencing methodology. In some such embodiments, whole exome capture is performed with an automated system, using a liquid handling robot. Whole genome sequencing, and to some extent whole exome sequencing, is typically performed at lower sequencing depth than smaller target-panel sequencing reactions, because many more loci are being sequenced. For example, in some embodiments, whole genome or whole exome sequencing is performed to an average sequencing depth of at least 3×, at least 5×, at least 10×, at least 15×, at least 20×, or greater. In some embodiments, low-pass whole genome sequencing (LPWGS) techniques are used for whole genome or whole exome sequencing. LPWGS is typically performed to an average sequencing depth of about 0.25× to about 5×, more typically to an average sequencing depth of about 0.5× to about 3×.

Because of the differences in the sequencing methodologies, data obtained from targeted-panel sequencing is better suited for certain analyses than data obtained from whole genome/whole exome sequencing, and vice versa. For instance, because of the higher sequencing depth achieved by targeted-panel sequencing, the resulting sequence data is better suited for the identification of variant alleles present at low allelic fractions in the sample, e.g., less than 20%. By contrast, data generated from whole genome/whole exome sequencing can be better suited for the estimation of genome-wide metrics with higher accuracy, such as tumor mutational burden, because the entire genome is better represented in the sequencing data. Accordingly, in some embodiments, a nucleic acid sample, e.g., a cfDNA, gDNA, or mRNA sample, is evaluated using both targeted-panel sequencing and whole genome/whole exome sequencing (e.g., LPWGS).

While workflow 200 illustrates obtaining a biological sample, extracting nucleic acids from the biological sample, and sequencing the isolated nucleic acids, in some embodiments, sequencing data used in the improved systems and methods described herein (e.g., which include improved methods for determining accurate circulating tumor fraction estimates) is obtained by receiving previously generated sequence reads, in electronic form.

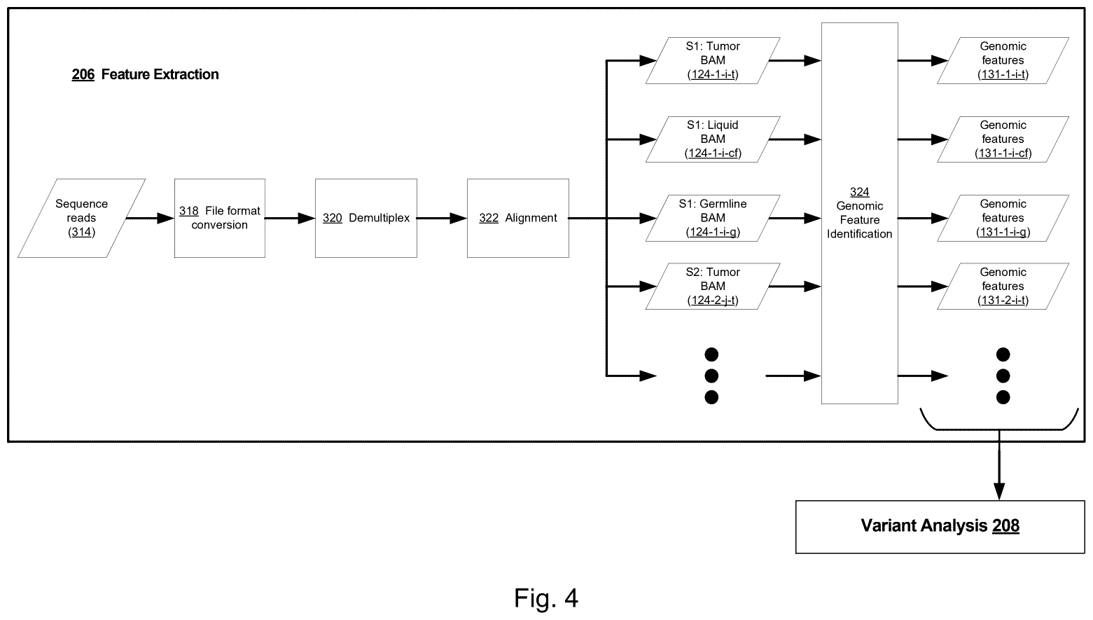

Referring again to FIG. 2A, nucleic acid sequencing data 122 generated from the one or more patient samples is then evaluated (e.g., via variant analysis 206) in a bioinformatics pipeline, e.g., using bioinformatics module 140 of system 100, to identify genomic alterations and other metrics in the cancer genome of the patient. An example overview for a bioinformatics pipeline is described below with respect to FIG. 4.

FIG. 4 illustrates an example bioinformatics pipeline 206 (e.g., as used for feature extraction in the workflows illustrated in FIGS. 2A and 3) for providing clinical support for precision medicine. As shown in FIG. 4, sequencing data 122 obtained from the wet lab processing 204 (e.g., sequence reads 314) is input into the pipeline.

In various embodiments, the bioinformatics pipeline includes a circulating tumor DNA (ctDNA) pipeline for analyzing liquid biopsy samples. The pipeline may detect SNVs, INDELs, copy number amplifications/deletions and genomic rearrangements (for example, fusions). The pipeline may employ unique molecular index (UMI)-based consensus base calling as a method of error suppression as well as a Bayesian tri-nucleotide context-based position level error suppression. In various embodiments, it is able to detect variants having a 0.1%, 0.15%, 0.2%, 0.25%, 0.3%, 0.4%, or 0.5% variant allele fraction.

### Variant Identification

In some embodiments, variant analysis of aligned sequence reads, e.g., in SAM or BAM format, includes identification of single nucleotide variants (SNVs), multiple nucleotide variants (MNVs), indels (e.g., nucleotide additions and deletions), and/or genomic rearrangements (e.g., inversions, translocations, and gene fusions) using a variant identification module, e.g., which includes a SNV/MNV calling algorithm, an indel calling algorithm, and/or one or more genomic rearrangement calling algorithms. Essentially, the module first identifies a difference between the sequence of an aligned sequence read and the reference sequence to which the sequence read is aligned (e.g., an SNV/MNV, an indel, or a genomic rearrangement) and makes a record of the variant, e.g., in a variant call format (VCF) file. For instance, software packages are used to call variants using sorted BAM files and reference BED files as the input. In some embodiments, a raw VCF file (variant call format) file is output, showing the locations where the nucleotide base in the sample is not the same as the nucleotide base in that position in the reference sequence construct.

### Concurrent Testing

Unless stated otherwise, as used herein, the term “concurrent” as it relates to assays refers to a period of time between zero and ninety days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 90 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 60 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 30 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 21 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 14 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 7 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 3 days.

### Example Methods for Querying Electronic Medical Records.

Considering the extensive volume of text contained within a real-world data (RWD) warehouse of EHRs, in some embodiments a retrieval-augmented generative (RAG) approach is used to identify relevant portions of EHR text, e.g., relevant portions of unstructured clinical notes. A RAG approach proves to be more efficient and effective than providing the model with larger context windows. In some embodiments, RAG is a two-step process that involves retrieving relevant documents from a corpus (e.g., a large corpus with thousands or millions of documents) and then feeding the retrieved documents into a model to generate an analysis and response.

In some embodiments, clinical notes from an EHR are divided into individual segments, also referred to herein as snippets. One example method for segmenting unstructured clinical data (e.g., clinical notes) includes tokenizing the unstructured clinical data to obtain a plurality of tokens and segmenting the plurality of tokens to obtain a plurality of segments (snippets), e.g., where each respective segment in the plurality of segments has approximately a same number of tokens.

In some embodiments, the individual snippets are evaluated to determine whether they include information pertinent to determining whether the subject has a target medical condition. In some embodiments, the evaluation is performed by natural language processing. In some embodiments, the evaluation is performed based on pattern recognition of regular expressions (Regex) related to the target medical condition. In some embodiments, the use of Regex avoids introducing bias through additional hyperparameter tuning and narrows the focus to assessing the model's capability in diagnosing diseases. However, other retrieval models can be used instead of, or in addition to, Regex. For example, in some embodiments, the snippets are evaluated using Term Frequency-Inverse Document Frequency. In some embodiments, the snippets are evaluated using Cohere's re-rank. In some embodiments, the snippets are evaluated using Instructor embeddings.

In some embodiments, the snippets are retrieved by using a model (e.g., an LLM) to identify portions of a medical record that include information relating to the target medical condition. In some embodiments, a prompt is given to the model to identify any portion of a medical record that is relevant to an indication of the disease diagnosis. In some embodiments, the identified portion (e.g., snippet) is defined to be within a specific range of characters. For example, in some embodiments, the identified portion must be from X to Y characters in length, where X is a minimum length and Y is a maximum length. In some embodiments, the identified portion (e.g., snippet) is defined to be within a specific range of token length. For example, in some embodiments, the identified portion must be from X to Y tokens in length, where X is a minimum length and Y is a maximum length. In some embodiments, the identified portion (e.g., snippet) must satisfy a relevance threshold. For instance, in some embodiments, a set of candidate portions are identified and ranked in terms of relevance to the medical condition relative to each other and the top X number of candidate portions are selected for retrieval. In some embodiments, the ranking is limited to portions obtained from a single document within a medical record. In some embodiments, the ranking is applied across a plurality of documents within the medical record.

While the RAG approach reduces the amount of text processed by the model, RWD clinical notes often comprise many pages of text. Consequently, the Regex retriever is still likely to return a large number of snippets determined to include information pertinent to determining whether the subject has a target medical condition, which may exceed the model's context window. In some embodiments, a map-reduce approach is employed to address this issue. Map-reduce allows for parallel execution of the model on individual snippets, improving efficiency and reducing processing time. It also facilitates handling of large numbers of identified snippets by distributing the processing load across multiple iterations. By generating individual outputs for each snippet, the chain can extract specific information that contributes to a more comprehensive final result.

Accordingly, in some embodiments, each identified snippet is presented as context to the model, along with a set of instructions to facilitate decision-making. For example, in some embodiments, the model is prompted to determine whether the snippet indicates that the subject has a specific medical condition, such as a specific cancer type, pulmonary hypertension (PH), or another medical condition. The prompt may instruct the model to respond in a binary format (e.g., yes or no), or in a ternary format (yes, no, or uncertain). In some embodiments, the prompt further instructs the model to support its answer with evidence (e.g., from the snippet) effectively summarizing the relevant portion and reducing the amount of context passed to a subsequent model (e.g., in a map-reduce model chain). In other embodiments, by the prompt may go beyond diagnosis and ask the model to identify the most appropriate next therapy or care pathway for the patient, based on clinical, molecular, organoid/in vitro or in silico modeling data, and optionally informed by clinical guidelines or trial response data from similar patients. For example, the model may be presented with a list of two or more candidate therapies or care strategies and asked to select the one most likely to elicit a favorable patient response, along with supporting evidence drawn from the same contextual data.

In some embodiments, the prompt includes a statement that steers the model. For example, referring again to the example of phenotyping for pulmonary hypertension, in some embodiments, the prompt instructs the model to count a ‘possible’ case of PH as ‘no’ answer. In some embodiments, the prompt instructs the model to count a clinical note of a history of PH as a ‘yes’ answer. In some embodiments, the model is further provided with examples of evidence that indicate the presence of the target medical condition. In some embodiments, the model is further provided with examples of evidence that do not indicate the presence of the target medical condition. In some embodiments, the model is further provided with evidence that indicates the absence of the target medical condition. In some embodiments, the prompt includes a Chain-of-Thought (CoT) phrase. Use of CoT enhances reasoning by models in some embodiments.

Outputs generated for individual snippets by the model are then aggregated to formulate the final decision. In some embodiments, the aggregation is performed using a model. In some embodiments, the model is provided the outputs from the snippet evaluation as context and is provided the same instructional prompts as for the evaluation of the individual snippets. In some embodiments, the model is provided the outputs from the snippet evaluation as context but is provided different instructional prompts as for the evaluation of the individual snippets. For example, in some embodiments, the model is asked whether any of the outputs from the snippet evaluation indicate a positive diagnosis for the target medical condition. In some embodiments, the aggregation is a max aggregation function, which checks if any of the individual snippet queries returned a positive diagnosis and, if so, assigns a positive label to the patient as whole.

In some embodiments, the snippet evaluation and aggregation step are performed using the same model. In some embodiments, the snippet evaluation and aggregation steps are performed by the model after a single prompt asking the model whether the subject has the medical condition based on evidence contained within the snippets. In some embodiments, the snippet evaluation and aggregation step are performed in series, such that the model is provided separate prompts for the two steps. In some embodiments, the snippet evaluation and aggregation step are performed using different models.

In some embodiments, a user prompt is received at an API with instructions to retrieve snippets and then present them to an AI component responsive to a user prompt. In some embodiments, the API receives a prompt relating to a first subject or group of subjects. In some embodiments, medical records for the subject or group of subjects have already been parsed (snippetized) and snippets saved to a curated database. In some embodiments, the snippitized records have also been sorted to identify snippets related to a target medical condition, e.g., in the curated database. In some such cases, the API retrieves the presorted snippets from the database and presents them to an AI component. In other embodiments, where the medical records have not been snippetized, the API retrieves the medical record and directs a module (e.g., a natural language processing module) to parse the medical record into snippets and optionally sort the snippets to identify those snippets related to the target medical condition. Similarly, in some embodiments where the medical records have been snippetized but have not been sorted, the API retrieves the snippets and directs a module (e.g., a natural language processing module) to identify those snippets related to the target medical condition. The API then presents the identified snippets to the AI component (e.g., a model such as an LLM) in parallel (e.g., via separate instances of the AI component) or sequentially and asks the AI component whether each snippet indicates that the subject has the target medical condition, and optionally to provide reasoning for the answer. The AI component generates answers for each of the snippets and optionally the secondary logic (reasoning) for each answer. The API also includes instructions for aggregating the component answers into a final answer as to whether the subject has the target medical condition. In some embodiments, the API asks the model to aggregate the component answers, and optional secondary logic, such that the AI component may not provide component answers externally, but rather returns a single answer for the subject, which is returned as the response to the API prompt containing the query.

### Systems and Methods for Identifying Care Pathways for Treating a Disorder in a Subject

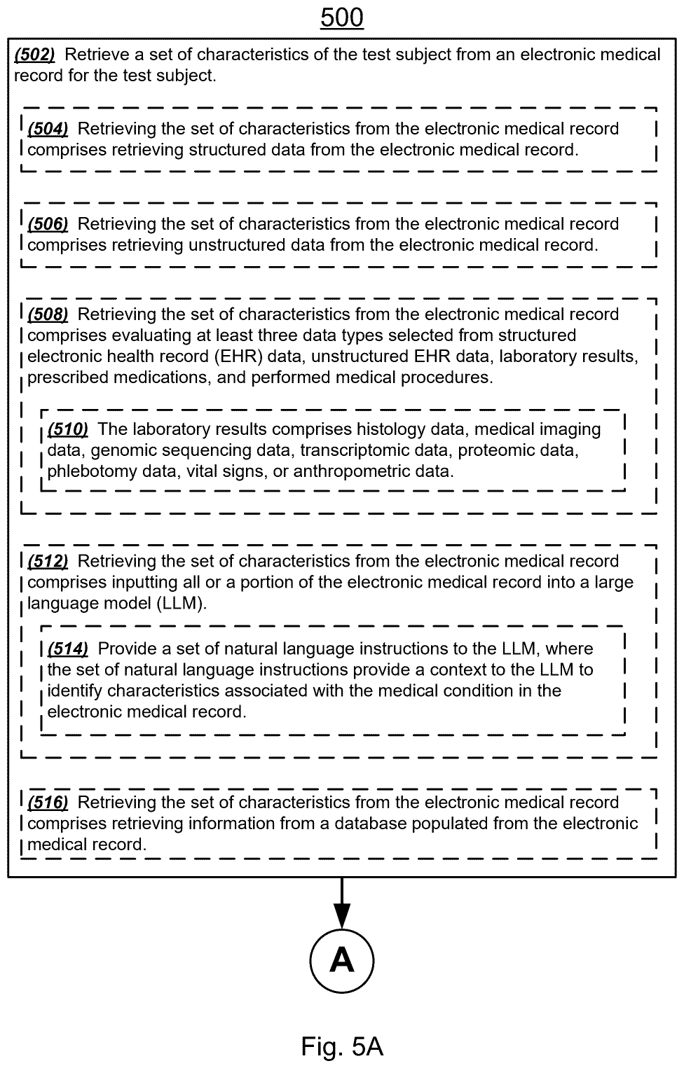

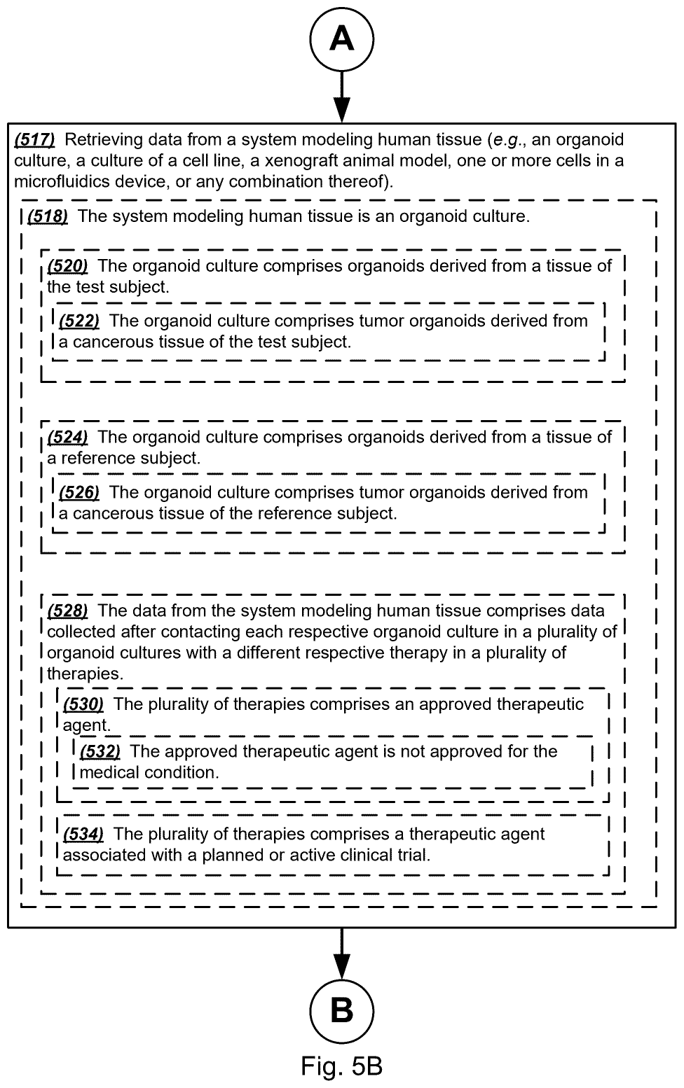

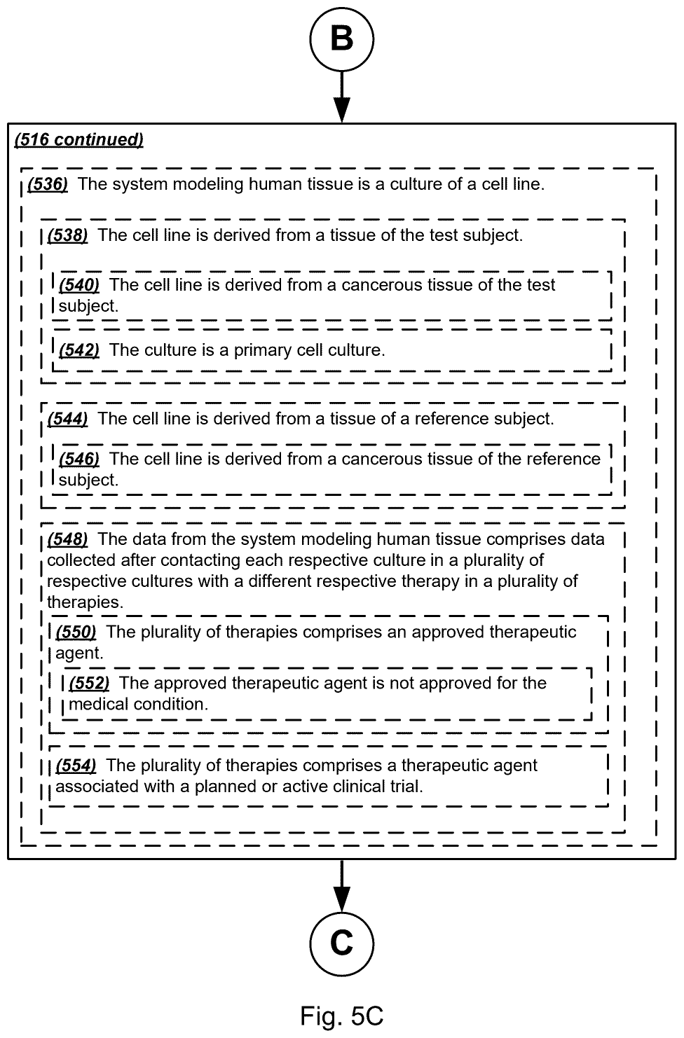

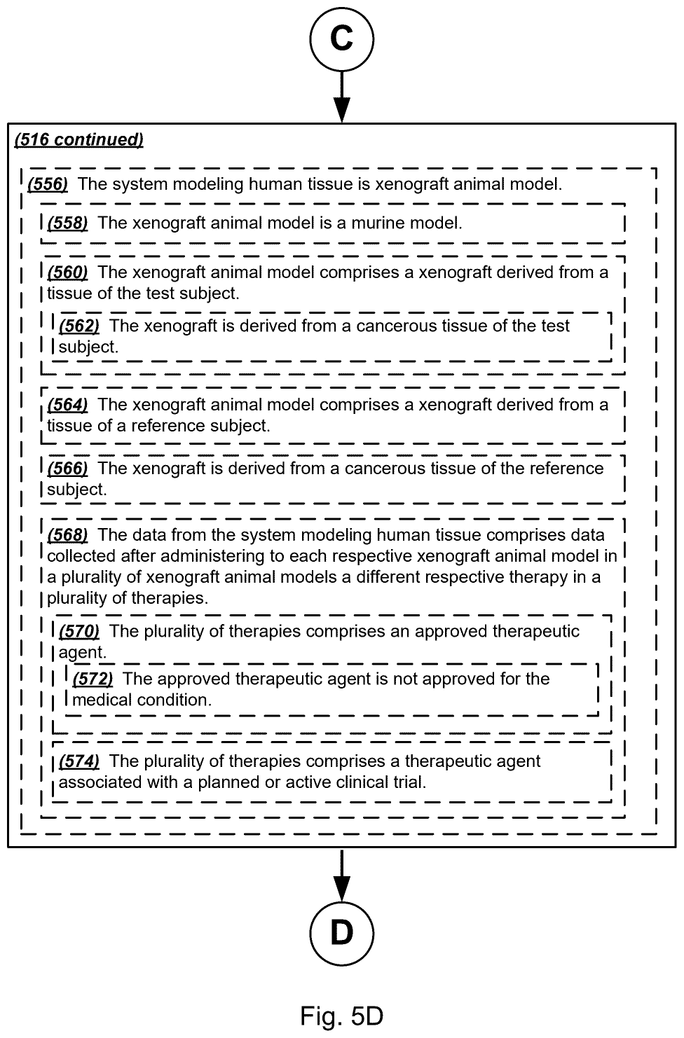

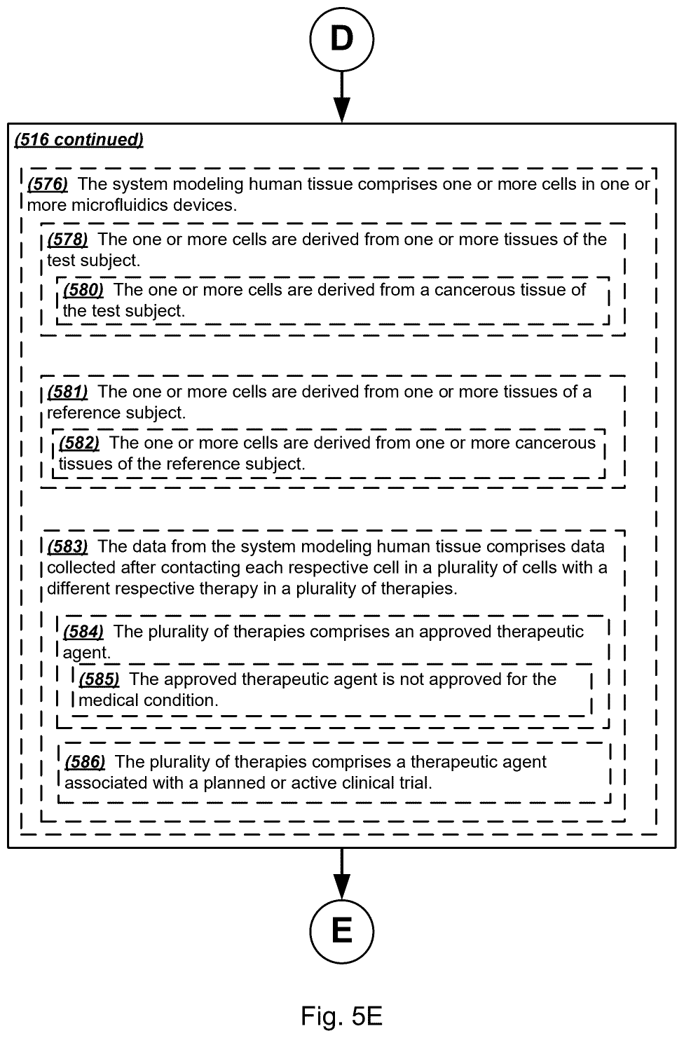

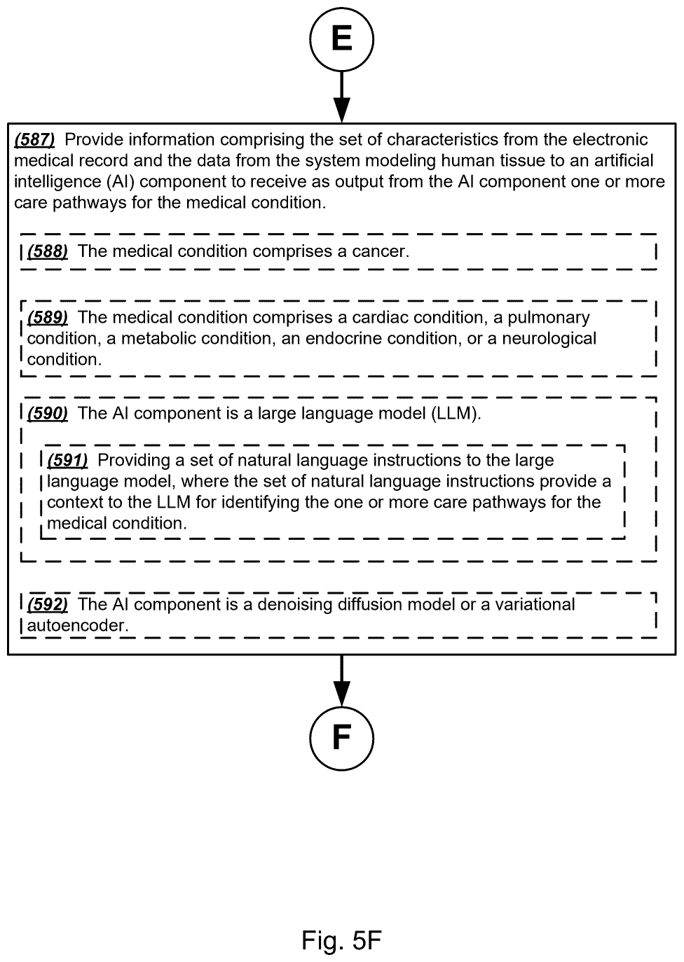

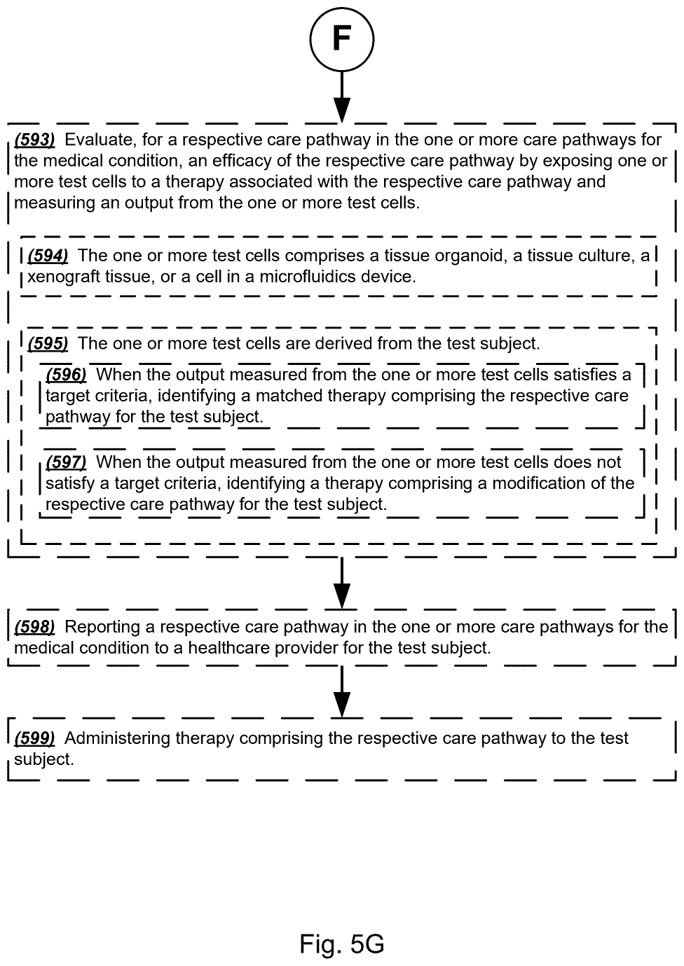

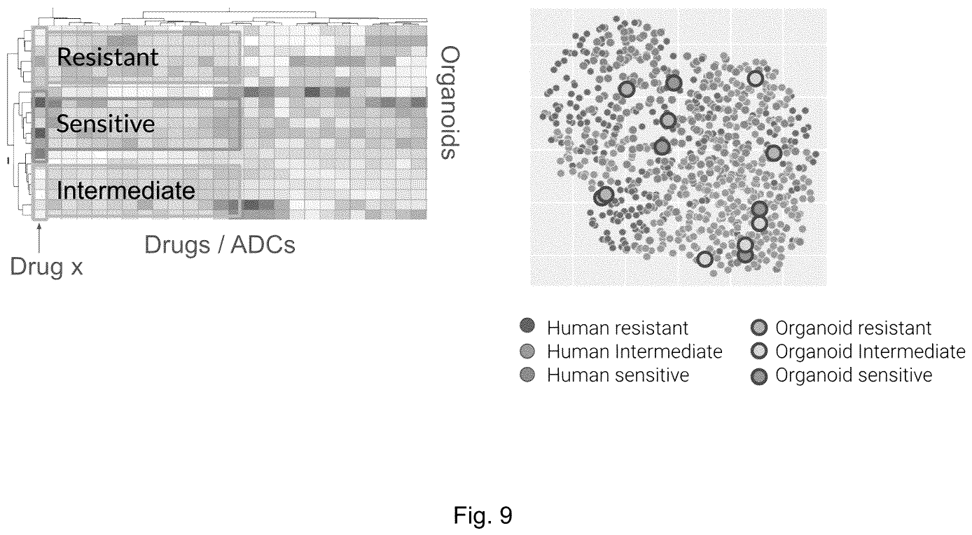

An overview of methods for providing clinical support for personalized medicine is described above with reference to FIGS. 2-4 above. Below, systems and methods for identifying care pathways for treating a disorder in a subject by combining tissue modeling data and electronic health records are described with reference to FIGS. 5-9.

As described herein, in some embodiments, the methods described herein (e.g., methods 500, 600, 700, and 800 as illustrated in FIGS. 5-8) include one or more data collection steps, in addition to data analysis and downstream steps. For example, as described herein, e.g., with reference to FIGS. 2 and 3, in some embodiments, the methods include collection of a biopsy sample and, optionally, one or more matching biological samples from the subject (e.g., a matched cancerous and/or matched non-cancerous sample from the subject). Likewise, as described herein, e.g., with reference to FIGS. 2 and 3, in some embodiments, the methods include extraction of DNA from the biopsy sample (cfDNA) and, optionally, one or more matching biological samples from the subject (e.g., a matched cancerous and/or matched non-cancerous sample from the subject). Similarly, as herein, e.g., with reference to FIGS. 2 and 3, in some embodiments, the methods include nucleic acid sequencing of DNA from the biopsy sample and, optionally, one or more matching biological samples from the subject (e.g., a matched cancerous and/or matched non-cancerous sample from the subject).

However, in other embodiments, the methods described herein include obtaining nucleic acid sequencing results, e.g., raw or collapsed sequence reads of DNA from a biopsy sample (cfDNA) and, optionally, one or more matching biological samples from the subject (e.g., a matched cancerous and/or matched non-cancerous sample from the subject). For example, in some embodiments, sequencing data for a patient is accessed and/or downloaded over network 105 by system 100.

FIGS. 5A-5G collectively provide a flow chart of processes and features for predicting care pathway options for a medical condition in a test subject, in accordance with some embodiments of the present disclosure, embodied as example method 500. Method 500 predicts care pathway options for a medical condition in a test subject. In some embodiments, all or a portion of method 500 is performed at a computer system 100.

Block 502. Referring to block 502, in some embodiments, the method includes retrieving a set of characteristics of the test subject from an electronic medical record for the test subject. Electronic medical records, also referred to as electronic health records (EHRs), provide a useful resource for individualized health data, by combining huge amounts of patient-specific data collected during the provision of healthcare services. For instance, EHRs can include characteristics such as one or more of clinical, demographic, administrative, claims (e.g., medical and pharmacy), and patient-centered (e.g., vital statistics or quality-of-life information obtained from medical instruments or caregiver assessments). Examples of characteristics that can be included in EHRs include medication history (e.g., current prescriptions, concomitant medications, medication classes, medication codes, and/or ontological terms), disease conditions (e.g., pre-existing conditions, co-morbidities, symptoms, diagnoses, and/or prognoses), laboratory test results or clinical data (e.g., biomarkers, genomic variants, medical images, and/or sequencing data), and free-text observations and other notes (e.g., by a clinician). In some instances, EHRs include characteristics in the form of longitudinal data, such as information collected over multiple visits to a healthcare provider or over a period of time. See, for example, Coorevits et al., “Electronic health records: new opportunities for clinical research.” J Intern Med. 2013; 274 (6): 547-560; Cowie et al., “Electronic health records to facilitate clinical research.” Clin Res Cardiol. 2017; 106 (1): 1-9; and Xiao et al., “Opportunities and challenges in developing deep learning models using electronic health records data: a systematic review.” J Am Med Inform Assoc. 2018; 25 (10): 1419-1428, each of which is hereby incorporated herein by reference in its entirety.

In some implementations, EHRs are useful for obtaining information for clinical trials, such as to evaluate study feasibility, coordinate subject recruitment and enrollment, and facilitate pre- and post-trial data collection. In particular, EHR data is useful for pre-screening subjects for eligibility in clinical research (e.g., by age, gender, diagnosis, medications, biomarkers, and/or other demographic or health-related factors). Similarly, EHR data can be used to exclude ineligible subjects, thus reducing overall screening burden for clinical trials, misallocation of trial resources, and the potentially harmful effects of enrolling an ineligible subject in a study. Other non-limiting applications for EHR data include observational studies, safety surveillance, clinical research, and/or regulatory purposes. See, for example, Cowie et al., “Electronic health records to facilitate clinical research.” Clin Res Cardiol. 2017; 106 (1): 1-9, which is hereby incorporated herein by reference in its entirety.

In some embodiments, the EHR is obtained from an image of a health record, such as a scanned image of a physical (e.g., paper and/or handwritten) health record document. In some embodiments, the EHR is obtained in an image file format, such as a PDF. In some such embodiments, the method includes analyzing the EHR, using a text recognition process, to convert the image to computer readable text. For instance, in some embodiments, the method includes, prior to the receiving the EHR, receiving one or more images of text corresponding to the EHR and converting the images to a computer-readable text through text recognition. In some embodiments, the text recognition is optical character recognition (OCR). In some embodiments, the OCR converts the image to raw text.

Methods for text recognition are well known in the art, including but not limited to sliding window classification, Connected Component Analysis (CCA), bounding box regression-based methods, segmentation-based methods, and/or combinations thereof. For example, sliding window classification utilizes convolutional classifiers to detect characters in an image using a multi-scale sliding window. CCA-based methods operate by segmenting pixels in an image having consistent local characteristics such as color, edge, texture, and/or stroke width into characters. Alternatively or additionally, text-line based methods operate by initially identifying lines of text and partitioning the identified text lines into smaller components such as words and letters. Generally, bounding box and segmentation-based methods operate by detecting text at the single-word level, for instance, using bounding boxes that isolate regions of text from the local background, with optional filtering, cleaning, and recognition post-processing. See, for example, Keerthana et al., 2020, “Text Detection and Recognition: A Review,” IRJET Vol 7 (8), 2156-2169, which is hereby incorporated herein by reference in its entirety.

In various embodiments, EHR of a test subject may comprise a comprehensive data set containing a plurality of characteristics. In some embodiments the EHR comprises at least 2, 3, 4, 5, 6, 7, 8, 9, 10, 100, 1,000, 10,000, 100,000 or 1×10⁶ different or more characteristics, either concurrently or cumulatively collected over time. In some embodiments the EHR comprises between 100 and 1×10⁶ different characteristics, between 500 and 1×10⁷ characteristics, or between 1000 and 100, 000 characteristics. The characteristics may encompass, without limitation, physiological data (e.g., heart rate, blood pressure, blood glucose levels, respiratory rate, blood oxygen saturation, body temperature, ECG waveforms), behavioral data (e.g., sleep quality, movement patterns, medication adherence, cognitive task performance), demographic information (e.g., age, sex, height, weight, ethnicity), medical history (e.g., diagnoses, allergies, surgical procedures, immunization records), and lifestyle factors (e.g., tobacco use, alcohol consumption, dietary habits, exercise frequency).

The EHR may further include characteristics in the form of laboratory test results (e.g., complete blood count, liver function, lipid panels), imaging findings (e.g., radiology reports, MRI or CT scan data), treatment plans, and medication histories (e.g., active prescriptions, dosages, refill history). In some embodiments, the characteristics are recorded as single instances or as time-series data, enabling longitudinal analysis. In certain implementations, the EHR may store characteristics collected or derived over time intervals ranging from minutes to multiple years, including but not limited to hourly measurements, daily logs, weekly summaries, monthly trends, or annual check-up data. For example, blood pressure readings may be logged multiple times per day over several weeks, while body weight trends may be captured weekly over several months or years.

The data in the EHR may support dynamic ranges depending on the characteristic type—for instance, heart rate values from 30 to 220 bpm, glucose levels from 50 to 500 mg/dL, sleep durations from 0.5 to 12 hours per night, and cognitive response times from 100 to 5000 milliseconds. The number and type of characteristics stored may be application-specific, and may vary based on clinical protocols, regulatory requirements, user preferences, or the capabilities of data acquisition systems (e.g., wearable devices, remote monitoring platforms, in-clinic diagnostics). These characteristics may be structured to support real-time analytics, trend visualization, decision support, or integration with external healthcare information systems.

Block 504. Referring to block 504, in some embodiments, retrieving the set of characteristics from the electronic health record (EHR) comprises retrieving structured data from the EHR. Structured data may include information that is organized in a standardized format and stored in discrete, machine-readable fields within databases, data tables, or records. This structured data may adhere to established healthcare data models or interoperability standards such as HL7 (Health Level 7), FHIR (Fast Healthcare Interoperability Resources), SNOMED CT (Systematized Nomenclature of Medicine-Clinical Terms), LOINC (Logical Observation Identifiers Names and Codes), ICD (International Classification of Diseases), or CPT (Current Procedural Terminology).

Examples of structured data that may be retrieved include numerical lab results (e.g., blood glucose levels, hemoglobin counts, electrolyte panels), coded diagnoses (e.g., ICD-10 codes), medication lists with standardized drug identifiers (e.g., RxNorm codes), procedure codes, vital signs (e.g., temperature, pulse rate, respiratory rate, blood pressure), and timestamped events such as encounter dates, immunization records, and test result dates. Structured behavioral data may include sleep duration, physical activity metrics, step counts, or cognitive task performance scores, especially when recorded via integrated wearable or remote monitoring devices.

In some embodiments, structured data may further include longitudinal entries that are timestamped and recorded over time, enabling trend analysis and temporal correlation. For example, a test subject's structured record may contain hundreds or thousands of blood pressure readings stored as time-stamped tuples over several months, or daily glucose measurements recorded across multiple years. The retrieval process at block 504 may involve accessing one or more data repositories, clinical data warehouses, or health information exchanges (collectively denoted electronic health record herein) to obtain this structured data. By leveraging structured data, the system may support automated parsing, rule-based filtering, normalization, and data aggregation.

Block 506. Referring to block 506, in some embodiments, retrieving the set of characteristics from the electronic medical record (EMR) comprises retrieving unstructured data contained within the EMR. Unstructured data refers to clinical information that is not stored in predefined fields or standardized formats but rather exists in free-text form, such as physician notes, radiology reports, pathology narratives, discharge summaries, operative reports, and consultation documentation. These data elements often contain rich clinical context, temporal relationships, and nuanced language that are not captured in structured data fields like lab results or medication lists. In some embodiments, natural language processing (NLP) techniques are employed to extract relevant clinical features—such as symptom descriptions, disease staging, patient history, or treatment responses—from these unstructured text sources. Advanced methods, including named entity recognition, clinical concept normalization, and sentiment or temporal analysis, may be applied to identify and interpret medically meaningful content. By incorporating unstructured data into the set of characteristics, the system gains a more complete and accurate picture of the patient's condition, thereby enhancing the quality and relevance of downstream analyses, such as care pathway generation or therapeutic matching.

Block 508. Referring to block 508, in some embodiments, retrieving the set of characteristics from the electronic medical record comprises evaluating at least three data types selected from structured electronic health record (EHR) data, unstructured EHR data, laboratory results, prescribed medications, and performed medical procedures.

In some embodiments, retrieving the set of characteristics from the electronic medical record (EMR) comprises evaluating at least three distinct data types to construct a comprehensive patient profile that informs subsequent clinical analysis or decision-making processes. These data types may be selected from a group that includes: structured EHR data, such as diagnosis codes (e.g., ICD-10), vital signs, problem lists, and coded clinical observations; unstructured EHR data, such as free-text clinical notes, imaging reports, and narrative summaries, which often contain nuanced clinical insights not captured in structured formats; laboratory results, including standard blood panels, molecular diagnostics, biomarkers, and pathology reports, which provide objective, quantitative data on the patient's biological state; prescribed medications, which indicate ongoing and past therapeutic interventions, dosage regimens, and potential drug-drug interactions; and performed medical procedures, such as surgeries, diagnostic imaging, biopsies, or interventional treatments, which reflect both the severity and history of the medical condition.

In some embodiments, sophisticated data parsing and integration tools, such as natural language processing (NLP), clinical ontologies, and interoperability standards (e.g., FHIR or HL7), are employed to extract, normalize, and reconcile these data sources into a unified and interpretable format. By requiring the inclusion of at least three of these modalities, the method ensures a multifaceted and context-rich understanding of the patient's health status, treatment history, and disease progression. This integrated dataset can then be used as input for downstream processes such as AI-driven care pathway generation, eligibility assessment for clinical trials, or functional therapy testing, thereby enhancing the personalization and accuracy of medical decision support.

Block 510. Referring to block 510, in some embodiments, the laboratory results comprises histology data, medical imaging data, genomic sequencing data, transcriptomic data, proteomic data, phlebotomy data, vital signs, or anthropometric data. Accordingly, the laboratory results may comprise a variety of diagnostic and clinical data obtained from biological sampling, imaging, and molecular profiling procedures. The laboratory results may include, for example, histology data such as tissue structure analyses, slide-based pathology findings, or digitized microscopic images annotated by clinical pathologists. Medical imaging data may include raw or processed images, as well as quantitative measurements, from imaging modalities such as magnetic resonance imaging (MRI), computed tomography (CT), ultrasound, or positron emission tomography (PET).

In some embodiments, laboratory results may also include genomic sequencing data derived from whole genome sequencing, whole exome sequencing, or targeted gene panels. Such data may include raw nucleotide sequences, aligned sequence data, and annotated variant call files (VCFs) identifying genetic alterations such as single nucleotide variants (SNVs), insertions or deletions (indels), or copy number variations (CNVs). Transcriptomic data may include gene expression profiles generated from ribonucleic acid sequencing (RNA-seq) or microarray platforms, providing insights into gene activity across different tissues or time points.

Proteomic data may encompass protein abundance measurements, peptide fragment data, and protein modification profiles generated through mass spectrometry or immunoassay techniques. Phlebotomy data may include the results of clinical blood draws, such as hematological panel values, collection timestamps, specimen identifiers, and laboratory accession numbers. The laboratory results may further include vital signs such as heart rate, respiratory rate, blood pressure, body temperature, and blood oxygen saturation, captured either during clinical visits or through continuous monitoring devices. Anthropometric data may include physical body measurements such as height, weight, body mass index (BMI), waist circumference, and body surface area.

In some embodiments, the laboratory results may be collected longitudinally, encompassing repeated measurements or assessments taken over hours, days, weeks, months, or years.

Blocks 512-514. Referring to block 512, in some embodiments, retrieving the set of characteristics from the electronic health record (EHR) comprises inputting all or a portion of the electronic medical record into a large language model (LLM). The LLM may be configured to process unstructured and semi-structured clinical data including physician notes, discharge summaries, diagnostic reports, and other narrative text entries commonly found within the EHR. By leveraging the natural language processing capabilities of the LLM, the system may extract relevant clinical features, temporal information, and relationships between data elements that are otherwise difficult to capture through conventional structured data queries.

For instance, referring to block 514, in some instances, a set of natural language instructions or prompts are provided to the LLM to establish context and guide its analysis. These natural language instructions may include task-specific directives, such as identifying characteristics related to a particular medical condition, disease state, or symptom set associated with the test subject. The natural language instructions may specify the desired output format, level of detail, or focus areas. For example, natural language instructions may instruct the LLM to extract all references of biomarkers, relevant laboratory values, treatment histories, or symptom descriptions linked to the medical condition.

The LLM may also utilize domain-specific ontologies, medical vocabularies, and semantic embeddings to improve its understanding of clinical terminology and context, enabling accurate disambiguation of complex medical language. Through this approach, the LLM can effectively parse diverse clinical narratives, recognize negations or temporal qualifiers (e.g., “no history of diabetes,” “symptoms resolved”), and aggregate findings across multiple documents or visits.

In some embodiments, the output from the LLM is a structured representation of the set of characteristics, such as key-value pairs, coded medical concepts, or annotated text segments. Additionally, the system 100 may iteratively refine natural language instructions or leverage active learning techniques to enhance HER extraction accuracy over time.

Block 516. Referring to block 516, in some embodiments, retrieving the set of characteristics from the electronic health record (EHR) comprises retrieving information from a database that has been populated or derived from the EHR. Such a database may be designed to aggregate, normalize, and index relevant clinical data extracted from multiple sources within the EHR, including structured fields, unstructured text, laboratory results, imaging metadata, and other ancillary records.

In some implementations, the electronic medical records may be continuously or periodically processed using automated extraction pipelines, natural language processing (NLP) tools, and/or data normalization algorithms to identify and curate pertinent health information. In some embodiment this curated data is then stored within a curated database, which may support indexing and optimized query capabilities, such as relational database management systems (RDBMS), NoSQL databases, or specialized clinical data repositories designed for rapid retrieval.

By maintaining an up-to-date, pre-processed curated database, the system 100 can significantly improve the efficiency of downstream analyses and computational workflows. This approach reduces the latency associated with on-demand processing of raw EHRs, enabling faster access to test subject characteristics.

Furthermore, the curated database may incorporate mechanisms for version control, audit logging, and data provenance tracking to ensure data integrity, reproducibility, and compliance with regulatory standards such as the Health Insurance Portability and Accountability Act (HIPAA) or the General Data Protection Regulation (GDPR). In some embodiments the curated database, in the form of a pre-populated, indexed database derived from one or more test subject EHRs provides a scalable and efficient foundation for retrieving complex, multi-dimensional health characteristics while minimizing computational overhead and improving system responsiveness and efficiency.

Block 517. Referring to block 517, the method also includes retrieving data from a system modeling human tissue. In some embodiments, the system modeling human tissue is a cell or tissue culture. In other embodiments, the system modeling human tissue is an in silico model. For example, U.S. Patent Publication No. 2025-0087317 A1, entitled “Predicting Unobserved Quantitative Measures Using Machine Learning” and hereby incorporated by reference, describes example methods for simulating experimental data based on database of existing experimental data or freshly generated experimental data. In some embodiments, the methods described therein can simulate results of intermediate drug doses without actually testing those doses, or can simulate intermediate time points that were not measured in an experiment.

Continuing to refer to block 517, in some embodiments the method includes retrieving data from a system that models human tissue. In some embodiments, this is in addition to the retrieval of characteristics from the EHR. In some embodiments the system modeling human tissue serves as an independent and complementary source of biological insight, enabling the method to incorporate mechanistic or experimental data that may not be found in the EHR. In some embodiments, for the sake of illustration, the data from a system that models human tissue is represented as being stored in the test subject human modeling record in FIG. 1 independent of the EHR. However, it will be appreciated that in other embodiments, the test subject human modeling record is combined with EHR characteristics in test subject data store 120.

In some embodiments, the system modeling human tissue comprises one or more biological models such as cell cultures, organoids, organ-on-a-chip devices, or ex vivo tissue explants. These models are engineered or selected to replicate specific human tissue architecture, molecular expression patterns, or pathological states. They may be derived from primary human cells, induced pluripotent stem cells (iPSCs), or immortalized cell lines, and can be exposed to defined perturbations, such as pharmaceutical compounds or genetic modifications, to elicit measurable physiological or biochemical responses. The data retrieved from these systems may include gene expression profiles, protein markers, cell viability metrics, or metabolic outputs, among others.

In some embodiments, the system modeling human tissue additionally or alternatively comprises one or more in silico models of the test subject's tissue, implemented as a software-based simulation environment. These computational models may include physiologically based pharmacokinetic (PBPK) models, systems biology simulations, agent-based models, or machine learning-driven virtual tissue constructs. These in silico models may be parameterized using both historical and real-time data and can simulate biological outcomes under untested conditions. For example, U.S. Patent Publication No. 2025-0087317 A1, entitled “Predicting Unobserved Quantitative Measures Using Machine Learning” and hereby incorporated by reference in its entirety, describes methods for generating synthetic or interpolated experimental data. These may include predictions for intermediate drug doses, unsampled time points, or unmeasured biomarkers based on existing experimental datasets.

Retrieving data from a system modeling human tissue enables access to biologically plausible, experimentally or computationally derived insights that complement and extend the clinical and observational data available in the EHR. This multimodal integration enhances the method's ability to generate comprehensive, mechanistically informed patient profiles. The inclusion of tissue-model-derived data ensures that the method incorporates both real-world patient data and high-fidelity experimental or simulated data sources, increasing robustness, accuracy, and translational relevance.

Block 518. Referring to block 518, in some embodiments, the system modeling human tissue comprises an organoid culture and the test subject human modeling record includes organoid culture test results 130 from such organoids. An example method for culturing tumor organoids is described in U.S. Pat. No. 11,629,385, the content of which is incorporated herein by reference in its entirety. In some embodiments, an organoid, e.g., a tumor organoid, is co-cultured with one or more immune or effector cells. In some embodiments, the one or more immune or effector cells are derived from the same patient as is the organoid (e.g., the tumor organoid). Example methods for co-culturing organoids and immune or effector cells are described in U.S. Patent Application Publication No. 2023/0036156, the content of which is incorporated herein by reference in its entirety.

Blocks 520-522. Referring to block 520, in some embodiments, the organoid culture comprises organoids derived from a tissue of the test subject. Referring to block 522, in some embodiments, the organoid culture comprises tumor organoids derived from a cancerous tissue of the test subject. In some embodiments, the test subject-derived organoids are co-cultured with immune or effector cells from the same patient, in order to better model the tumor microenvironment.

In some such embodiments, these organoids are generated specifically to evaluate oncological conditions, such as solid tumors or metastatic lesions. The organoids may be established from biopsy or surgical resection specimens obtained from the test subject's tumor, enabling ex vivo modeling of the subject's specific cancer phenotype. These tumor-derived organoids preserve key molecular and histological characteristics of the original malignancy, including genomic mutations, epigenetic profiles, cellular heterogeneity, and microenvironmental interactions.

In some embodiments, the organoids may be derived from a variety of tissue types, either healthy or cancerous (tumor), including but not limited to colorectal, pancreatic, breast, lung, liver, kidney, ovarian, prostate, gastric, or brain tissues. The organoids may also represent subtypes within cancers from such origins (e.g., triple-negative breast cancer, KRAS-mutant colorectal cancer, EGFR-mutant non-small cell lung cancer), and may retain the tumor's sensitivity or resistance to specific classes of therapies. In some embodiments these organoids are cultured in 3D matrices such as extracellular matrix hydrogels under conditions that support their growth and differentiation, including the use of tumor-specific media enriched with growth factors, signaling modulators, or niche-supporting co-factors.

In various embodiments, a plurality of organoids, ranging from one to thousands, are cultured in a high-throughput or semi-automated format, such as in multiwell plates or microfluidic arrays. These organoids may then be exposed to one or more classes of anti-cancer agents, including cytotoxic chemotherapies (e.g., platinum compounds, taxanes), targeted therapies (e.g., tyrosine kinase inhibitors, monoclonal antibodies), hormone therapies, immunotherapies (e.g., checkpoint inhibitors), or investigational agents under preclinical evaluation. In some embodiments, drug testing includes both single-agent and combinatorial regimens, with variable concentrations and exposure durations to simulate clinically relevant dosing schedules.

Organoid response to drug treatment may be assessed using a range of readouts, including but not limited to (i) cell viability assays (e.g., ATP-based luminescence, live/dead staining), apoptosis or proliferation markers (e.g., cleaved caspase-3, Ki-67), transcriptomic changes (e.g., via RNA sequencing or qPCR), morphological alterations captured through high-content imaging, resistance signatures or pathway activation profiles (e.g., phospho-protein analysis, immunofluorescence).

In some embodiments, organoids are used to determine the susceptibility or resistance of the test subject's tumor to a given therapeutic class, based on empirically observed response patterns. The resulting data may indicate drug efficacy, partial resistance, or complete refractoriness, and may guide the selection of personalized treatment strategies, including avoidance of ineffective agents or identification of alternative, more responsive regimens.

The integration of organoid-derived drug response data with other patient-specific information (e.g., characteristics of electronic health records) enables a comprehensive, biologically grounded framework for clinical decision-making. This approach enhances the predictive value of treatment selection and supports adaptive oncology strategies, such as switching therapies upon detection of acquired resistance. Because the organoids are derived directly from the test subject, they provide a highly individualized model system that captures dynamic, tumor-intrinsic responses and may be re-cultured or re-tested at multiple time points during disease progression or following therapeutic intervention.

Block 524. Referring to block 524, in some embodiments, the organoid culture comprises organoids derived from a tissue of a reference subject. In some embodiments, the organoids are derived not from a single individual but from a cohort of reference subjects, which may include healthy individuals selected to represent a normative biological baseline. In some embodiments the cohort of reference subjects is 2, 3, 4, 5, 6, 7, 8, 9, 10, 11, 12, 13, 14, 15, 16, 17, 18, 19, 20 or more different reference subjects. In some embodiments the cohort of reference subjects is 20, 30, 40, 50, 60, 70, 80, 90, 100, 110, 120, 130, 140, 150, 160, 170, 180, 190, or 200 different reference subjects. In some embodiments such reference subjects are healthy. In some embodiments the cohort subjects represent a broad array of different tissues, such as 5 or more, 10 or more, 15 or more, or 20 or more different tissues, meaning that for each such respective tissue, there is at least 1, 2, 3, 4, or 5 or more organoids derived from the respective tissue.

The tissue used to generate the organoids may be obtained from elective biopsies, surgical discards, or donor programs, and may include tissue from organs such as the colon, lung, liver, pancreas, kidney, prostate, or breast.

Organoids derived from healthy reference subjects enable systematic comparison between diseased and non-diseased tissues under identical experimental conditions. For example, when evaluating drug effects, such reference organoids provide a control group for determining whether observed cytotoxicity is tumor-specific or reflects general tissue toxicity. They may also help identify off-target effects, reveal differential pathway activation in healthy versus cancerous tissues, or serve as controls for molecular profiling assays such as RNA sequencing or proteomics. Moreover, organoids from a healthy cohort can be stratified by age, sex, ancestry, or other biological factors to assess population-level variability in baseline tissue response.

Block 526. Referring to block 526, in some embodiments, the organoid culture comprises tumor organoids derived from cancerous tissue of a reference subject. These tumor organoids may represent well-characterized cancer subtypes, specific mutational signatures, or established drug resistance profiles that serve as valuable comparators for interpreting the behavior of organoids derived from the test subject. For example, a tumor organoid with known sensitivity to a particular targeted therapy may be used as a positive control, while another organoid with documented resistance mechanisms may help contextualize resistance observed in the test subject's sample.

In some embodiments, the organoid culture includes a combination of organoids from both the test subject and one or more reference subjects, including both healthy and diseased donors. This mixed-origin culture enables intra-experimental normalization and supports high-resolution comparisons across a spectrum of tissue types and biological states. Such configurations are particularly valuable in oncology applications, where distinguishing subject-specific drug response from common or expected patterns across a population is critical for personalized treatment selection.

In some embodiments, one or more organoids are derived from each subject in the cohort of reference subjects. In some embodiments each subject in the cohort of subjects is afflicted with the same medical condition as the test subject. In some embodiments the cohort subjects represent a broad array of different medical conditions, such as 5 or more, 10 or more, 15 or more, or 20 or more different medical conditions. In some embodiments the cohort subjects represent a broad array of different cancers, such as 5 or more, 10 or more, 15 or more, or 20 or more different cancers, meaning that for each such respective cancer, there is at least 1, 2, 3, 4, or 5 or more subjects in the cohort of subjects that have that cancer. Such embodiments provide additional context to the data derived from the organoids from the test subject.

In some embodiments, modeling data are aggregated from multiple organoid-based experiments, potentially across hundreds or thousands of individual samples. The data may include phenotypic, transcriptomic, proteomic, metabolomic, or drug-response measurements, and may be derived from organoids representing a wide range of tissues, disease states, and donor demographics. In various embodiments, the modeling data include data from at least 2, at least 3, at least 4, at least 5, at least 10, at least 25, at least 100, at least 250, at least 500, at least 1,000, at least 5,000, at least 10,000, at least 50,000, at least 100,000, or more different organoids.

The inclusion of organoids from a healthy reference cohort, combined with tumor organoids and subject-specific cultures, in some embodiments enables the construction of comprehensive comparative frameworks. These datasets can be used to benchmark the test subject's biological responses, identify therapeutic vulnerabilities, and detect atypical drug sensitivities or resistance mechanisms. Additionally, such organoid cohorts support training and validation of predictive machine learning models used to forecast treatment outcomes or stratify patients into clinically actionable groups.

Block 528. Referring to block 528, in some embodiments, the data from the system modeling human tissue comprises data collected after contacting each respective organoid culture in a plurality of organoid cultures with a different respective therapy in a plurality of therapies.

In some embodiments, the plurality of therapies includes a small molecule therapy, an antibody therapy, an antibody-drug conjugate (ADC) therapy, a gene therapy, or the like. Example therapy or drug targets include: ataxia telangiectasia and Rad3 related (ATR) kinase, ataxia telangiectasia mutated kinase (ATM), checkpoint kinase 1 (Chk1), checkpoint kinase 2 (Chk2), DNA-PK, cyclin-dependent kinase (CDK), WEE1, poly (ADP-ribose) polymerase (PARP). Example therapy or drug classes include: immuno oncology therapy, chemotherapy, PARP inhibitors (PARPi). Therapy may include one or more of: an inhibitor of SUV4-20 (SUV420H1 or SUV420H2), a tyrosine kinase inhibitor, a retinoid-like compound, a weel kinase inhibitor, an anaplastic lymphoma kinase inhibitor, an aurora A kinase inhibitor, an aurora B kinase inhibitor, a reversible inhibitor of eukaryotic nuclear DNA replication, an antimetabolite antineoplastic agent, an ataxia telangiectasia and Rad3-related protein (ATR) kinase inhibitor, an ATM kinase inhibitor, a checkpoint kinase inhibitor, a GSK-3a/b inhibitor, a proteasome inhibitor, an AXL or RET inhibitor, a c-Met or VEGFR2 inhibitor, an alkylating antineoplastic agent, a DNA-PK and/or mTOR inhibitor, an inhibitor of mammalian target of rapamycin (mTOR), a checkpoint kinase 1 (CHK1) inhibitor, a retinoic acid receptor β (RARβ) or RARγ antagonist, a retinoic acid receptor (RAR) γ-selective agonist, RARγ-selective retinoid, inducer of apoptosis, CDK2 a RAR agonist, a chemotherapy, a tyrosine kinase inhibitor antineoplastic agent, an antimicrotubular antineoplastic agent, a topoisomerase inhibitor antincoplastic agent, a sodium-glucose cotransporter-2/SGLT2 inhibitor, an inhibitor of the tropomyosin receptor kinases A, B and C, C-ros oncogene 1 and anaplastic lymphoma kinase, a topoisomerase inhibitor antineoplastic agent, an inhibitor of mTOR, an inhibitor of phosphatidylinositol 3-kinase (PI3K), an inhibitor of RIP3K, an analog of cyclophosphamide, an SGLT2 inhibitor, aWnt/β-catenin inhibitor, a tyrosine kinase inhibitor that interrupts the HER2/neu and epidermal growth factor receptor/EGFR pathways, an inhibitor of tropomyosin kinase receptors TrkA, TrkB, and TrkC, a cyclin-dependent kinase (CDK) inhibitor, a CDK7 inhibitor, an inhibitor of VEGFR1, VEGFR2 and VEGFR3 kinases, a DNA-PK/PI3K/mTOR inhibitor, a poly ADP ribose polymerase (PARP) inhibitor, an inhibitor of Rac GTPase, a taxane, a Bromodomain And PHD Finger Containing 1 (BRPF1) bromodomain inhibitor, a mitogen-activated protein kinase-activated protein kinase 2 (MAPK2) inhibitor, a RAF inhibitor, a histone deacetylase (HDAC) inhibitor, a CDK1 inhibitor, aTGF-beta/Smad inhibitor, a Pim kinase inhibitor, a DNA topoisomerase I inhibitor, active metabolite of CPT-11/Irinotecan, an atypical retinoid, apoptosis inducer, a multi-kinase inhibitor, a fms-like tyrosine kinase-3 (FLT3) inhibitor, a MEK inhibitor, an inhibitor of extracellular signal-regulated kinase (ERK) 1 and/or 2, or a DNA-dependent protein kinase/DNA-PK inhibitor.

In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies. Example methods for screening potential therapies on tumor organoids are described in U.S. Patent Application Publication No. 2022/0392640, as well as U.S. Pat. Nos. 11,415,571 and 11,561,178, the disclosure of which are each incorporated herein by reference in their entireties.

Block 530. Referring to block 530, in some embodiments, the plurality of therapies comprises at least one therapeutic agent that has been approved for use in humans. In some embodiments, the approved therapeutic agent is specifically approved for use in treating the medical condition being investigated, such as a particular form of cancer, inflammatory disease, neurological disorder, infectious disease, or metabolic condition. Approval, in this context, refers to formal authorization issued by a national or supranational regulatory authority, such as the U.S. Food and Drug Administration (FDA), the European Medicines Agency (EMA), the Pharmaceuticals and Medical Devices Agency (PMDA) in Japan, Health Canada, the Therapeutic Goods Administration (TGA) in Australia, or other analogous bodies, that permits the marketing and clinical use of the therapeutic agent for one or more specified indications.

Such approval is typically granted following a rigorous evaluation of preclinical data, safety studies, and human clinical trial results demonstrating efficacy, safety, and quality. The approved indications for a given therapeutic agent are usually documented in a product's official labeling or Summary of Product Characteristics (SmPC), which outlines the disease(s) or condition(s) for which the agent is authorized, recommended dosages, contraindications, and administration routes. In the context of the present method, a therapeutic agent that is approved for the specific condition under investigation may serve as a reference or benchmark against which the responses of the test subject's organoids are compared.

Block 532. Referring to block 532, in some embodiments, the approved therapeutic agent is not approved for the medical condition being studied. In these cases, the therapy may still be used under what is commonly referred to as off-label use, where a licensed medical product is administered for a disease or condition not explicitly covered by its regulatory approval. Off-label usage may arise when clinical experience, smaller-scale studies, or real-world data support the potential effectiveness of the therapy for a new indication that has not yet undergone formal regulatory review for that specific use.

Off-label use is common in areas of unmet clinical need, such as oncology, rare diseases, or pediatric medicine, where approved therapies may be limited or non-existent. While physicians are generally permitted to prescribe medications off-label based on their clinical judgment, such uses are not typically promoted by manufacturers and may not be reimbursed by insurers unless supported by strong clinical evidence or practice guidelines. In some embodiments, the present method evaluates how test subject-derived organoids respond to such off-label therapies, either confirming or challenging anecdotal evidence or clinical assumptions about the efficacy of these agents in a new disease context.

In further embodiments, the therapeutic agent is neither approved for the condition nor widely recognized as a candidate for off-label use. In this case, the therapy may be considered investigational or experimental, meaning it has not been subject to regulatory evaluation for any formal indication or may still be in preclinical or early-phase clinical trials. The inclusion of such unapproved or unrecognized therapies in the set of tested agents allows the method to identify novel treatment candidates, reposition existing drugs, or generate data to support future clinical investigation. In oncology, for instance, the system may be used to evaluate kinase inhibitors, immunomodulatory agents, or biologics that are approved for one tumor type but show promising activity in another, mechanistically related context.

In all such embodiments, whether the therapy is approved for the condition, approved for other conditions, used off-label, or entirely unapproved, the organoid-based modeling framework described herein allows for empirical evaluation of therapeutic efficacy on a personalized, biologically relevant platform. This facilitates treatment prioritization, risk-benefit analysis, and identification of unexpected vulnerabilities in a test subject's tumor or tissue model, enabling precision medicine.

Block 534. Referring to block 534, in some embodiments, the plurality of therapies comprises a therapeutic agent that is associated with a clinical trial, either planned, active, or recently completed. The clinical trial may be registered with an official trial registry, such as ClinicalTrials.gov, the EU Clinical Trials Register, or similar databases maintained by national regulatory bodies. In various embodiments, the therapy has reached different stages in the clinical trial pipeline, including Phase I (safety and dosage), Phase II (efficacy and side effects), Phase III (comparative effectiveness), or Phase IV (post-marketing surveillance). The inclusion of investigational agents currently under clinical evaluation allows the described method to identify therapeutic options that, while not yet broadly available, may offer a mechanistic or targeted rationale based on the molecular or phenotypic characteristics of the test subject.

In some embodiments, the clinical trial is actively recruiting participants at the time of analysis. Under such circumstances, if the subject-specific data, e.g., organoid response profiles, biomarker expression, or genomic mutations, align with the inclusion criteria of the trial protocol, the test subject may be referred for potential enrollment. The method may be used to flag trials with eligibility criteria that match the test subject's clinical or molecular profile, including rare tumor subtypes, specific gene variants, or treatment-refractory status.

In other embodiments, the method may evaluate responses to therapeutic agents being studied in clinical trials for related but distinct indications. For instance, a drug being trialed for one form of solid tumor may show promising organoid-based activity in a different tumor type present in the test subject. This information may support compassionate use applications, expanded access protocols, or enrollment in basket or umbrella trials that accommodate biomarker-driven stratification across multiple disease types.

In some implementations, data from the modeling system may be used not only to identify eligibility for existing trials, but also to inform trial design, such as identifying underexplored responder subpopulations or prioritizing combinations of therapies for prospective studies. The inclusion of therapies under clinical investigation allows the system to remain responsive to the rapidly evolving oncology and drug development landscape, offering access to potentially beneficial therapies before formal approval.

In yet other embodiments, the modeling results may help determine whether a test subject is likely to benefit from a trial therapy based on empirical, ex vivo response rather than trial eligibility alone. For example, even if the subject meets all inclusion criteria, poor organoid response to the trial agent may warrant reconsideration of enrollment or suggest that an alternative trial would be more appropriate.

Thus, the integration of clinical trial-associated therapies into the set of candidate treatments broadens the clinical and investigational scope of the system and supports adaptive, data-driven therapeutic decision-making for both current and future treatment options.

Block 536. Referring to block 536, in some embodiments, the system modeling human tissue is additionally or alternatively one or more cultures of one or more cell lines. Cell line models provide a robust, reproducible, and experimentally tractable platform for studying human biological responses in vitro. These models may be used to evaluate cellular proliferation, signaling pathway activity, drug sensitivity, metabolic responses, gene expression, or molecular pathway perturbations.

In some embodiments, a plurality of distinct cell lines are used. For example, the plurality of cell lines may include 2, 3, 4, 5, 6, 7, 8, 9, 10, 11, 12, 13, 14, 15, 16, 17, 18, 19, or 20 or more cell lines. In certain embodiments, the number of cell lines ranges between 5 cell lines and 50 cell lines, 10 cell lines and 100 cell lines, 20 cell lines and 200 cell lines. These cell lines may differ by tissue of origin, genetic background, disease subtype, or known resistance profiles. The use of multiple cell lines enables broader screening for treatment responsiveness and allows for evaluation of how therapeutic effects vary across diverse biological contexts.

Block 538. Referring to block 538, in some embodiments, one or more of the cell lines are derived from tissue of the test subject. Test subject-derived cell lines may capture test subject-specific characteristics such as mutations, epigenetic features, or transcriptomic profiles, and may be used to validate findings observed in other model systems such as organoids or in silico predictions.

Block 540. Referring to block 540, in some embodiments, one or more of the cell lines are derived from a cancerous tissue of the test subject. Tumor-derived cell lines may be used to evaluate responses to cytotoxic, targeted, or immunomodulatory agents, and may be particularly useful for longitudinal monitoring of therapeutic resistance or clonal evolution through successive rounds of treatment.

Block 542. Referring to block 542, in some embodiments, the culture comprises primary cell cultures. These may be established directly from freshly resected or biopsied tissue without immortalization and maintained for a limited duration to preserve in vivo-like behavior. In some embodiments, combinations of primary and immortalized cell lines may be used in parallel to compare stable, scalable in vitro models with short-term, high-fidelity representations of patient biology.

In embodiments using multiple cell lines, data may be collected under standardized or perturbed conditions, with or without therapeutic agents, and subjected to comparative analysis across the full set of lines. This approach supports both individualized therapeutic modeling for the test subject and broader cohort-level or population-level inferences about treatment efficacy, drug resistance, or molecular mechanism of action.

Block 544. Referring to block 544, in some embodiments, a cell line is derived from a tissue of a reference subject. The reference subject may be a healthy donor or an individual with a well-characterized clinical and molecular background. In some embodiments, the cell lines are derived from a cohort of reference subjects, including individuals selected to reflect a range of demographic or genetic variables such as age, sex, ancestry, or known polymorphisms. The cell lines may be obtained from established biobanks, commercial cell repositories, or newly derived from donor tissue, including but not limited to skin, lung, gastrointestinal tract, kidney, liver, or other organ systems.

Reference-derived cell lines may serve multiple roles, such as providing a normative baseline for evaluating biological effects, controlling for assay variation, or enabling comparative modeling of disease versus non-disease states. For example, when evaluating the cytotoxic or molecular effects of a given therapy in a test subject's tumor-derived cell line, the inclusion of reference cell lines from healthy donors allows the identification of effects that are tumor-selective versus broadly cytotoxic. Moreover, data from reference cell lines may be used to calibrate high-throughput screens, normalize molecular readouts, or establish population-level thresholds for treatment response.

Block 546. Referring to block 546, in some embodiments, the one or more cell lines is derived from a cancerous tissue of a reference subject. These cancer-derived reference lines may represent diverse tumor types, molecular subtypes, or resistance phenotypes, and may be drawn from publicly available cell line panels (e.g., NCI-60, CCLE, GDSC) or from proprietary or newly established repositories. In some embodiments, such cell lines are derived from tumors carrying specific oncogenic alterations, such as KRAS, EGFR, BRAF, or TP53 mutations, or from cancers known to be resistant or sensitive to particular classes of therapies, including chemotherapy, small-molecule inhibitors, or immune checkpoint modulators.

By including cancer cell lines from reference subjects with known response profiles or defined molecular features, the system supports benchmarking of the test subject's therapeutic response in a broader biological and clinical context. For instance, the test subject's tumor-derived cell line may be analyzed alongside reference tumor cell lines known to be responsive to a particular therapy. If the test subject's line displays similar response behavior, this may provide additional confidence in therapeutic selection. Conversely, if the test subject's line deviates from known patterns, the divergence may point to unique features or emerging resistance mechanisms.

In some embodiments, a plurality of reference-derived cancer cell lines is used, including 2, 3, 4, 5, or 10 or more distinct lines, or ranging from 10 to 100, 20 to 200, or even more than 500 lines. In some such embodiments, each such cell line is from a different subject in the cohort of reference subjects. In some such embodiments, more than 1 such cell line is derived from the same reference subject in the cohort of reference subjects.

These reference cancer cell lines may support population-scale analyses of treatment heterogeneity, help identify biomarker-treatment associations, or be used to train machine learning models to predict drug efficacy based on cell line profiles.

In all such embodiments, the inclusion of reference-derived cell lines—both healthy and cancerous—provides experimental context, supports comparative modeling, and enhances the robustness and interpretability of results generated by the system for a given test subject.

Block 548. Referring to block 548, in some embodiments, the data from the system modeling human tissue comprises data collected after contacting each respective culture in a plurality of respective cultures with a different respective therapy in a plurality of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 550-554. Referring to blocks 550-554, in some embodiments, the plurality of therapies comprises any of the therapies disclosed in block 530 and/or block 532 and/or block 534.

Block 556. Referring to block 556, in some embodiments, the system modeling human tissue is additionally or alternatively a xenograft animal model. Xenograft models provide a robust, reproducible, and experimentally tractable in vivo platform for studying human biological responses. These models may be used to evaluate tumor growth kinetics, metastatic potential, signaling pathway activity, therapeutic response, pharmacodynamics, immune interactions, or other molecular and cellular perturbations under physiologic conditions.

In some embodiments, a plurality of distinct xenograft models is used. For example, the plurality of xenograft models may include 2, 3, 4, 5, 6, 7, 8, 9, 10, 11, 12, 13, 14, 15, 16, 17, 18, 19, or 20 or more xenograft models. In certain embodiments, the number of xenograft models ranges between 5 and 50, 10 and 100, 20 and 200, or more. These xenograft models may differ by tissue of origin, genetic background, disease subtype, tumor microenvironment features, or known treatment resistance profiles. The use of multiple xenograft models enables broader evaluation of therapeutic effects and allows for studying response variability across diverse biological and clinical contexts.

Block 558. Referring to block 558, in some embodiments, the xenograft animal model is a murine model.

Block 560. Referring to block 560, in some embodiments, one or more of the xenograft models are established using tissue derived from the test subject. Subject-derived xenograft models may capture individual-specific characteristics such as somatic mutations, gene expression signatures, immune landscape, or epigenetic features. These personalized in vivo models may be used to validate results from other platforms, such as organoid systems or computational simulations, and may directly inform individualized therapeutic decision-making.

Block 562. Referring to block 562, in some embodiments, one or more of the xenograft models are derived from cancerous tissue of the test subject. Tumor-derived xenograft models (e.g., patient-derived xenografts or PDXs) allow for preclinical testing of anticancer agents within an in vivo system that recapitulates the cellular architecture and biological complexity of the subject's tumor. These models may be used to assess drug efficacy, predict resistance, monitor tumor progression, or identify synergistic treatment combinations over time.

Block 564. Referring to block 564, in some embodiments, the xenograft model is derived from a tissue of a reference subject. The reference subject may be a healthy donor or an individual with a well-characterized clinical and molecular profile. In some embodiments, the system includes xenograft models derived from a cohort of reference subjects selected to represent diversity across sex, age, ancestry, or known genetic polymorphisms. Tissues may be sourced from repositories, clinical samples, or newly acquired specimens and engrafted into appropriate immunocompromised or humanized animal hosts for in vivo study.

Reference-subject-derived xenograft models may serve multiple roles, such as establishing normative baselines for comparison, controlling for biological variability, or providing context for evaluating tumor-specific responses. For instance, when evaluating the therapeutic response of a test subject's tumor-derived xenograft, parallel evaluation of xenografts from healthy or non-diseased tissues may reveal tumor-specific vulnerabilities versus general systemic effects. Reference xenografts also aid in calibrating assay sensitivity and in interpreting treatment effects across different biological backgrounds.

Block 566. Referring to block 566, in some embodiments, the xenograft model is derived from a cancerous tissue of a reference subject. These cancer-derived reference xenografts may represent diverse tumor types, molecular subtypes, or clinically relevant treatment profiles. In some embodiments, the tumors used for engraftment harbor defined genetic alterations such as TP53, EGFR, BRAF, KRAS, or ALK mutations, or are selected based on known responsiveness or resistance to classes of therapies including chemotherapy, targeted small molecules, antibody therapies, or immunotherapies.

Block 568. Referring to block 568, in some embodiments, the data from the system modeling human tissue comprises data collected after administering to each respective xenograft animal model in a plurality of xenograft animal models a different respective therapy in a plurality of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 570-574. Referring to blocks 570-574, in some embodiments, the plurality of therapies comprises any of the therapies disclosed in block 530 and/or block 532 and/or block 534.

Block 576. Referring to block 576, in some embodiments, the system modeling human tissue additionally or alternatively comprises one or cells in one or more microfluidics devices. In some embodiments, each such microfluidics device is a lab on a chip. For a review of lab on a chip technology see, for example, Lab Chip (23) (2023), the entire edition of which is dedicated to lab on a chip review articles, and of which the contents of the edition are incorporated herein by reference in its entirety. In some embodiments, individual cells are evaluated with the one or more microfluidics devices. In other embodiments, organoids, e.g., tumor organoids, are evaluated with the one or more microfluidic devices.

In some embodiments, the one or more cells may include 2, 3, 4, 5, 6, 7, 8, 9, 10, 11, 12, 13, 14, 15, 16, 17, 18, 19, or 20 or more cells. In certain embodiments, the number of cells ranges between 5 cells and 50 cells, 10 cells and 100 cells, 20 cells and 200 cells. In some embodiments, the number of cells is more than 100 cells, 1000 cells, 10,000 cells, 100,000 cells, or 1×10⁶ cells. These cells may differ by tissue of origin, genetic background, disease subtype, or known resistance profiles. The use of multiple cells enables broader screening for treatment responsiveness and allows for evaluation of how therapeutic effects vary across diverse biological contexts.

Block 578. Referring to block 578, in some embodiments, the one or more cells are derived from a tissue of the test subject. Such cells may capture test subject-specific characteristics such as mutations, epigenetic features, or transcriptomic profiles, and may be used to validate findings observed in other model systems such as organoids or in silico predictions.

Block 580. Referring to block 580, in some embodiments, the one or more cells is derived from a cancerous tissue of the test subject. For instance, tumor cells may be used to evaluate responses to cytotoxic, targeted, or immunomodulatory agents, and may be particularly useful for longitudinal monitoring of therapeutic resistance or clonal evolution through successive rounds of treatment.

Block 581. Referring to block 581, in some embodiments, the one or more cells are from a tissue of a reference subject. The reference subject may be a healthy donor or an individual with a well-characterized clinical and molecular background. In some embodiments, the one or more cells are from a cohort of reference subjects, including individuals selected to reflect a range of demographic or genetic variables such as age, sex, ancestry, or known polymorphisms. The cells may be obtained from established biobanks, commercial cell repositories, or newly derived from donor tissue, including but not limited to skin, lung, gastrointestinal tract, kidney, liver, or other organ systems.

Reference-derived cells may serve multiple roles, such as providing a normative baseline for evaluating biological effects, controlling for assay variation, or enabling comparative modeling of disease versus non-disease states. For example, when evaluating the cytotoxic or molecular effects of a given therapy in a test subject's tumor-derived cell line, the inclusion of reference cells from healthy donors allows the identification of effects that are tumor-selective versus broadly cytotoxic. Moreover, data from reference cells may be used to calibrate high-throughput screens, normalize molecular readouts, or establish population-level thresholds for treatment response.

Block 582. Referring to block 582, in some embodiments, the one or more cells are from a cancerous tissue of the reference subject. These cancer-derived reference cells may represent diverse tumor types, molecular subtypes, or resistance phenotypes, and may be drawn from publicly available cancerous cells or from proprietary or newly established repositories. In some embodiments, such cells are from tumors carrying specific oncogenic alterations, such as KRAS, EGFR, BRAF, or TP53 mutations, or from cancers known to be resistant or sensitive to particular classes of therapies, including chemotherapy, small-molecule inhibitors, or immune checkpoint modulators.

By including cancer cells from reference subjects with known response profiles or defined molecular features, the system supports benchmarking of the test subject's therapeutic response in a broader biological and clinical context. For instance, the test subject's tumor-derived cells may be analyzed alongside reference tumor cells known to be responsive to a particular therapy. If the test subject's cells display similar response behavior, this may provide additional confidence in therapeutic selection. Conversely, if the test subject's cells deviate from known patterns, the divergence may point to unique features or emerging resistance mechanisms.

In some embodiments, a plurality of reference cancer cells are used, including 2, 3, 4, 5, or 10 or more distinct cell types, or ranging from 10 to 100, 20 to 200, or even more than 500 different cell types. In some such embodiments, each such cell is from a different subject in the cohort of reference subjects. In some such embodiments, more than 1 such cell type is from the same reference subject in the cohort of reference subjects.

These reference cancer cells may support population-scale analyses of treatment heterogeneity, help identify biomarker-treatment associations, or be used to train machine learning models to predict drug efficacy based on cell line profiles.

In all such embodiments, the inclusion of reference cells, both healthy and cancerous, provides experimental context, supports comparative modeling, and enhances the robustness and interpretability of results generated by the system for a given test subject.

Block 583. Referring to block 583, in some embodiments, the data from the system modeling human tissue additionally or alternatively comprises data collected after contacting each respective cell in a plurality of cells with a different respective therapy in a plurality of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Blocks 584-586. Referring to block 584-586, in some embodiments, the plurality of therapies comprises any of the therapies disclosed in block 530 and/or block 532 and/or block 534.

Block 587. Referring to block 587 information comprising the set of characteristics from the electronic medical record and the data from the system modeling human tissue are provided to an artificial intelligence (AI) component. Responsive to such input, output from the AI component is received specifying one or more care pathways for the medical condition.

Continuing to refer to block 587, in some embodiments specialized input engineering techniques, also referred to as prompt engineering, are used to optimize performance and accuracy of the AI component in generating care pathway recommendations.

For example, the set of characteristics may be structured into a standardized prompt format that includes labeled fields such as patient demographics, clinical history, laboratory values, molecular profiles, and current medications. In some embodiments the AI component is configured to operate on natural language input, and these structured data elements are converted into semantically meaningful sentences or paragraphs using domain-specific templates (e.g., “The patient is a 62-year-old male with stage III colorectal cancer, KRAS-mutated, with prior exposure to FOLFOX and currently progressing on therapy.”).

Additionally, in some embodiments, the input prompt includes contextual framing instructions that guide the LLM's response format, level of specificity, or scope. These instructions may include preambles such as: “Based on the following patient data and ex vivo drug response models, identify the most appropriate therapeutic strategy, including any clinical trial opportunities,” or “Summarize evidence-based care pathways for a patient with this profile and prioritize therapies by predicted efficacy.” In some embodiments, prompt suffixes or few-shot exemplars are appended to the input to demonstrate the desired output structure.

In some embodiments, the input prompt also includes metadata or experimental data from the system modeling human tissue, such as organoid or xenograft assay results, IC50 values, gene expression signatures, or resistance markers identified through in vitro screening. These elements may be provided as tabulated data or embedded as natural language summaries (e.g., “Organoid models from the test subject demonstrate sensitivity to anti-EGFR monoclonal antibodies and resistance to irinotecan.”).

In some embodiments, multi-turn prompt sequences are used to iteratively refine the output from the AI component, in which the initial AI component responses are re-evaluated using additional prompts that request justification, confidence ranking, or mechanistic rationale. In some embodiments, the AI component is primed with background clinical guidelines or knowledge bases (e.g., NCCN guidelines or published drug indications), either embedded in the prompt or retrieved dynamically via retrieval-augmented generation (RAG) methods.

Responsive to such input, output from the AI component is received specifying one or more care pathways for the medical condition. The output may include ranked treatment recommendations, citations to clinical evidence, predicted efficacy metrics, or annotations that explain the relationship between specific patient features and the suggested interventions. In certain embodiments, the output is further processed for downstream integration into clinical decision support systems, electronic health records, or care coordination platforms.

Block 588. Referring to block 588, in some embodiments, the medical condition comprises a cancer. The term “cancer” as used herein encompasses a broad array of malignant neoplastic disorders that may originate in virtually any tissue or organ system. In some embodiments, the cancer may be a carcinoma, including but not limited to non-small cell lung carcinoma, small cell lung carcinoma, colorectal carcinoma, pancreatic adenocarcinoma, gastric carcinoma, esophageal carcinoma, renal cell carcinoma, hepatocellular carcinoma, bladder carcinoma, cervical carcinoma, or prostate carcinoma.

In other embodiments, the cancer may involve hematologic malignancies, such as acute myeloid leukemia (AML), chronic lymphocytic leukemia (CLL), acute lymphoblastic leukemia (ALL), chronic myeloid leukemia (CML), multiple myeloma, or various subtypes of non-Hodgkin lymphoma and Hodgkin lymphoma.

Certain embodiments may also address gynecologic cancers, such as ovarian cancer, endometrial (uterine) cancer, and vulvar cancer. Other examples include breast cancer, including hormone receptor-positive, HER2-positive, and triple-negative subtypes.

Further embodiments may relate to neurological cancers, such as glioblastoma multiforme, astrocytoma, ependymoma, and medulloblastoma. Additionally, cutaneous malignancies, such as melanoma and squamous cell carcinoma of the skin, are contemplated.

In pediatric settings, embodiments may also pertain to childhood-specific cancers such as neuroblastoma, Wilms tumor (nephroblastoma), and retinoblastoma.

In some embodiments, the cancer may arise from a variety of tissues of origin, reflecting the diverse nature of oncogenesis across the human body. These tissues of origin may include, but are not limited to, the epithelial lining of the lung, the colonic mucosa, the pancreatic ducts, the gastric epithelium, the hepatic parenchyma, the renal cortex, the urinary bladder urothelium, the endometrial lining of the uterus, the ovarian surface epithelium, the prostatic acini, the mammary ducts, the skin epidermis, the central nervous system glial tissue, the lymphoid tissue of lymph nodes, the hematopoietic bone marrow, the thyroid follicular epithelium, the mesothelial lining of the pleura, the connective tissue of soft muscle and fat, the retinal neuroepithelium, and the testicular germinal epithelium. Each tissue type provides a distinct microenvironment and cellular architecture, influencing tumor behavior, treatment response, and clinical outcome.

Finally, embodiments may also encompass rare or less common malignancies such as mesothelioma, thyroid carcinoma, soft tissue sarcoma, Ewing sarcoma, and chondrosarcoma. Each of these cancer types may benefit from specialized diagnostic, prognostic, or therapeutic applications, including those contemplated in connection with block 588.

Block 589. Referring to block 589, in some embodiments, the medical condition comprises a cardiac condition, a pulmonary condition, a metabolic condition, an endocrine condition, an immune condition, an autoimmune condition, a rare disease, psychiatric disorder, or a neurological condition. Examples of cardiac conditions may include: Atrial Fibrillation (AFib), aortic stenosis, cardiac amyloidosis, arrhythmia, stroke. Examples of pulmonary conditions may include asthma, COPD, chronic bronchitis, pneumonia, Pulmonary fibrosis, tuberculosis, emphysema, Bronchiectasis, Bronchiolitis, Bronchitis, Lung cancer, Pneumothorax or atelectasis, and Pulmonary edema. Examples of metabolic or endocrine conditions may include: diabetes, high blood pressure, hypertensions, hemochromatosis, Hypertriglyceridemia, Phenylketonuria, Porphyria, Gaucher Disease, Fabry Disease, Mitochondrial disease, Lysosomal storage disease, Hypothyroidism, Cushing's Syndrome, Hashimoto Thyroiditis, Hypercalcemia, osteoporosis, Pituitary disorders, Congenital adrenal hyperplasia, PCOS, adrenal insufficiency, Acromegaly. Examples of immune or autoimmune conditions include: Crohn's disease, celiac disease, ulcerative colitis, Graves' disease, Hashimoto's thyroiditis, Addison's disease, Multiple sclerosis (MS), chronic inflammatory demyelinating polyneuropathy (CIDP), Guillain-Barre syndrome, Rheumatoid arthritis (RA), psoriatic arthritis, Sjögren's syndrome, Dermatomyositis, psoriasis, allergy. Examples of a neurological or mental health condition or psychiatric disorder include: epilepsy, autism, neuromuscular disorders, attention deficit disorder (ADD), Alzheimer's Disease, Amyotrophic Lateral Sclerosis (ALS), Ataxia, Bell's Palsy, Multiple Sclerosis, headaches or migraines, stroke, hydrocephalus, Encephalitis, Muscular Dystrophy, Parkinson's Disease, Treatment Resistant Depression, Major Depressive Disorder, Bipolar Disorder, and Schizophrenia.

Blocks 590-591. Referring to block 590, in some embodiments, the AI component is a large language model (LLM). Referring to block 591, in some embodiments the method comprises providing a set of natural language instructions to the large language model, were the set of natural language instructions provide a context to the LLM for identifying the one or more care pathways for the medical condition.

An LLM is a type of deep learning model based on the transformer architecture, pre-trained on vast amounts of textual data, typically ranging from hundreds of gigabytes to multiple terabytes in size. In some embodiments, the total training data approaches or exceeds a petabyte when counting all tokens processed over multiple training epochs. In some embodiments LLMs contain a very large number of parameters, typically 1 billion, 2 billion, 3 billion, 4 billion, 5 billion, 6 billion, 7 billion, 8 billion, 9 billion, 10 billion, 20 billion, 50 billion, 100 billion, 500 billion, 1 trillion, 2 trillion, 3 trillion, 4 trillion, 5 trillion, 6 trillion, 7 trillion, 8 trillion, 9 trillion, 10 trillion, 20 trillion or more parameters. In some embodiments LLMs contain between 1 billion and 40 trillion parameters. In some embodiments LLMs contain between 2 billion and 35 trillion parameters. In some embodiments LLMs contain between 3 billion and 30 trillion parameters.

In some embodiments LLMs contain between 4 billion and 25 trillion parameters. LLMs consist of deep stacks of transformer blocks, often numbering in the dozens or even hundreds. In some embodiments an LLM consists of between 10 and 800 transformer blocks. In some embodiments an LLM consists of between 20 and 750 transformer blocks. In some embodiments an LLM consists of between 30 and 600 transformer blocks. In some embodiments an LLM consists of between 40 and 550 transformer blocks. In some embodiments an LLM comprises 10, 20, 30, 40, 50, 60, 70, 80, 90, 100, 150, 200, 250, or 300 or more transformer blocks.

The example transformer architecture described in Vaswani et al., 2017, “Attention Is All You Need,” arXiv: 1706.03762 hereby incorporated by reference in its entirety, is an encoder-decoder model using self-attention to process sequences without recurrence. Transformer blocks make use of architectural components including multi-head self-attention, residual connections, and layer normalization. While some LLMs make use of some transformer blocks with both an encoder and decoder stack, many LLMs utilize transfer blocks with only the decoder (e.g., GPT models) or only the encoder (e.g., BERT models). For example, the Generative Pre-trained Transformer (GPT) family disclosed in Radford et al., 2018, “Improving Language Understanding by Generative Pre-Training,” OpenAI Blog, which is hereby incorporated by reference, and extended in later iterations including GPT-2 and GPT-3, employs a decoder-only architecture. Conversely, BERT, disclosed in Devlin et al., 2019, “BERT: Pre-training of Deep Bidirectional Transformers for Language Understanding,” arXiv: 1810.04805, which is hereby incorporated by reference, is an encoder-only model. Models like T5 disclosed in Raffel et al., 2020, “Exploring the Limits of Transfer Learning with a Unified Text-to-Text Transformer,” Journal of Machine Learning Research 21 (2020), pp. 1-67, hereby incorporated by reference, and BART disclosed in Lewis et al., 2020, “BART: Denoising Sequence-to-Sequence Pre-training for Natural Language Generation, Translation, and Comprehension,” arXiv: 1910.13461, hereby incorporated by reference, use a full encoder-decoder configuration.

LLMs are distinguished from smaller models by their scale. Whereas earlier transformer-based models contained tens of millions to hundreds of millions of parameters, LLMs typically have at least one billion, 10 billion, or 100 billion parameters. The scale of LLMs enables learning of sophisticated language representations, semantic generalizations, and long-range dependencies across diverse knowledge domains.

Smaller transformer models may include, for example, between 6 and 24 transformer blocks (or layers), depending on their architecture and use case. In contrast, an LLM often comprises 80 or more transformer layers, with some models having 96, 128, or even over 200 layers. For example, GPT-3 disclosed in Brown et al., 2020, “Language Models are Few-Shot Learners,” arXiv: 2005.14165, hereby incorporated by reference, includes 96 transformer layers and 175 billion parameters, while Google's PaLM disclosed in Chowdhery et al., 2022, “PaLM: Scaling Language Modeling with Pathways,” arXiv: 2204.02311, hereby incorporated by reference, includes 540 billion parameters across 118 layers.

LLMs also use wide embeddings (e.g., 2048, 4096, or more dimensions) and larger feed-forward and attention layers.

Whereas smaller transformer models may be trained on domain-specific datasets in the gigabyte range, LLMs are typically trained on massive, heterogeneous corpora containing hundreds of billions to trillions of tokens. These training corpora may include web text (e.g., Common Crawl), digitized books, Wikipedia, scientific articles, code repositories (e.g., GitHub), and other large-scale text resources. For instance, GPT-3 was trained on 300 billion tokens, and models like Gopher and Chinchilla disclosed in Hoffmann et al., 2022, “Training Compute-Optimal Large Language Models,” arXiv: 2203:15556, hereby incorporated by reference, similarly leverage multi-terabyte datasets. The size and diversity of training data contribute to the models' ability to generalize across multiple domains and tasks.

LLMs are distinguished from conventional machine learning models and earlier neural networks by several defining characteristics. First, LLMs employ deep transformer architectures, meaning they consist of a large number of sequentially stacked transformer blocks, often including 24, 48, 96, or more layers. In this context, “deep” refers to the vertical depth of the model architecture, which directly corresponds to the number of transformer layers through which data is processed. Second, LLMs contain extremely large numbers of trainable parameters, typically exceeding 1 billion parameters, and in some instances reaching or surpassing 100 billion, 500 billion, or even 1 trillion parameters. Third, LLMs typically use embedding dimensions of at least 768, 1024, or 2048 units, and in some implementations 4096, 8192, or more. Associated feed-forward network components often have intermediate layer sizes 2-4 times the embedding dimension, commonly in the range of 8,192 to 32,768 units or more. The multi-head attention mechanisms in these models typically use 12, 16, 32, or more attention heads, with each head operating over a defined projection subspace. Fourth, LLMs are trained on extremely large and diverse datasets, including corpora containing hundreds of billions to trillions of text tokens, representing total data volumes of hundreds of gigabytes to multiple terabytes. Fifth, by virtue of their architectural scale and data exposure, LLMs exhibit robust generalization capabilities across tasks and domains, and may demonstrate emergent abilities, such as few-shot learning, zero-shot inference, and compositional reasoning, without requiring task-specific retraining. These characteristics collectively differentiate LLMs from conventional machine learning models and shallower neural architectures.

Block 592. Referring to block 592, in some embodiments, the AI component is a denoising diffusion model or a variational autoencoder (VAE).

Diffusion models are a class of generative machine learning models that create output by simulating a gradual transformation from pure noise into structured content. The process begins by taking real data and adding small amounts of noise to it over many steps until it becomes indistinguishable from random noise, a procedure known as the forward diffusion process. The model is then trained to learn the reverse process: starting from noise, it progressively removes the noise step by step to reconstruct the original data. This reverse denoising is guided by a neural network, in some embodiments a U-Net architecture, which learns to predict either the original data or the noise added at each step. Diffusion models are highly controllable, enabling generation conditioned on text prompts, class labels, or other guidance.

A VAE is a type of generative model that learns to represent complex data, such as images or other high-dimensional inputs, in a compressed, probabilistic latent space. Unlike traditional autoencoders, which encode each input into a fixed vector, VAEs model the encoded representation as a probability distribution, typically a Gaussian, characterized by a learned mean and variance. This allows the model to generate new, realistic samples by sampling from the latent space rather than simply reconstructing known inputs. The architecture consists of two main components: an encoder that maps input data to a latent distribution, and a decoder that reconstructs the original data from samples drawn from this distribution. During training, the VAE minimizes two losses: a reconstruction loss, which measures how closely the output resembles the input, and a Kullback-Leibler (KL) divergence term, which regularizes the latent space by encouraging it to approximate a standard normal distribution.

VAEs are described in Dai et al., 2018, “Syntax-directed variational autoencoder for structured data,” arXiv: 1802.08786, Ghosh et al., “From variational to deterministic autoencoders,” arXiv: 1903.12436, 2019, Kusner et al., “Grammar variational autoencoder,” International Conference on Machine Learning, 2017; and Sønderby et al., “Ladder variational autoencoders,” Advances in Neural Information Processing Systems, 29, 2016, each of which is hereby incorporated by reference.

Block 593. Referring to block 593, in some embodiments, the method further comprises evaluating, for a respective care pathway in the one or more care pathways for the medical condition, an efficacy of the respective care pathway by exposing one or more test cells to a therapy associated with the respective care pathway and measuring an output from the one or more test cells.

Referring to block 593, in some embodiments, the method further comprises a biologically grounded evaluation step, in which each proposed care pathway is assessed for its potential efficacy using ex vivo or in vitro experimental models. For a given care pathway that includes a specific therapeutic regimen or sequence of interventions, the corresponding therapy (e.g., a small molecule drug, monoclonal antibody, chemotherapy agent, immunotherapy, or combination thereof) is applied to test cells (e.g., derived from the test subject or a representative model). These test cells may include, but are not limited to, patient-derived organoids, primary tumor cells, immortalized cell lines, or xenograft-derived cells, which recapitulate key biological features of the patient's condition.

Upon exposure to the therapy, one or more biological outputs are measured to evaluate therapeutic response. These outputs may include cell viability, apoptosis markers, proliferation rates, metabolic activity, or more advanced readouts such as single-cell transcriptomics, proteomic shifts, cytokine release, or epigenetic modifications. In some embodiments, dose-response curves are generated to derive quantitative measures such as IC₅₀ or EC₉₀ values, allowing direct comparison between alternative treatment strategies.

By integrating these biologically measured responses into the evaluation pipeline, the method provides a functional validation layer that complements the AI-predicted care pathways, ensuring that recommendations are not only statistically or algorithmically sound, but also demonstrate tangible efficacy in a patient-specific biological context. In certain embodiments, this evaluation may further inform a ranking or refinement of care pathways, enhancing the precision of personalized treatment planning.

Block 594. Referring to block 594, in some embodiments, the one or more test cells comprises a tissue organoid (see for instance blocks 520-534 on suitable organoids), a tissue culture, a xenograft tissue (see blocks 556-582 for suitable xenograph tissues), or one or more cells evaluated in one more microfluidics devices (see blocks 576-586 for such set ups).

Continuing to refer to block 594, in some embodiments, the one or more test cells used for evaluating therapeutic efficacy are selected from a range of biologically relevant models that replicate the tissue-specific and molecular characteristics of the medical condition. These may include tissue organoids, which are three-dimensional cell cultures derived from stem cells or primary tissues that self-organize to mimic the architecture and function of actual organs. Alternatively, the test cells may be in the form of 2D tissue cultures, which allow for rapid and scalable testing. In some embodiments, xenograft tissues, such as test subject derived xenografts (PDX) grown in immunocompromised mice, may be used to model in vivo drug responses. In other embodiments, the test cells may be housed in microfluidics-based devices or “organs-on-chips,” which enable dynamic perfusion and mechanical stimulation, simulating the physiological conditions of the human body and supporting high-resolution, real-time analysis of drug responses.

Block 595. Referring to block 595, in some embodiments, the test cells used in the evaluation process are test subject specific, meaning they are derived directly from the test subject. These may include biopsy-derived tumor cells, circulating tumor cells (CTCs), reprogrammed induced pluripotent stem cells (iPSCs), or normal somatic cells transformed for modeling purposes. This ensures that the biological assay reflects the unique genetic, epigenetic, and microenvironmental context of the individual patient, thereby enabling a highly personalized and relevant evaluation of therapeutic efficacy.

Block 596. Referring to block 596, in some embodiments, if the output measured from the one or more test cells satisfies predefined criteria, such as significant reduction in cell viability, induction of apoptosis, or biomarker expression consistent with therapeutic response, then the associated therapy is designated a matched therapy. This therapy is then identified as part of the respective care pathway tailored specifically for the test subject. The matching process confirms not only theoretical efficacy based on computational or clinical data, but also functional efficacy observed directly in patient-derived biological material.

Block 597. Referring to block 597, in some embodiments, when the measured output from the test cells fails to satisfy the target criteria, indicating insufficient therapeutic effect, toxicity, or resistance, the system dynamically adjusts the care pathway. This may involve identifying a modified therapy, such as a different drug, a combination regimen, a dose adjustment, or an alternative treatment modality altogether (e.g., shifting from chemotherapy to immunotherapy). This adaptive process ensures that the recommended care pathway is both evidence-informed and biologically validated, minimizing the risk of ineffective treatment.

Block 598. Referring to block 598, in some embodiments, once a respective care pathway has been selected, either as initially proposed or modified based on test cell response, it is reported to a healthcare provider responsible for managing the care of the test subject. This reporting may take the form of a structured digital summary integrated into the electronic health record (EHR), a clinical decision support alert, or a narrative report containing relevant patient data, assay results, therapeutic rationale, and supporting evidence. This empowers the clinician with actionable insights grounded in both computational prediction and biological validation.

Block 599. Referring to block 599, in some embodiments, the selected care pathway is administered as a therapy to the test subject, forming the final, clinical implementation step of the method in some embodiments. This may involve initiating the prescribed drug regimen, enrolling the test subject in a matched clinical trial, performing a surgical intervention, or applying any other medically appropriate action outlined in the care pathway. The end-to-end integration of AI-guided recommendation, optional functional testing, optional expert review, and clinical application represents a closed-loop precision medicine system, designed to improve outcomes by aligning treatment with the individual biology and clinical context of the test subject.

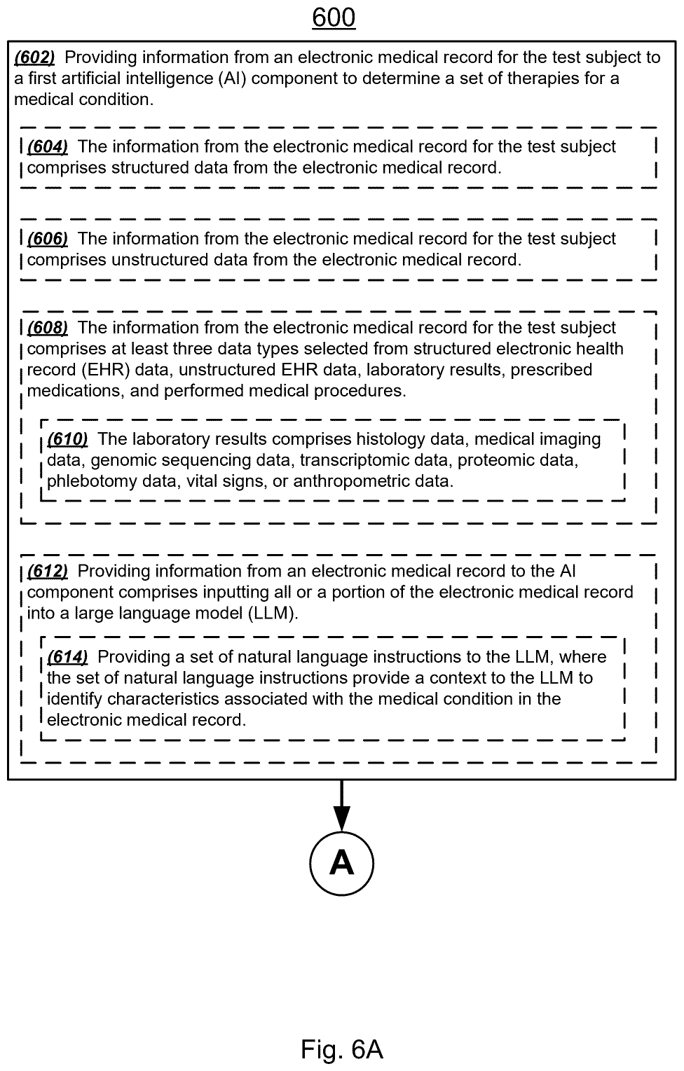

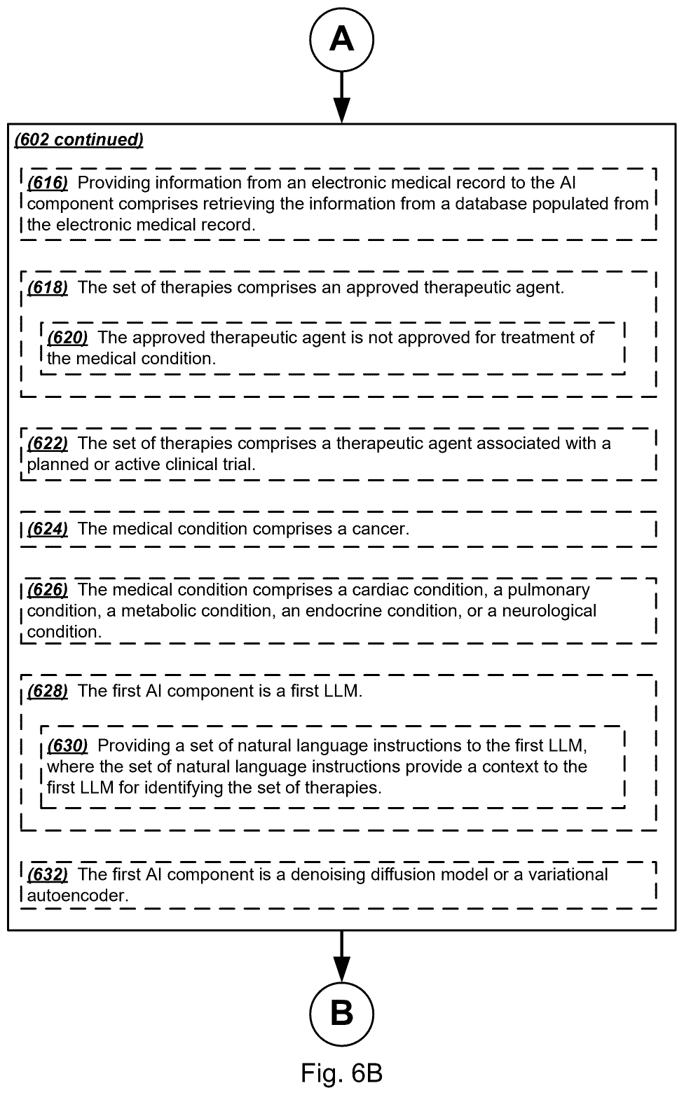

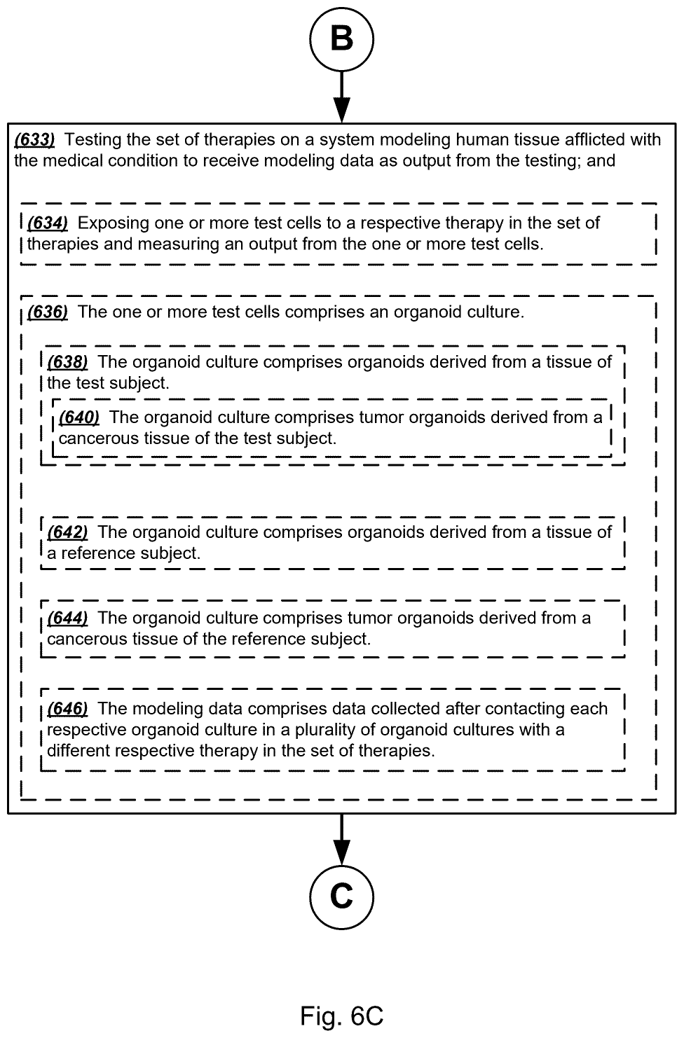

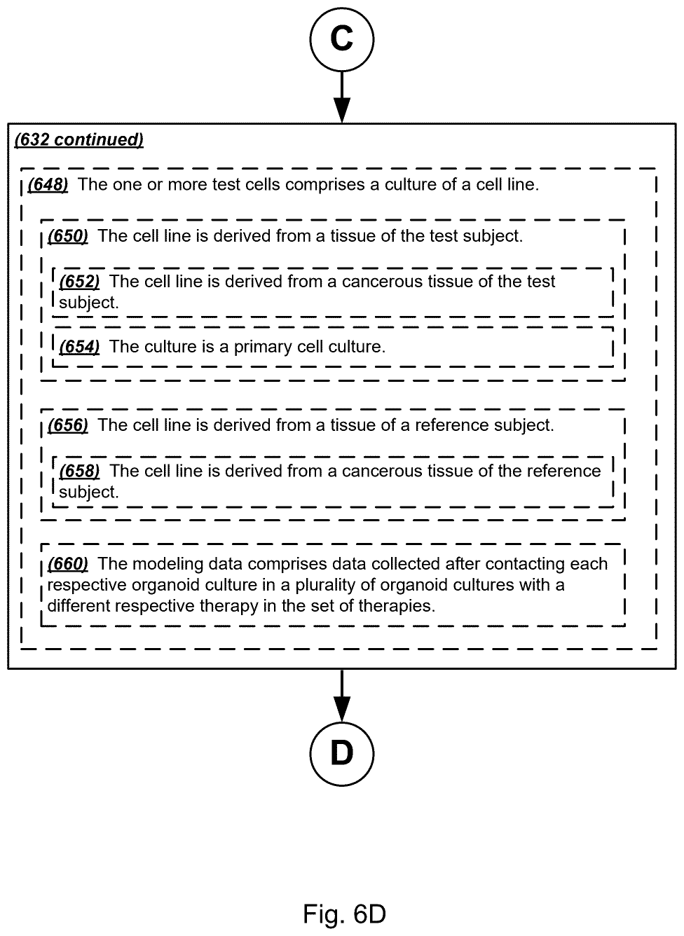

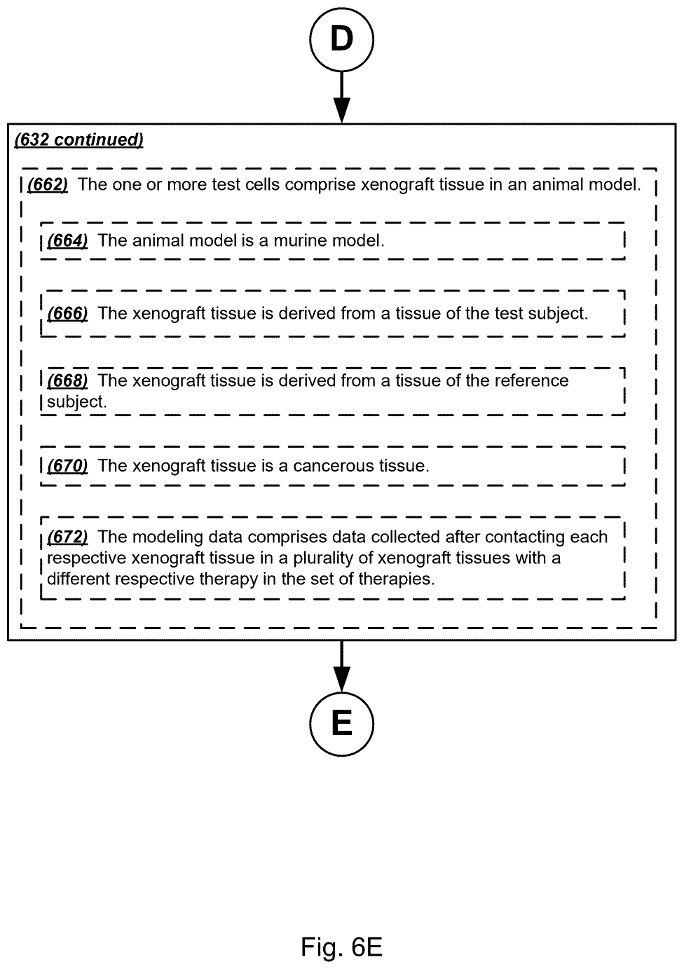

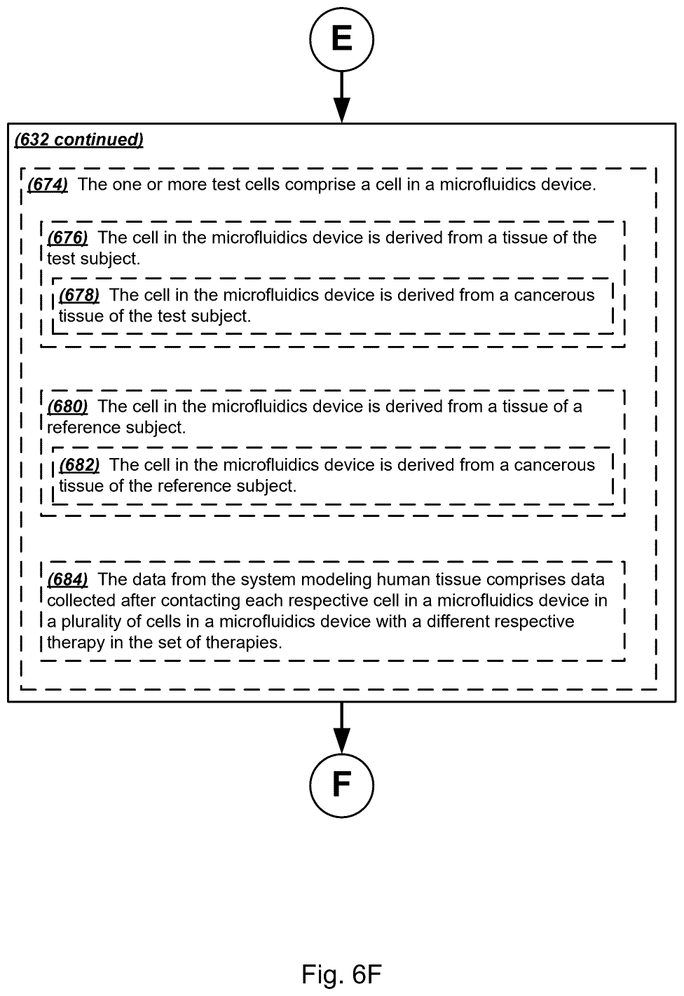

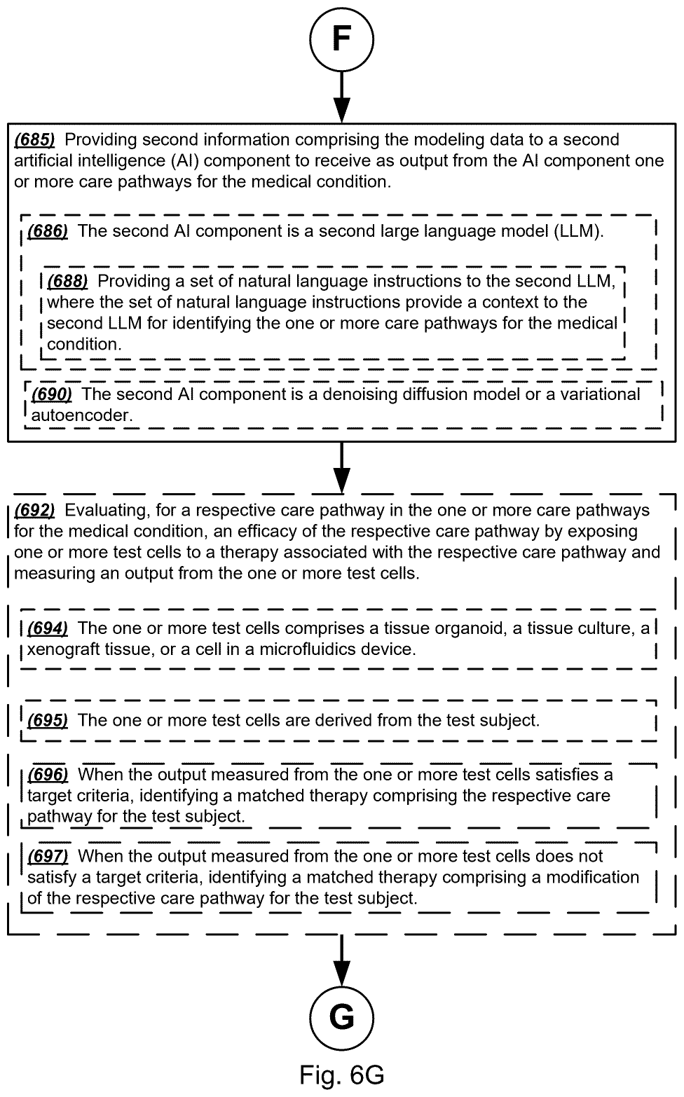

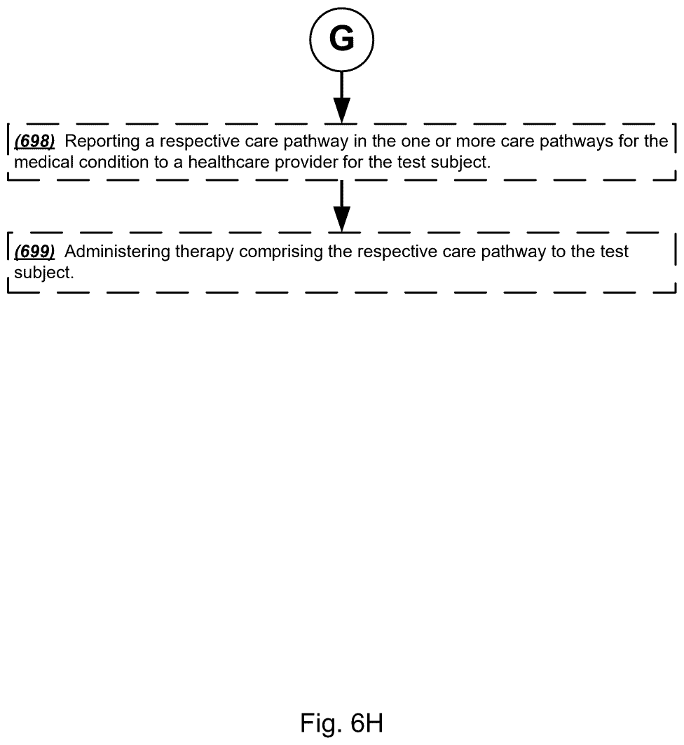

FIGS. 6A-6G collectively provide a flow chart of processes and features predicting care pathway options for a test subject, in accordance with some embodiments of the present disclosure.

The present disclosure provides a method 600 predicting care pathway options for a test subject. In some embodiments, all or a portion of method 600 is performed at a computer system having one or more processors and memory storing one or more programs for execution by the one or more processors.

Block 602. Referring to block 602, in some embodiments, information is provided from an electronic medical record for the test subject to a first artificial intelligence (AI) component to determine a set of therapies for a medical condition. As described in greater detail in block 501, electronic medical records, also referred to as electronic health records (EHRs), provide a useful resource for individualized health data, by combining large amounts of patient-specific data collected during the provision of healthcare services.

Block 604. Referring to block 604, in some embodiments, the information from the electronic medical record for the test subject comprises structured data from the electronic medical record. Nonlimiting examples of such structured data is described in block 504 above.

Block 606. Referring to block 606, in some embodiments, the information from the electronic medical record for the test subject comprises unstructured data from the electronic medical record. Nonlimiting examples of such structured data is described in block 506 above.

Block 608. Referring to block 608, in some embodiments, the information from the electronic medical record for the test subject comprises at least three data types selected from structured electronic health record (EHR) data, unstructured EHR data, laboratory results, prescribed medications, and performed medical procedures.

In some embodiments, the information retrieved from the electronic medical record (EMR) for the test subject comprises at least three distinct types of clinical data, selected from a group that includes: structured electronic health record (EHR) data, unstructured EHR data, laboratory results, prescribed medications, and performed medical procedures. Structured EHR data typically refers to discrete, codified entries stored in standardized fields, such as demographic details, diagnosis codes (e.g., ICD-10), procedure codes (e.g., CPT), vital signs, and other quantitative metrics that are easily searchable and interoperable. Unstructured EHR data, in contrast, consists of free-text narratives authored by clinicians, such as progress notes, consultation summaries, pathology and radiology reports, which often capture subtle clinical reasoning, symptom descriptions, contextual nuances, and temporal associations that are not available in structured formats.

Laboratory results encompass a wide range of diagnostic outputs, including blood counts, metabolic panels, molecular diagnostics, biomarker levels, and histopathology findings, all of which provide objective, measurable insights into the physiological and pathological status of the patient. Prescribed medications offer a window into the therapeutic history and pharmacologic management of the condition, indicating the classes of drugs used, dosing regimens, treatment durations, and potential drug interactions or side effects. Performed medical procedures provide information about diagnostic and therapeutic interventions, including surgical operations, biopsies, imaging studies (e.g., MRI, CT scans), and other interventional events that are critical to understanding the patient's clinical trajectory.

By requiring the inclusion of at least three data types, the system ensures a comprehensive, multi-dimensional representation of the patient's medical history and current status. This diversity of data inputs enables more accurate modeling, improved clinical inference, and richer context for downstream applications such as AI-based care pathway recommendations, therapy selection, risk stratification, or real-time clinical decision support.

Block 610. Referring to block 610, in some embodiments, the laboratory results comprises histology data, medical imaging data, genomic sequencing data, transcriptomic data, proteomic data, phlebotomy data, vital signs, or anthropometric data. Such data is described in further detail in block 510 above.

Blocks 612-614. Referring to block 612, in some embodiments, providing information from an electronic medical record to the AI component comprises inputting all or a portion of the electronic medical record into a large language model. Referring to block 614, in some embodiments, providing a set of natural language instructions to the large language model (LLM), where the set of natural language instructions provide a context to the LLM to identify characteristics associated with the medical condition in the electronic medical record. Examples of natural language instructions that can be used to input data in the LLM are described in blocks 512-514 above.

Block 616. Referring to block 616, in some embodiments, providing information from an electronic medical record to the AI component comprises retrieving the information from a database populated from the electronic medical record. For instance, in some embodiments, electronic medical records are processed on an on-going basis to identify relevant health information, which is then stored in a database, e.g., an indexable database, for the case of accessing the relevant information for analyses such as the methods described herein. Advantageously, curating relevant information from electronic health records ahead of time eliminates the need to process electronic medical records when performing the methods described herein, speeding up, by reducing the computational burden of, the processes described herein. More description of information from a database populated from the electronic medical record is described in block 516 above.

Block 618. Referring to block 618, in some embodiments, the set of therapies comprises an approved therapeutic agent. In some embodiments, the therapy has been approved for the medical condition being investigated. More description of such approval is described in block 530.

Block 620. Referring to block 620, in some embodiments, the approved therapeutic agent is not approved for treatment of the medical condition. In some embodiments, the therapy is used as an off-label therapy for the medical condition. In some embodiments, the therapy is not known as an off-label therapy for the medical condition. More description of use of such therapeutic agents in this capacity is described in block 532.

Block 622. Referring to block 622, in some embodiments, the set of therapies comprises a therapeutic agent associated with a planned or active clinical trial. In some embodiments, the clinical trial is still recruiting, such that a patient for which the therapy is identified may be enrolled in the clinical trial. More description of such clinical trials is provided in block 534.

Block 624. Referring to block 624, in some embodiments, the medical condition comprises a cancer. Nonlimiting examples of cancer in accordance with block 624 are described in block 588.

Block 526. Referring to block 626, in some embodiments, the medical condition comprises a cardiac condition, a pulmonary condition, a metabolic condition, an endocrine condition, an immune condition, an autoimmune condition, a rare disease, psychiatric disorder, or a neurological condition. Nonlimiting examples of such medical conditions is provide in block 589.

Blocks 628-630. Referring to block 628, in some embodiments, the first AI component is a first large language model (LLM). Referring to block 630, in some embodiments, the method comprises providing a set of natural language instructions to the first LLM, where the set of natural language instructions provide a context to the first LLM for identifying the set of therapies. More description of LLMs and the use of natural language instructions to input data into an LLM are described above in conjunction with blocks 590-591.

Block 632. Referring to block 632, in some embodiments, the first AI component is a denoising diffusion model or a variational autoencoder. Nonlimiting examples and descriptions of denoising diffusion model and variational autoencoder are provided in block 592.

Block 633. Referring to block 633, in some embodiments, the method further comprises a biological validation step, in which the set of therapies under consideration is tested using a system that models human tissue afflicted with the medical condition. This modeling system may take various forms, including tissue organoids, patient-derived cell cultures, three-dimensional bioprinted tissue constructs, xenograft models, and/or microfluidic organ-on-chip devices, all of which are engineered to replicate key structural and functional features of the affected human tissue. The purpose of this testing is to simulate how the disease-affected tissue would respond in a real biological context when exposed to specific therapeutic agents. By conducting this testing outside the body, yet in biologically faithful models, the method generates modeling data that reflects the pharmacodynamic and pharmacogenomic response of the tissue to each candidate therapy. This data may include measurements related to cellular viability, apoptosis induction, immune activation, metabolic shifts, or gene expression changes, and can be used to validate, rank, or refine the therapy options being considered.

Block 634. Referring to block 634, in some embodiments, the testing process comprises exposing one or more test cells, which may be derived from patient samples, disease-mimicking cell lines, or genetically modified cellular models, to a respective therapy from the set of therapies. The exposure is conducted under tightly controlled conditions, which may include defined dosages, timed intervals, and the presence of relevant microenvironmental factors such as immune cells or extracellular matrix components. Following this exposure, the system performs quantitative or qualitative measurements to determine the cellular response. Such output may include, but is not limited to, cell proliferation rates, cytotoxicity, expression of disease or response biomarkers (e.g., PD-L1, HER2), transcriptomic or proteomic changes, and resistance markers. In some implementations, high-content imaging or multi-omics technologies may be used to capture complex, multidimensional data. This experimental output serves as a functional, biologically grounded indicator of each therapy's efficacy or mechanism of action, and is integrated into the decision-making pipeline to inform selection of the most promising treatment for the test subject.

In some embodiments, the set of therapies includes any of the therapies, drug targets, and/or drug classes listed in block 528.

In some embodiments, the set of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 636. Referring to block 636, in some embodiments, the one or more test cells comprises an organoid culture. Referring to block 638, in some embodiments, the organoid culture comprises organoids derived from a tissue of the test subject. Organoid cultures, including their response to drug treatment, are described in blocks 520-522 above.

Block 640. Referring to block 640, in some embodiments, the organoid culture comprises tumor organoids derived from a cancerous tissue of the test subject. Organoid cultures comprising tumor organoids derived from a cancerous tissue of the test subject are described in blocks 520-522 above.

Blocks 642-644. Referring to block 642, in some embodiments, the organoid culture comprises organoids derived from a tissue of a reference subject. Referring to block 644, in some embodiments, the organoid culture comprises tumor organoids derived from a cancerous tissue of the reference subject. Derivation of such organoids is described in blocks 524-526 above.

Block 646. Referring to block 646, in some embodiments, the modeling data comprises data collected after contacting each respective organoid culture in a plurality of organoid cultures with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies. Example methods for screening potential therapies on tumor organoids are described in U.S. Patent Application Publication No. 2022/0392640, as well as U.S. Pat. Nos. 11,415,571 and 11,561,178, the disclosure of which are each incorporated herein by reference in their entireties. More descriptions of examples of such therapies and the number of such therapies is provided in blocks 528-532 above.

Block 648. Referring to block 648, in some embodiments, the one or more test cells comprises one or more cultures of one or more cell lines. Examples of such cultures of such cells lines is described in block 536.

Block 650. Referring to block 650, in some embodiments, the one or more cell lines is derived from a tissue of the test subject. Test subject-derived cell lines may capture test subject-specific characteristics such as mutations, epigenetic features, or transcriptomic profiles, and may be used to validate findings observed in other model systems such as organoids or in silico predictions.

Block 652. Referring to block 652, in some embodiments, one or more of the cell lines is derived from a cancerous tissue of the test subject. Tumor-derived cell lines may be used to evaluate responses to cytotoxic, targeted, or immunomodulatory agents, and may be particularly useful for longitudinal monitoring of therapeutic resistance or clonal evolution through successive rounds of treatment.

Block 654. Referring to block 654, in some embodiments, the culture is a primary cell culture. More details on primary cell cultures in accordance with some embodiments of block 654 is provided in block 542.

Block 656. Referring to block 656, in some embodiments, a cell line in the one or more cells lines is derived from a tissue of a reference subject. More disclosure of cell lines derived from a tissue of a reference subject is disclosed in block 544.

Block 658. Referring to block 658, in some embodiments, a cell line in the one or more cell lines is derived from a cancerous tissue of the reference subject. More disclosure of cell lines derived from a cancerous tissue of a reference subject is disclosed in block 546.

Block 660. Referring to block 660, in some embodiments, the modeling data comprises data collected after contacting each respective organoid culture in a plurality of organoid cultures with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 662. Referring to block 662, in some embodiments, the one or more test cells comprise xenograft tissue in an animal model. Example disclosure regarding such xenograph models is described in block 556.

Block 664. Referring to block 664, in some embodiments, the animal model is a murine model.

Block 666. Referring to block 666, in some embodiments, one or more of the xenograft models are established using tissue derived from the test subject. Subject-derived xenograft models may capture individual-specific characteristics such as somatic mutations, gene expression signatures, immune landscape, or epigenetic features. These personalized in vivo models may be used to validate results from other platforms, such as organoid systems or computational simulations, and may directly inform individualized therapeutic decision-making.

Block 668. Referring to block 668, in some embodiments, in some embodiments, the xenograft model is derived from a tissue of a reference subject. The reference subject may be a healthy donor or an individual with a well-characterized clinical and molecular profile. In some embodiments, the system includes xenograft models derived from a cohort of reference subjects selected to represent diversity across sex, age, ancestry, or known genetic polymorphisms. Tissues may be sourced from repositories, clinical samples, or newly acquired specimens and engrafted into appropriate immunocompromised or humanized animal hosts for in vivo study.

Reference-subject-derived xenograft models may serve multiple roles, such as establishing normative baselines for comparison, controlling for biological variability, or providing context for evaluating tumor-specific responses. For instance, when evaluating the therapeutic response of a test subject's tumor-derived xenograft, parallel evaluation of xenografts from healthy or non-diseased tissues may reveal tumor-specific vulnerabilities versus general systemic effects. Reference xenografts also aid in calibrating assay sensitivity and in interpreting treatment effects across different biological backgrounds.

Block 670. Referring to block 670, in some embodiments, the xenograft tissue is a cancerous tissue. These cancer-derived xenografts may represent diverse tumor types, molecular subtypes, or clinically relevant treatment profiles. In some embodiments, the tumors used for engraftment harbor defined genetic alterations such as TP53, EGFR, BRAF, KRAS, or ALK mutations, or are selected based on known responsiveness or resistance to classes of therapies including chemotherapy, targeted small molecules, antibody therapies, or immunotherapies.

Block 672. Referring to block 672, in some embodiments, the modeling data comprises data collected after contacting each respective xenograft tissue in a plurality of xenograft tissues with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 674. Referring to block 674, in some embodiments, the one or more test cells comprise one or more cells in a microfluidics device. In some embodiments, each such microfluidics device is a lab on a chip. For a review of lab on a chip technology see, for example, Lab Chip (23) (2023), the entire edition of which is dedicated to lab on a chip review articles, and of which the contents of the edition are incorporated herein by reference in its entirety. In some embodiments, individual cells are evaluated with the one or more microfluidics devices. In other embodiments, organoids, e.g., tumor organoids, are evaluated with the one or more microfluidic devices.

In some embodiments, the one or more cells may include 2, 3, 4, 5, 6, 7, 8, 9, 10, 11, 12, 13, 14, 15, 16, 17, 18, 19, or 20 or more cells. In certain embodiments, the number of cells ranges between 5 cells and 50 cells, 10 cells and 100 cells, 20 cells and 200 cells. In some embodiments, the number of cells is more than 100 cells, 1000 cells, 10,000 cells, 100,000 cells, or 1×10⁶ cells. These cells may differ by tissue of origin, genetic background, disease subtype, or known resistance profiles. The use of multiple cells enables broader screening for treatment responsiveness and allows for evaluation of how therapeutic effects vary across diverse biological contexts.

Block 676. Referring to block 676, in some embodiments, the cell in the microfluidics device is derived from a tissue of the test subject. Such cells may capture test subject-specific characteristics such as mutations, epigenetic features, or transcriptomic profiles, and may be used to validate findings observed in other model systems such as organoids or in silico predictions.

Block 678. Referring to block 678, in some embodiments, the one or more cells in the microfluidics device is derived from a cancerous tissue of the test subject. For instance, tumor cells may be used to evaluate responses to cytotoxic, targeted, or immunomodulatory agents, and may be particularly useful for longitudinal monitoring of therapeutic resistance or clonal evolution through successive rounds of treatment.

Block 680. Referring to block 680, in some embodiments, the one or more cells in the microfluidics device is derived from a tissue of a reference subject.

Referring to block 682, in some embodiments, the cell in the microfluidics device is derived from a cancerous tissue of the reference subject. The reference subject may be a healthy donor or an individual with a well-characterized clinical and molecular background. In some embodiments, the one or more cells are from a cohort of reference subjects, including individuals selected to reflect a range of demographic or genetic variables such as age, sex, ancestry, or known polymorphisms. The cells may be obtained from established biobanks, commercial cell repositories, or newly derived from donor tissue, including but not limited to skin, lung, gastrointestinal tract, kidney, liver, or other organ systems.

Reference-derived cells may serve multiple roles, such as providing a normative baseline for evaluating biological effects, controlling for assay variation, or enabling comparative modeling of disease versus non-disease states. For example, when evaluating the cytotoxic or molecular effects of a given therapy in a test subject's tumor-derived cell line, the inclusion of reference cells from healthy donors allows the identification of effects that are tumor-selective versus broadly cytotoxic. Moreover, data from reference cells may be used to calibrate high-throughput screens, normalize molecular readouts, or establish population-level thresholds for treatment response.

Block 684. Referring to block 684, in some embodiments, the data from the system modeling human tissue comprises data collected after contacting each respective cell in a microfluidics device in a plurality of cells in a microfluidics device with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 685. Referring to block 685, in some embodiments, the second information comprising the modeling data is provided to a second artificial intelligence (AI) component to receive as output from the AI component one or more care pathways for the medical condition.

Block 686. Referring to block 686, in some embodiments, the second AI component is a second large language model (LLM).

Block 688. Referring to block 688, in some embodiments, a set of natural language instructions is provided to the second LLM, where the set of natural language instructions provide a context to the second LLM for identifying the one or more care pathways for the medical condition.

Block 690. Referring to block 690, in some embodiments, the second AI component is a denoising diffusion model or a variational autoencoder. In some embodiments the denoising diffusion model or a variational autoencoder is as described in block 592.

Block 692. Referring to block 692, in some embodiments, the method also includes evaluating for a respective care pathway in the one or more care pathways for the medical condition, evaluating an efficacy of the respective care pathway by exposing one or more test cells to a therapy associated with the respective care pathway and measuring an output from the one or more test cells. More disclosure on evaluating for a respective care pathway in the one or more care pathways in accordance with block 692 is described in block 593.

Block 694. Referring to block 694, in some embodiments, the one or more test cells comprises a tissue organoid (see for instance blocks 520-534 on suitable organoids), a tissue culture, a xenograft tissue see blocks 556-582 for suitable xenograph tissues), or one or more cells evaluated in one or more microfluidics devices (see blocks 576-586 for such set ups).

Continuing to refer to block 694, in some embodiments, the one or more test cells used for evaluating therapeutic efficacy are selected from a range of biologically relevant models that replicate the tissue-specific and molecular characteristics of the medical condition. These may include tissue organoids, which are three-dimensional cell cultures derived from stem cells or primary tissues that self-organize to mimic the architecture and function of actual organs. Alternatively, the test cells may be in the form of 2D tissue cultures, which allow for rapid and scalable testing. In some embodiments, xenograft tissues, such as test subject derived xenografts (PDX) grown in immunocompromised mice, may be used to model in vivo drug responses. In other embodiments, the test cells may be housed in microfluidics-based devices or “organs-on-chips,” which enable dynamic perfusion and mechanical stimulation, simulating the physiological conditions of the human body and supporting high-resolution, real-time analysis of drug responses.

Block 695. Referring to block 695, in some embodiments, the one or more test cells are derived from the test subject. In some embodiments, the test cells used in the evaluation process in accordance with block 695 are test subject specific, meaning they are derived directly from the test subject. These may include biopsy-derived tumor cells, circulating tumor cells (CTCs), reprogrammed induced pluripotent stem cells (iPSCs), or normal somatic cells transformed for modeling purposes. This ensures that the biological assay reflects the unique genetic, epigenetic, and microenvironmental context of the individual patient, thereby enabling a highly personalized and relevant evaluation of therapeutic efficacy.

Block 696. Referring to block 696, in some embodiments, when the output measured from the one or more test cells meets or exceeds predefined criteria (e.g. a target criteria), such as significant reduction in cell viability, induction of apoptosis, or biomarker expression consistent with therapeutic response, then the associated therapy is designated a matched therapy. This therapy is then identified as part of the respective care pathway tailored specifically for the test subject. The matching process confirms not only theoretical efficacy based on computational or clinical data, but also functional efficacy observed directly in patient-derived biological material.

Block 697. Referring to block 697, in some embodiments, when the measured output from the test cells fails to satisfy the target criteria, indicating insufficient therapeutic effect, toxicity, or resistance, the system dynamically adjusts the care pathway. This may involve identifying a modified therapy, such as a different drug, a combination regimen, a dose adjustment, or an alternative treatment modality altogether (e.g., shifting from chemotherapy to immunotherapy). This adaptive process ensures that the recommended care pathway is both evidence-informed and biologically validated, minimizing the risk of ineffective treatment.

Block 698. Referring to block 698, in some embodiments, the method also includes reporting a respective care pathway in the one or more care pathways for the medical condition to a healthcare provider for the test subject. This reporting may take the form of a structured digital summary integrated into the electronic health record (EHR), a clinical decision support alert, or a narrative report containing relevant patient data, assay results, therapeutic rationale, and supporting evidence. This empowers the clinician with actionable insights grounded in both computational prediction and biological validation.

Block 699. Referring to block 699, in some embodiments, the method also includes administering therapy comprising the respective care pathway to the test subject. This may involve initiating the prescribed drug regimen, enrolling the test subject in a matched clinical trial, performing a surgical intervention, or applying any other medically appropriate action outlined in the care pathway. The end-to-end integration of AI-guided recommendation, optional functional testing, optional expert review, and clinical application represents a closed-loop precision medicine system, designed to improve outcomes by aligning treatment with the individual biology and clinical context of the test subject.

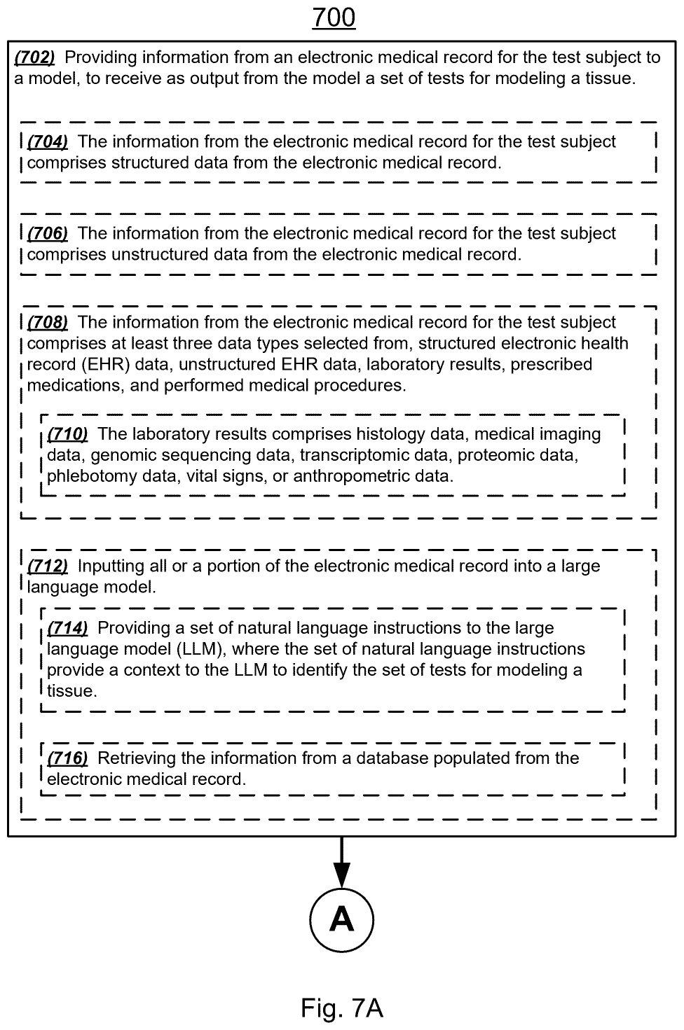

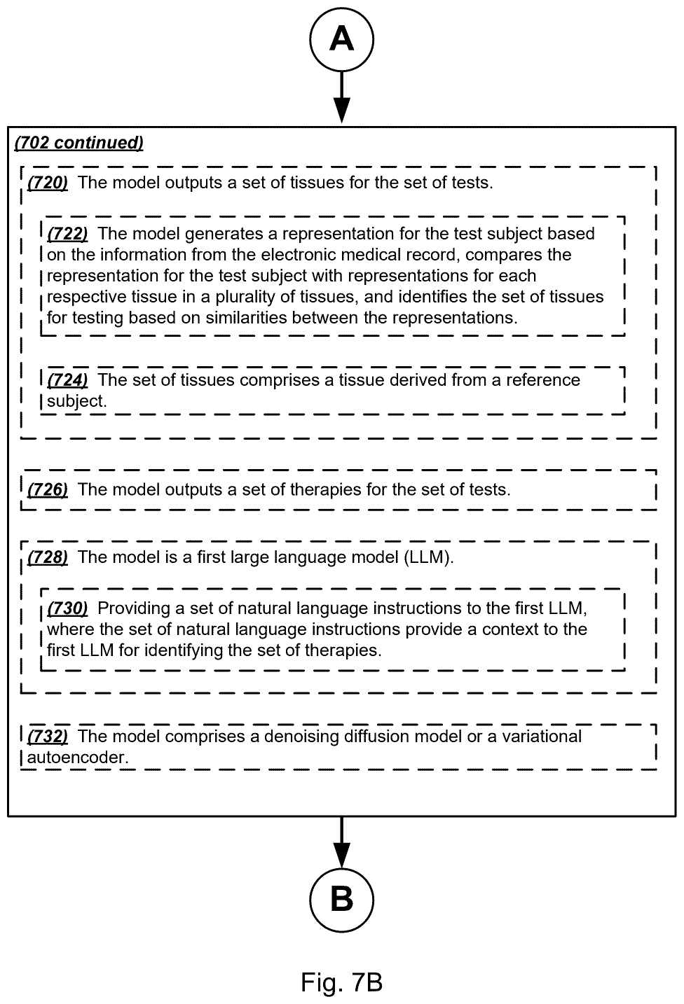

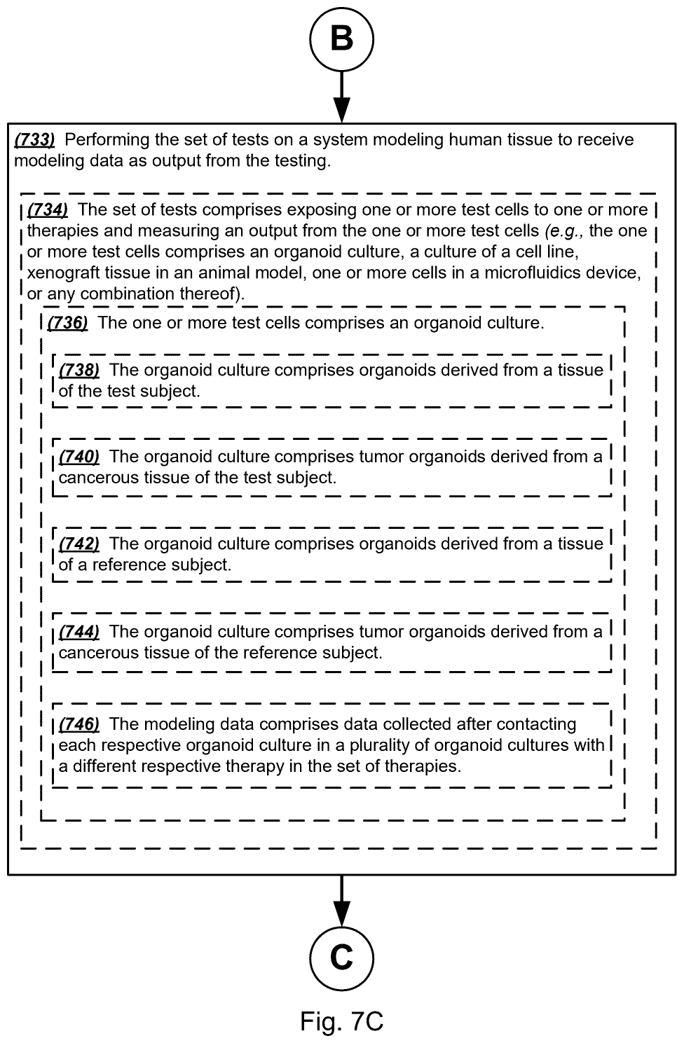

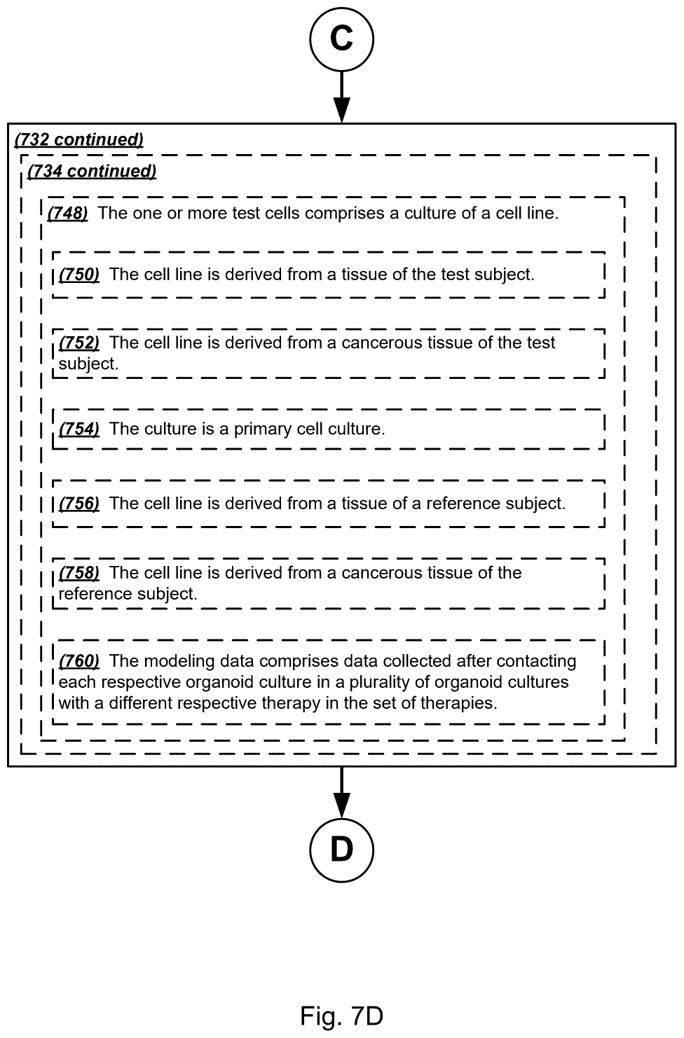

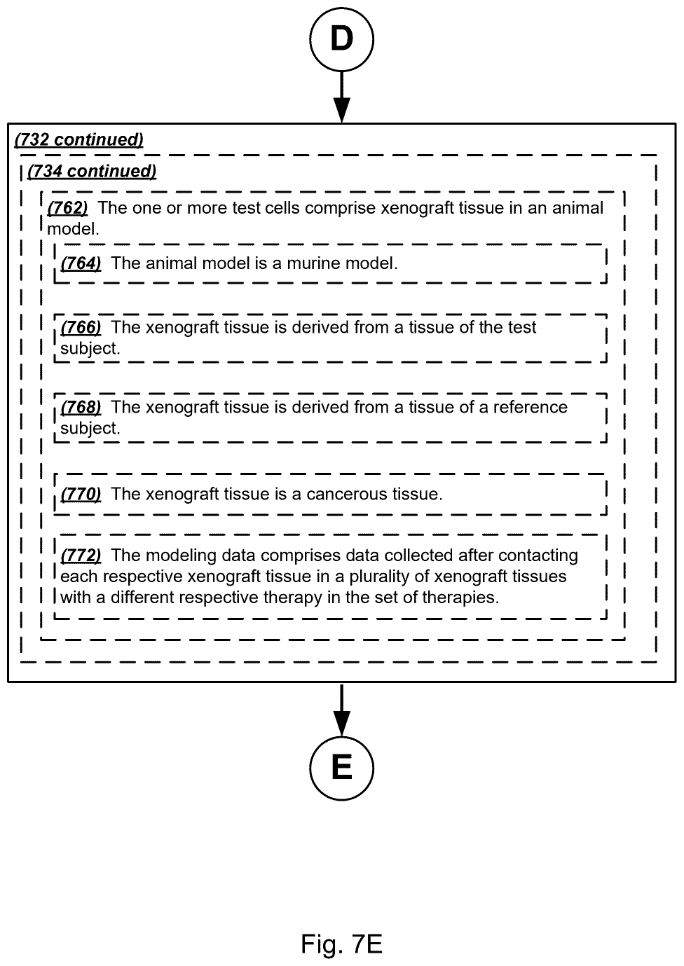

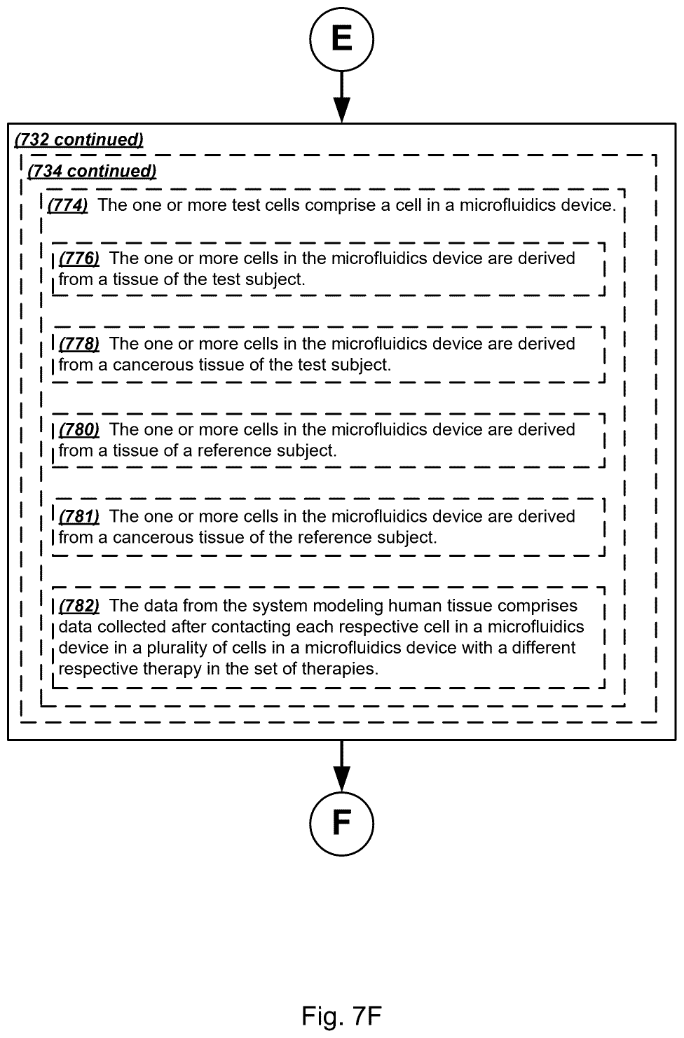

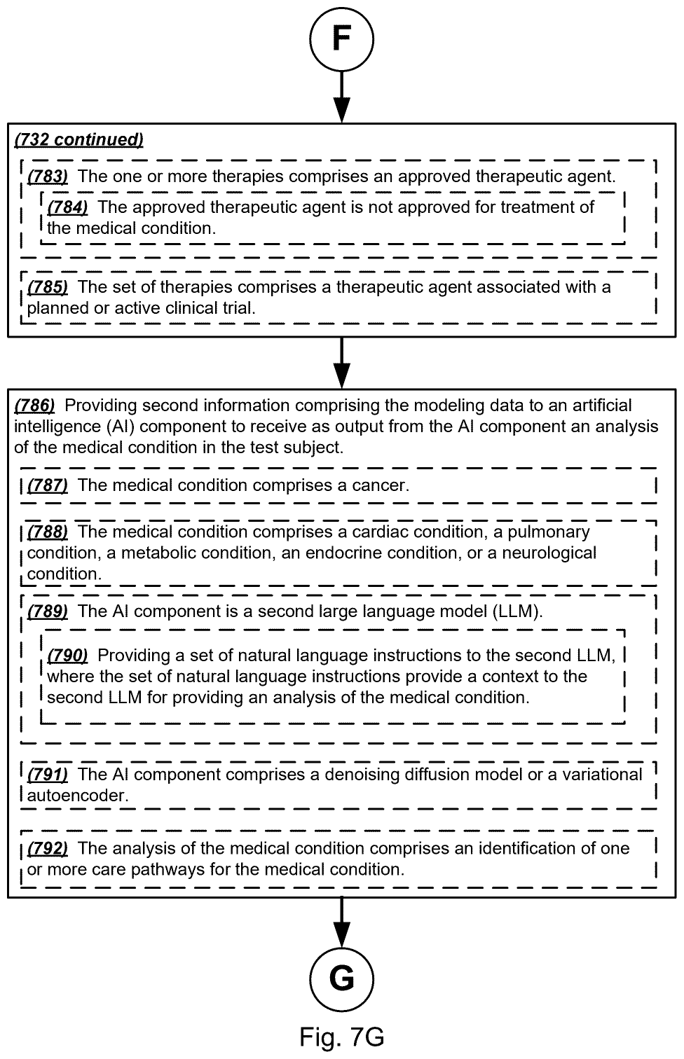

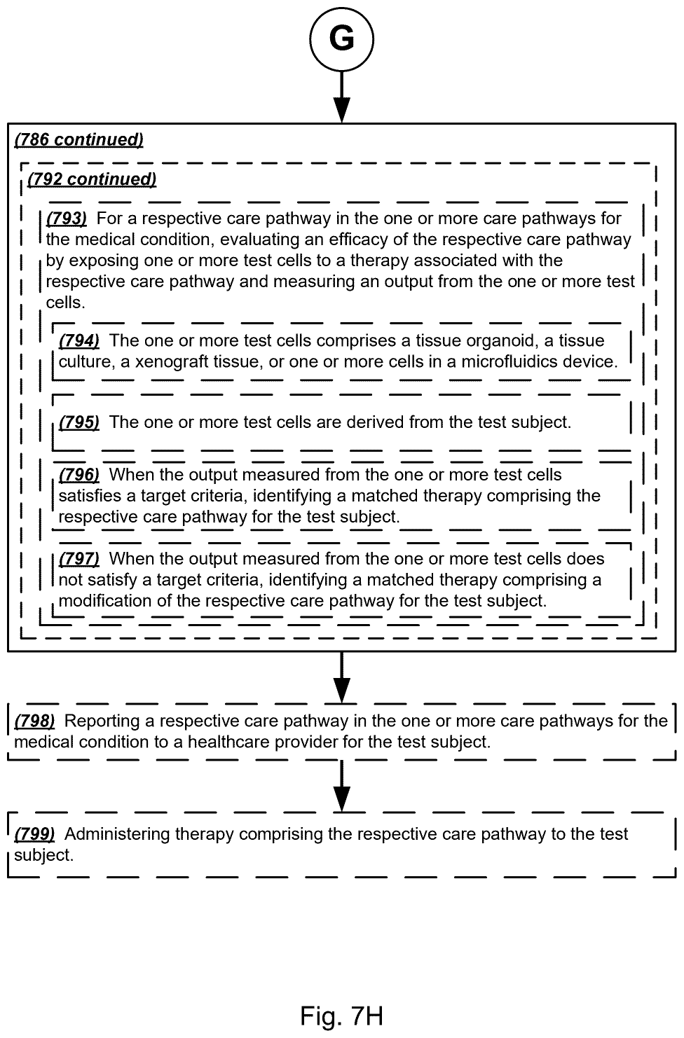

FIGS. 7A-7G collectively provide a flow chart of processes and features for evaluating a medical condition in a test subject, in accordance with some embodiments of the present disclosure.

The present disclosure provides a method 700 for evaluating a medical condition in a test subject. In some embodiments, all or a portion of method 700 is performed at a computer system having one or more processors and memory storing one or more programs for execution by the one or more processors.

Block 702. Referring to block 702, in some embodiments, information from an electronic medical record for the test subject is provided to a model, to receive as output from the model a set of tests for modeling a tissue. As described in greater detail above, electronic medical records, also referred to as electronic health records (EHRs), provide a useful resource for individualized health data, by combining huge amounts of patient-specific data collected during the provision of healthcare services. Examples of electronic medical records are provided in block 504 above.

Block 704. Referring to block 704, in some embodiments, the information from the electronic medical record for the test subject comprises structured data from the electronic medical record. Examples of structured data from the electronic medical record are described in block 504 above.

Block 706. Referring to block 706, in some embodiments, the information from the electronic medical record for the test subject comprises unstructured data from the electronic medical record. Examples of unstructured data from the electronic medical record are described in block 506 above.

Block 708. Referring to block 708, in some embodiments, the information from the electronic medical record for the test subject comprises at least three data types selected from, structured electronic health record (EHR) data, unstructured EHR data, laboratory results, prescribed medications, and performed medical procedures.

In some embodiments, retrieving the set of characteristics from the electronic medical record (EMR) comprises evaluating at least three distinct data types to construct a comprehensive patient profile that informs subsequent clinical analysis or decision-making processes. These data types may be selected from a group that includes: structured EHR data, such as diagnosis codes (e.g., ICD-10), vital signs, problem lists, and coded clinical observations; unstructured EHR data, such as free-text clinical notes, imaging reports, and narrative summaries, which often contain nuanced clinical insights not captured in structured formats; laboratory results, including standard blood panels, molecular diagnostics, biomarkers, and pathology reports, which provide objective, quantitative data on the patient's biological state; prescribed medications, which indicate ongoing and past therapeutic interventions, dosage regimens, and potential drug-drug interactions; and performed medical procedures, such as surgeries, diagnostic imaging, biopsies, or interventional treatments, which reflect both the severity and history of the medical condition.

In some embodiments, sophisticated data parsing and integration tools, such as natural language processing (NLP), clinical ontologies, and interoperability standards (e.g., FHIR or HL7), are employed to extract, normalize, and reconcile these data sources into a unified and interpretable format. By requiring the inclusion of at least three of these modalities, the method ensures a multifaceted and context-rich understanding of the patient's health status, treatment history, and disease progression. This integrated dataset can then be used as input for downstream processes such as AI-driven care pathway generation, eligibility assessment for clinical trials, or functional therapy testing, thereby enhancing the personalization and accuracy of medical decision support.

Block 710. Referring to block 710, in some embodiments, the laboratory results comprises histology data, medical imaging data, genomic sequencing data, transcriptomic data, proteomic data, phlebotomy data, vital signs, or anthropometric data. Further examples of laboratory results are provided in block 510 above.

Blocks 712-714. Referring to block 712, in some embodiments, the method includes inputting all or a portion of the electronic medical record into a large language model (LLM). Referring to block 714, in some embodiments, a set of natural language instructions is provided to the LLM, where the set of natural language instructions provide a context to the LLM to receive the set of tests for modeling a tissue.

Continuing to refer to block 712, in some embodiments specialized input engineering techniques, also referred to as prompt engineering, are used to optimize performance and accuracy of the model in generating a set of tests for modeling a tissue.

For example, the information from the electronic medical record for the test subject may be structured into a standardized prompt format that includes labeled fields such as patient demographics, clinical history, laboratory values, molecular profiles, and current medications. In some embodiments the model is configured to operate on natural language input, and these structured data elements are converted into semantically meaningful sentences or paragraphs using domain-specific templates (e.g., “The patient is a 62-year-old male with stage III colorectal cancer, KRAS-mutated, with prior exposure to FOLFOX and currently progressing on therapy.”).

Additionally, in some embodiments, the input prompt includes contextual framing instructions that guide the model's response format, level of specificity, or scope. These instructions may include preambles such as: “Based on the following patient data, identify the most appropriate set of tests for modeling a tissue.” In some embodiments, prompt suffixes or few-shot exemplars are appended to the input to demonstrate the desired output structure.

In some embodiments, multi-turn prompt sequences are used to iteratively refine the output from the model, in which the initial model responses are re-evaluated using additional prompts that request justification, confidence ranking, or mechanistic rationale. In some embodiments, the model is primed with background clinical guidelines or knowledge bases (e.g., NCCN guidelines or published drug indications), either embedded in the prompt or retrieved dynamically via retrieval-augmented generation (RAG) methods.

Responsive to such input, output from the model is received specifying one or more set of tests for modeling a tissue.

In some embodiments the model is any machine learning model, such as those described under in the section entitled “Classifier,” above.

Block 716. Referring to block 716, in some embodiments, information is retrieved from a database populated from the electronic medical record. For instance, in some embodiments, electronic medical records are processed on an on-going basis to identify relevant health information, which is then stored in a database, e.g., an indexable database, for the case of accessing the relevant information for analyses such as the methods described herein. Advantageously, curating relevant information from electronic health records ahead of time eliminates the need to process electronic medical records when performing the methods described herein, speeding up, by reducing the computational burden of, the processes described herein. Further description of such databases is provided in block 516, above.

Block 720. Referring to block 720, in some embodiments, the model outputs a set of tissues for the set of tests. In some embodiments the set of tests is 1, 2, 3, 4, 5, 6, 7, 8, 9, 10, 20 or 30 or more tests. In some embodiments the set of tissues is 1, 2, 3, 4, 5, 6, 7, 8, 9, 10, 20 or 30 or more tissues.

Block 722. Referring to block 722, in some embodiments, the model generates a representation for the test subject based on the information from the electronic medical record, compares the representation for the test subject with representations for each respective tissue in a plurality of tissues, and identifies the set of tissues for testing based on similarities between the representations. In some embodiments, the model identifies a set of tissues, e.g., reference tissues available from a tissue collection, that best match the biology of the patient. An example method for identifying tumor organoids that closely match the biology of a human tissue sample using an mRNA transcriptional profile is described in U.S. Patent Application Publication No. US2024/0355485, the content of which is incorporated herein by reference in its entirety. Other examples for identifying matching tissues are described in U.S. Patent Publication No. 2022/0059240, the disclosure of which is incorporated herein by reference in its entirety.

Block 724. Referring to block 724, in some embodiments, the set of tissues comprises a tissue derived from a reference subject.

Block 726. Referring to block 726, in some embodiments, the model outputs a set of therapies for the set of tests. In some embodiments, the model identifies a plurality of drugs that are predicted to have favorable therapeutic effects on the subject based on the patient's biology, as ascertained from the electronic health record of the patent.

Block 728. Referring to block 728, in some embodiments, the model is a first large language model (LLM). Further description of LLMs is provided in block 590.

Block 730. Referring to block 730, in some embodiments, a set of natural language instructions is provided to the first LLM, where the set of natural language instructions provide a context to the first LLM for identifying the set of therapies. Further description of providing context to an LLM using natural language processing is provided in blocks 587 and 591 above.

Block 732. Referring to block 732, in some embodiments, the model comprises a denoising diffusion model or a variational autoencoder. Example denoising diffusion models and variational autoencoders are described in block 529 above.

Block 733. Referring to block 733, in some embodiments, the set of tests is performed on a system modeling human tissue to receive modeling data as output from the testing.

Block 734. Referring to block 734, in some embodiments, the set of tests comprises exposing one or more test cells to one or more therapies and measuring an output from the one or more test cells (e.g., the one or more test cells comprises an organoid culture, a culture of a cell line, xenograft tissue in an animal model, one or more cells in a microfluidics device, or any combination thereof.

In some embodiments, the one or more therapies includes any of the therapies, drug targets, and/or drug classes listed in block 528.

In some embodiments, the one or more therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 736. Referring to block 736, in some embodiments, the one or more test cells comprises an organoid culture. An example method for culturing tumor organoids is described in U.S. Pat. No. 11,629,385, the content of which is incorporated herein by reference in its entirety. In some embodiments, an organoid, e.g., a tumor organoid, is co-cultured with one or more immune or effector cells. In some embodiments, the one or more immune or effector cells are derived from the same patient as is the organoid (e.g., the tumor organoid). Example methods for co-culturing organoids and immune or effector cells are described in U.S. Patent Application Publication No. 2023/0036156, the content of which is incorporated herein by reference in its entirety.

Block 738-740. Referring to block 738, in some embodiments, the organoid culture comprises organoids derived from a tissue of the test subject. Referring to block 740, in some embodiments, the organoid culture comprises tumor organoids derived from a cancerous tissue of the test subject. In some embodiments, the test subject-derived organoids are co-cultured with immune or effector cells from the same patient, in order to better model the tumor microenvironment.

In some such embodiments, these organoids are generated specifically to evaluate oncological conditions, such as solid tumors or metastatic lesions. The organoids may be established from biopsy or surgical resection specimens obtained from the test subject's tumor, enabling ex vivo modeling of the subject's specific cancer phenotype. These tumor-derived organoids preserve key molecular and histological characteristics of the original malignancy, including genomic mutations, epigenetic profiles, cellular heterogeneity, and microenvironmental interactions.

In some embodiments, the organoids may be derived from a variety of tissue types, either healthy or cancerous (tumor), including but not limited to colorectal, pancreatic, breast, lung, liver, kidney, ovarian, prostate, gastric, or brain tissues. The organoids may also represent subtypes within cancers from such origins (e.g., triple-negative breast cancer, KRAS-mutant colorectal cancer, EGFR-mutant non-small cell lung cancer), and may retain the tumor's sensitivity or resistance to specific classes of therapies. In some embodiments these organoids are cultured in 3D matrices such as extracellular matrix hydrogels under conditions that support their growth and differentiation, including the use of tumor-specific media enriched with growth factors, signaling modulators, or niche-supporting co-factors.

In various embodiments, a plurality of organoids, ranging from one to thousands, are cultured in a high-throughput or semi-automated format, such as in multiwell plates or microfluidic arrays. These organoids may then be exposed to one or more classes of anti-cancer agents, including cytotoxic chemotherapies (e.g., platinum compounds, taxanes), targeted therapies (e.g., tyrosine kinase inhibitors, monoclonal antibodies), hormone therapies, immunotherapies (e.g., checkpoint inhibitors), or investigational agents under preclinical evaluation. In some embodiments, drug testing includes both single-agent and combinatorial regimens, with variable concentrations and exposure durations to simulate clinically relevant dosing schedules.

Organoid response to drug treatment may be assessed using a range of readouts, including but not limited to (i) cell viability assays (e.g., ATP-based luminescence, live/dead staining), apoptosis or proliferation markers (e.g., cleaved caspase-3, Ki-67), transcriptomic changes (e.g., via RNA sequencing or qPCR), morphological alterations captured through high-content imaging, resistance signatures or pathway activation profiles (e.g., phospho-protein analysis, immunofluorescence).

In some embodiments, organoids are used to determine the susceptibility or resistance of the test subject's tumor to a given therapeutic class, based on empirically observed response patterns. The resulting data may indicate drug efficacy, partial resistance, or complete refractoriness, and may guide the selection of personalized treatment strategies, including avoidance of ineffective agents or identification of alternative, more responsive regimens.

Block 742. Referring to block 742, in some embodiments, the organoid culture comprises organoids derived from a tissue of a reference subject.

In some embodiments, the organoids are derived not from a single reference subject but from a cohort of reference subjects, which may include healthy individuals selected to represent a normative biological baseline. In some embodiments the cohort of reference subjects is 2, 3, 4, 5, 6, 7, 8, 9, 10, 11, 12, 13, 14, 15, 16, 17, 18, 19, 20 or more different reference subjects. In some embodiments the cohort of reference subjects is 20, 30, 40, 50, 60, 70, 80, 90, 100, 110, 120, 130, 140, 150, 160, 170, 180, 190, or 200 different reference subjects. In some embodiments such reference subjects are healthy. In some embodiments the cohort subjects represent a broad array of different tissues, such as 5 or more, 10 or more, 15 or more, or 20 or more different tissues, meaning that for each such respective tissue, there is at least 1, 2, 3, 4, or 5 or more organoids derived from the respective tissue.

The tissue used to generate the organoids may be obtained from elective biopsies, surgical discards, or donor programs, and may include tissue from organs such as the colon, lung, liver, pancreas, kidney, prostate, or breast.

Organoids derived from healthy reference subjects enable systematic comparison between diseased and non-diseased tissues under identical experimental conditions. For example, when evaluating drug effects, such reference organoids provide a control group for determining whether observed cytotoxicity is tumor-specific or reflects general tissue toxicity. They may also help identify off-target effects, reveal differential pathway activation in healthy versus cancerous tissues, or serve as controls for molecular profiling assays such as RNA sequencing or proteomics. Moreover, organoids from a healthy cohort can be stratified by age, sex, ancestry, or other biological factors to assess population-level variability in baseline tissue response.

Block 744. Referring to block 744, in some embodiments, the organoid culture comprises tumor organoids derived from a cancerous tissue of the reference subject. Organoids derived from a cancerous tissue of a reference subject are further described in block 526.

Block 746. Referring to block 746, in some embodiments, the modeling data comprises data collected after contacting each respective organoid culture in a plurality of organoid cultures with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies. Further description of such therapies is disclosed in block 528 above.

Block 748. Referring to block 748, in some embodiments, the one or more test cells comprises a culture of a cell line. Cell line models provide a robust, reproducible, and experimentally tractable platform for studying human biological responses in vitro. These models may be used to evaluate cellular proliferation, signaling pathway activity, drug sensitivity, metabolic responses, gene expression, or molecular pathway perturbations.

In some embodiments, a plurality of distinct cell lines are used. For example, the plurality of cell lines may include 2, 3, 4, 5, 6, 7, 8, 9, 10, 11, 12, 13, 14, 15, 16, 17, 18, 19, or 20 or more cell lines. In certain embodiments, the number of cell lines ranges between 5 cell lines and 50 cell lines, 10 cell lines and 100 cell lines, 20 cell lines and 200 cell lines. These cell lines may differ by tissue of origin, genetic background, disease subtype, or known resistance profiles. The use of multiple cell lines enables broader screening for treatment responsiveness and allows for evaluation of how therapeutic effects vary across diverse biological contexts.

Block 750. Referring to block 750, in some embodiments, the cell line is derived from a tissue of the test subject. Test subject-derived cell lines may capture test subject-specific characteristics such as mutations, epigenetic features, or transcriptomic profiles, and may be used to validate findings observed in other model systems such as organoids or in silico predictions.

Block 752. Referring to block 752, in some embodiments, the cell line is derived from a cancerous tissue of the test subject. Tumor-derived cell lines may be used to evaluate responses to cytotoxic, targeted, or immunomodulatory agents, and may be particularly useful for longitudinal monitoring of therapeutic resistance or clonal evolution through successive rounds of treatment.

Block 754. Referring to block 754, in some embodiments, the culture is a primary cell culture. These may be established directly from freshly resected or biopsied tissue without immortalization and maintained for a limited duration to preserve in vivo-like behavior. In some embodiments, combinations of primary and immortalized cell lines may be used in parallel to compare stable, scalable in vitro models with short-term, high-fidelity representations of patient biology.

In embodiments using multiple cell lines, data may be collected under standardized or perturbed conditions, with or without therapeutic agents, and subjected to comparative analysis across the full set of lines. This approach supports both individualized therapeutic modeling for the test subject and broader cohort-level or population-level inferences about treatment efficacy, drug resistance, or molecular mechanism of action.

Block 756. Referring to block 756, in some embodiments, the cell line is derived from a tissue of a reference subject. The reference subject may be a healthy donor or an individual with a well-characterized clinical and molecular background. In some embodiments, the cell lines are derived from a cohort of reference subjects, including individuals selected to reflect a range of demographic or genetic variables such as age, sex, ancestry, or known polymorphisms. The cell lines may be obtained from established biobanks, commercial cell repositories, or newly derived from donor tissue, including but not limited to skin, lung, gastrointestinal tract, kidney, liver, or other organ systems.

Reference-derived cell lines may serve multiple roles, such as providing a normative baseline for evaluating biological effects, controlling for assay variation, or enabling comparative modeling of disease versus non-disease states. For example, when evaluating the cytotoxic or molecular effects of a given therapy in a test subject's tumor-derived cell line, the inclusion of reference cell lines from healthy donors allows the identification of effects that are tumor-selective versus broadly cytotoxic. Moreover, data from reference cell lines may be used to calibrate high-throughput screens, normalize molecular readouts, or establish population-level thresholds for treatment response.

Block 758. Referring to block 758, in some embodiments, the cell line is derived from a cancerous tissue of the reference subject. Further description of providing a cell line derived from a cancerous tissue of the reference subject is provided in block 546 above.

Block 760. Referring to block 760, in some embodiments, the modeling data comprises data collected after contacting each respective organoid culture in a plurality of organoid cultures with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 762. Referring to block 762, in some embodiments, the one or more test cells comprise xenograft tissue in an animal model. Xenograft models provide a robust, reproducible, and experimentally tractable in vivo platform for studying human biological responses. These models may be used to evaluate tumor growth kinetics, metastatic potential, signaling pathway activity, therapeutic response, pharmacodynamics, immune interactions, or other molecular and cellular perturbations under physiologic conditions.

In some embodiments, a plurality of distinct xenograft models is used. For example, the plurality of xenograft models may include 2, 3, 4, 5, 6, 7, 8, 9, 10, 11, 12, 13, 14, 15, 16, 17, 18, 19, or 20 or more xenograft models. In certain embodiments, the number of xenograft models ranges between 5 and 50, 10 and 100, 20 and 200, or more. These xenograft models may differ by tissue of origin, genetic background, disease subtype, tumor microenvironment features, or known treatment resistance profiles. The use of multiple xenograft models enables broader evaluation of therapeutic effects and allows for studying response variability across diverse biological and clinical contexts.

Block 764. Referring to block 764, in some embodiments, the animal model is a murine model.

Block 766. Referring to block 766, in some embodiments, the xenograft tissue is derived from a tissue of the test subject. Subject-derived xenograft models may capture individual-specific characteristics such as somatic mutations, gene expression signatures, immune landscape, or epigenetic features. These personalized in vivo models may be used to validate results from other platforms, such as organoid systems or computational simulations, and may directly inform individualized therapeutic decision-making.

Referring to block 768, in some embodiments, the xenograft tissue is derived from a tissue of a reference subject. The reference subject may be a healthy donor or an individual with a well-characterized clinical and molecular profile. In some embodiments, the system includes xenograft models derived from a cohort of reference subjects selected to represent diversity across sex, age, ancestry, or known genetic polymorphisms. Tissues may be sourced from repositories, clinical samples, or newly acquired specimens and engrafted into appropriate immunocompromised or humanized animal hosts for in vivo study.

Reference-subject-derived xenograft models may serve multiple roles, such as establishing normative baselines for comparison, controlling for biological variability, or providing context for evaluating tumor-specific responses. For instance, when evaluating the therapeutic response of a test subject's tumor-derived xenograft, parallel evaluation of xenografts from healthy or non-diseased tissues may reveal tumor-specific vulnerabilities versus general systemic effects. Reference xenografts also aid in calibrating assay sensitivity and in interpreting treatment effects across different biological backgrounds.

Referring to block 770, in some embodiments, the xenograft tissue is a cancerous tissue (e.g., of a reference subject or the test subject).

Block 772. Referring to block 772, in some embodiments, the modeling data comprises data collected after contacting each respective xenograft tissue in a plurality of xenograft tissues with a different respective therapy in the set of therapies. Cancer-derived reference xenografts may represent diverse tumor types, molecular subtypes, or clinically relevant treatment profiles. In some embodiments, the tumors used for engraftment harbor defined genetic alterations such as TP53, EGFR, BRAF, KRAS, or ALK mutations, or are selected based on known responsiveness or resistance to classes of therapies including chemotherapy, targeted small molecules, antibody therapies, or immunotherapies.

Block 774. Referring to block 774, in some embodiments, the one or more test cells comprises one or more cells (e.g., examined) in a microfluidics device. Nonlimiting examples of suitable microfluidics device are described in block 576. In some embodiments, the one or more cells is a single cell. In some embodiments, the one or more cells is an organoid, e.g., a tumor organoid.

Block 776. Referring to block 776, in some embodiments, the one or more cells (e.g., examined) in the microfluidics device is derived from a tissue of the test subject. Such cells may capture test subject-specific characteristics such as mutations, epigenetic features, or transcriptomic profiles, and may be used to validate findings observed in other model systems such as organoids or in silico predictions.

Block 778. Referring to block 778, in some embodiments, the one or more cells (e.g., examined) in the microfluidics device is derived from a cancerous tissue of the test subject. For instance, tumor cells may be used to evaluate responses to cytotoxic, targeted, or immunomodulatory agents, and may be particularly useful for longitudinal monitoring of therapeutic resistance or clonal evolution through successive rounds of treatment.

Block 780. Referring to block 780, in some embodiments, the one or more cells (e.g., examined) in the microfluidics device is derived from a tissue of a reference subject. Such cells may capture test subject-specific characteristics such as mutations, epigenetic features, or transcriptomic profiles, and may be used to validate findings observed in other model systems such as organoids or in silico predictions.

Block 781. Referring to block 781, in some embodiments, the cell in the microfluidics device is derived from a cancerous tissue of the reference subject. For instance, tumor cells may be used to evaluate responses to cytotoxic, targeted, or immunomodulatory agents, and may be particularly useful for longitudinal monitoring of therapeutic resistance or clonal evolution through successive rounds of treatment.

Referring to block 782, in some embodiments, the data from the system modeling human tissue comprises data collected after contacting each respective cell in a microfluidics device in a plurality of cells in a microfluidics device with a different respective therapy in the set of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 783. Referring to block 783, in some embodiments, the one or more therapies comprises an approved therapeutic agent (e.g., approved for use in humans).

In some embodiments, the approved therapeutic agent is specifically approved for use in treating a medical condition being investigated, such as a particular form of cancer, inflammatory disease, neurological disorder, infectious disease, or metabolic condition. Approval, in this context, refers to formal authorization issued by a national or supranational regulatory authority, such as the U.S. Food and Drug Administration (FDA), the European Medicines Agency (EMA), the Pharmaceuticals and Medical Devices Agency (PMDA) in Japan, Health Canada, the Therapeutic Goods Administration (TGA) in Australia, or other analogous bodies, that permits the marketing and clinical use of the therapeutic agent for one or more specified indications.

Such approval is typically granted following a rigorous evaluation of preclinical data, safety studies, and human clinical trial results demonstrating efficacy, safety, and quality. The approved indications for a given therapeutic agent are usually documented in a product's official labeling or Summary of Product Characteristics (SmPC), which outlines the disease(s) or condition(s) for which the agent is authorized, recommended dosages, contraindications, and administration routes. In the context of the present method, a therapeutic agent that is approved for the specific condition under investigation may serve as a reference or benchmark against which the responses of the test subject's organoids are compared.

Block 784. Referring to block 784, in some embodiments, the approved therapeutic agent is not approved for treatment of the medical condition.

In these cases, the therapy may still be used under what is commonly referred to as off-label use, where a licensed medical product is administered for a disease or condition not explicitly covered by its regulatory approval. Off-label usage may arise when clinical experience, smaller-scale studies, or real-world data support the potential effectiveness of the therapy for a new indication that has not yet undergone formal regulatory review for that specific use.

Off-label use is common in areas of unmet clinical need, such as oncology, rare diseases, or pediatric medicine, where approved therapies may be limited or non-existent. While physicians are generally permitted to prescribe medications off-label based on their clinical judgment, such uses are not typically promoted by manufacturers and may not be reimbursed by insurers unless supported by strong clinical evidence or practice guidelines. In some embodiments, the present method evaluates how test subject-derived organoids respond to such off-label therapies, either confirming or challenging anecdotal evidence or clinical assumptions about the efficacy of these agents in a new disease context.

In further embodiments, the therapeutic agent is neither approved for the condition nor widely recognized as a candidate for off-label use. In this case, the therapy may be considered investigational or experimental, meaning it has not been subject to regulatory evaluation for any formal indication or may still be in preclinical or early-phase clinical trials. The inclusion of such unapproved or unrecognized therapies in the set of tested agents allows the method to identify novel treatment candidates, reposition existing drugs, or generate data to support future clinical investigation. In oncology, for instance, the system may be used to evaluate kinase inhibitors, immunomodulatory agents, or biologics that are approved for one tumor type but show promising activity in another, mechanistically related context.

In all such embodiments, whether the therapy is approved for the condition, approved for other conditions, used off-label, or entirely unapproved, the organoid-based modeling framework described herein allows for empirical evaluation of therapeutic efficacy on a personalized, biologically relevant platform. This facilitates treatment prioritization, risk-benefit analysis, and identification of unexpected vulnerabilities in a test subject's tumor or tissue model, enabling precision medicine.

Block 785. Referring to block 785, in some embodiments, the set of therapies comprises a therapeutic agent associated with a planned or active clinical trial. Examples of such clinical trials and therapeutic agents is provided in block 534 above.

Block 786. Referring to block 786, in some embodiments, the second information comprising the modeling data is provided to an artificial intelligence (AI) component to receive as output from the AI component an analysis of the medical condition in the test subject.

Block 787. Referring to block 787, in some embodiments, the medical condition comprises a cancer. Nonlimiting examples of cancer are provided in block 588.

Block 788. Referring to block 788, in some embodiments, the medical condition comprises a cardiac condition, a pulmonary condition, a metabolic condition, an endocrine condition, an immune condition, an autoimmune condition, a rare disease, psychiatric disorder, or a neurological condition. Examples of cardiac conditions may include: Atrial Fibrillation (AFib), aortic stenosis, cardiac amyloidosis, arrhythmia, stroke. Examples of pulmonary conditions may include asthma, COPD, chronic bronchitis, pneumonia, Pulmonary fibrosis, tuberculosis, emphysema, Bronchiectasis, Bronchiolitis, Bronchitis, Lung cancer, Pneumothorax or atelectasis, and Pulmonary edema. Examples of metabolic or endocrine conditions may include: diabetes, high blood pressure, hypertensions, hemochromatosis, Hypertriglyceridemia, Phenylketonuria, Porphyria, Gaucher Disease, Fabry Disease, Mitochondrial disease, Lysosomal storage disease, Hypothyroidism, Cushing's Syndrome, Hashimoto Thyroiditis, Hypercalcemia, osteoporosis, Pituitary disorders, Congenital adrenal hyperplasia, PCOS, adrenal insufficiency, Acromegaly. Examples of immune or autoimmune conditions include: Crohn's disease, celiac disease, ulcerative colitis, Graves' disease, Hashimoto's thyroiditis, Addison's disease, Multiple sclerosis (MS), chronic inflammatory demyelinating polyneuropathy (CIDP), Guillain-Barre syndrome, Rheumatoid arthritis (RA), psoriatic arthritis, Sjögren's syndrome, Dermatomyositis, psoriasis, allergy. Examples of a neurological or mental health condition or psychiatric disorder include: epilepsy, autism, neuromuscular disorders, attention deficit disorder (ADD), Alzheimer's Disease, Amyotrophic Lateral Sclerosis (ALS), Ataxia, Bell's Palsy, Multiple Sclerosis, headaches or migraines, stroke, hydrocephalus, Encephalitis, Muscular Dystrophy, Parkinson's Disease, Treatment Resistant Depression, Major Depressive Disorder, Bipolar Disorder, Schizophrenia.

Block 789. Referring to block 789, in some embodiments, the AI component is a second LLM.

Block 790. Referring to block 790, in some embodiments, a set of natural language instructions is provided to the second LLM, where the set of natural language instructions provides a context to the second LLM for providing an analysis of the medical condition.

Block 791. Referring to block 791, in some embodiments, the AI component comprises a denoising diffusion model or a variational autoencoder. Further description of denoising diffusion model and variational autoencoders is provided in block 592.

Block 792. Referring to block 792, in some embodiments, the analysis of the medical condition comprises an identification of one or more care pathways for the medical condition.

Block 793. Referring to block 793, in some embodiments, for a respective care pathway in the one or more care pathways for the medical condition, an efficacy of the respective care pathway is evaluated by exposing one or more test cells to a therapy associated with the respective care pathway and measuring an output from the one or more test cells.

Continuing to refer to block 783, in some embodiments, the method comprises a biologically grounded evaluation step, in which each proposed care pathway is assessed for its potential efficacy using ex vivo or in vitro experimental models. For a given care pathway that includes a specific therapeutic regimen or sequence of interventions, the corresponding therapy (e.g., a small molecule drug, monoclonal antibody, chemotherapy agent, immunotherapy, or combination thereof) is applied to test cells (e.g., derived from the test subject or a representative model). These test cells may include, but are not limited to, patient-derived organoids, primary tumor cells, immortalized cell lines, or xenograft-derived cells, which recapitulate key biological features of the patient's condition.

Upon exposure to the therapy, one or more biological outputs are measured to evaluate therapeutic response. These outputs may include cell viability, apoptosis markers, proliferation rates, metabolic activity, or more advanced readouts such as single-cell transcriptomics, proteomic shifts, cytokine release, or epigenetic modifications. In some embodiments, dose-response curves are generated to derive quantitative measures such as IC₅₀ or EC₉₀ values, allowing direct comparison between alternative treatment strategies.

By integrating these biologically measured responses into the evaluation pipeline, the method provides a functional validation layer that complements the AI-predicted care pathways, ensuring that recommendations are not only statistically or algorithmically sound, but also demonstrate tangible efficacy in a patient-specific biological context. In certain embodiments, this evaluation may further inform a ranking or refinement of care pathways, enhancing the precision of personalized treatment planning.

Block 794. Referring to block 794, in some embodiments, the one or more test cells comprises a tissue organoid (see, for example, for instance blocks 520-534 on suitable organoids), a tissue culture, a xenograft tissue (see, for example, blocks 556-582 for suitable xenograph tissues), or one or more cells (e.g., examined) in a microfluidics device (see, for example, blocks 576-586 for such set ups).

Block 795. Referring to block 795, in some embodiments, the one or more test cells are derived from the test subject. These may include biopsy-derived tumor cells, circulating tumor cells (CTCs), reprogrammed induced pluripotent stem cells (iPSCs), or normal somatic cells transformed for modeling purposes. This ensures that the biological assay reflects the unique genetic, epigenetic, and microenvironmental context of the test subject, thereby enabling a highly personalized and relevant evaluation of therapeutic efficacy.

Block 795. Referring to block 796, in some embodiments, if the output measured from the one or more test cells meets or exceeds predefined criteria, such as significant reduction in cell viability, induction of apoptosis, or biomarker expression consistent with therapeutic response, then the associated therapy is designated a matched therapy. This therapy is then identified as part of the respective care pathway tailored specifically for the test subject. The matching process confirms not only theoretical efficacy based on computational or clinical data, but also functional efficacy observed directly in patient-derived biological material.

Block 797. Referring to block 797, in some embodiments, when the measured output from the test cells fails to satisfy the target criteria, indicating insufficient therapeutic effect, toxicity, or resistance, the system dynamically adjusts the care pathway. This may involve identifying a modified therapy, such as a different drug, a combination regimen, a dose adjustment, or an alternative treatment modality altogether (e.g., shifting from chemotherapy to immunotherapy). This adaptive process ensures that the recommended care pathway is both evidence-informed and biologically validated, minimizing the risk of ineffective treatment.

Block 798. Referring to block 798, in some embodiments, the method also includes reporting a respective care pathway in the one or more care pathways for the medical condition to a healthcare provider for the test subject. This reporting may take the form of a structured digital summary integrated into the electronic health record (EHR), a clinical decision support alert, or a narrative report containing relevant patient data, assay results, therapeutic rationale, and supporting evidence. This empowers the clinician with actionable insights grounded in both computational prediction and biological validation.

Block 799. Referring to block 799, in some embodiments, the method also includes administering therapy comprising the respective care pathway to the test subject. This may involve initiating a prescribed drug regimen, enrolling the test subject in a matched clinical trial, performing a surgical intervention, or applying any other medically appropriate action outlined in the care pathway.

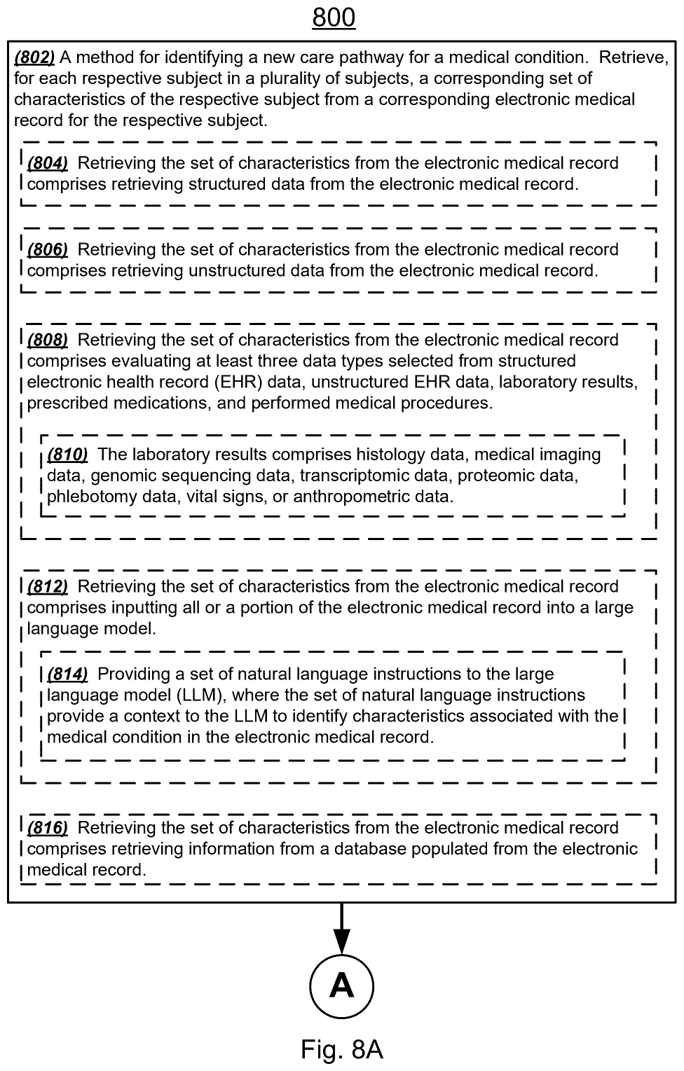

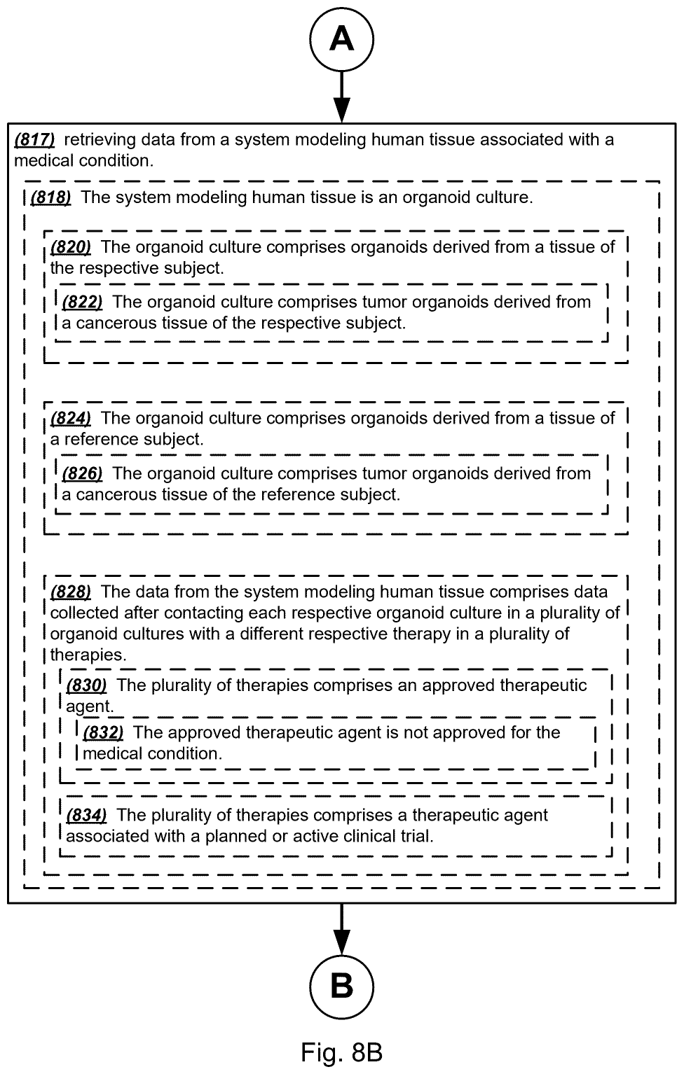

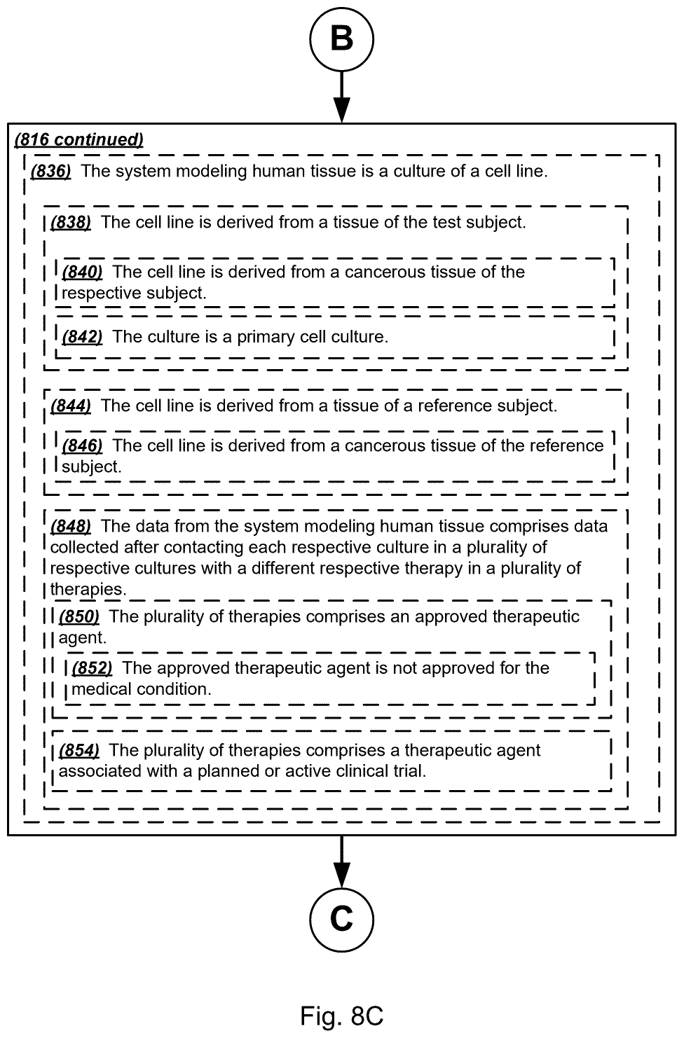

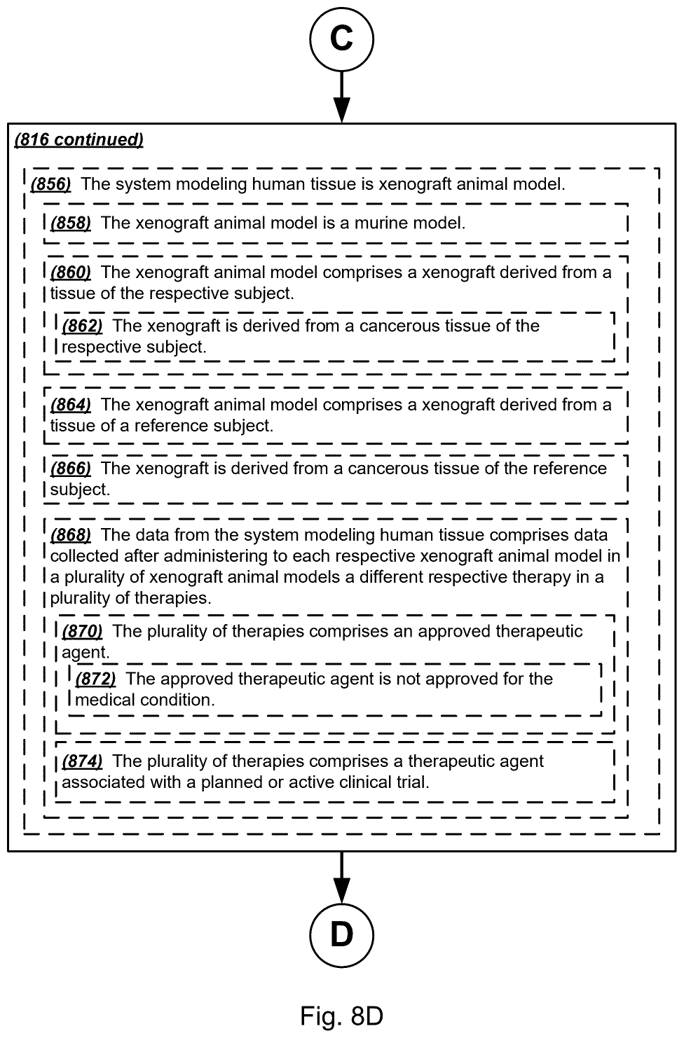

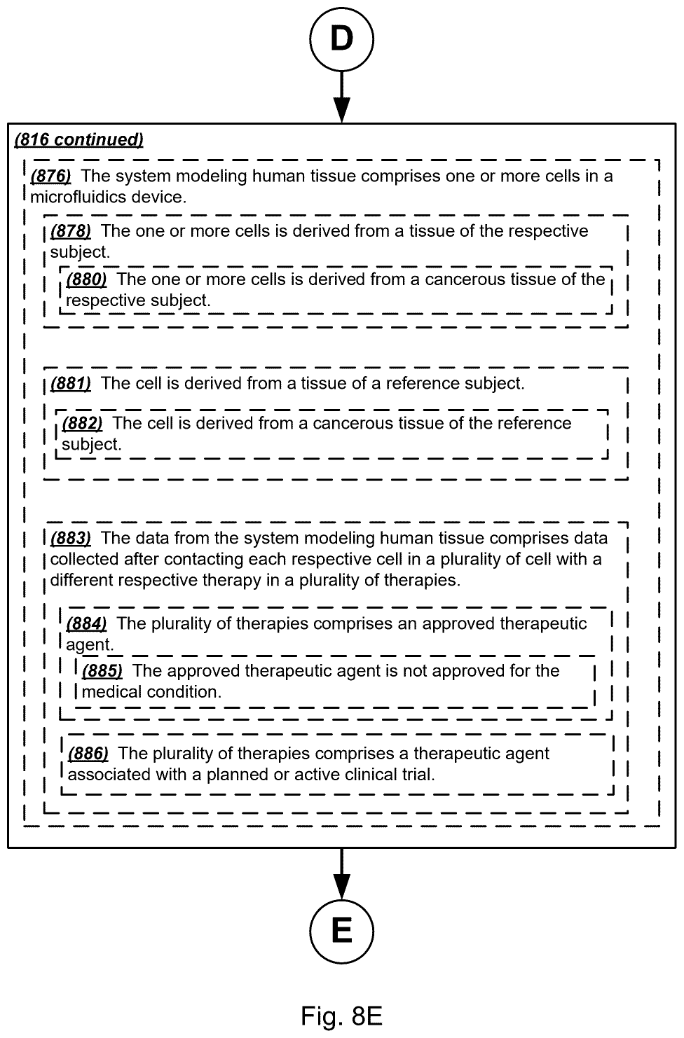

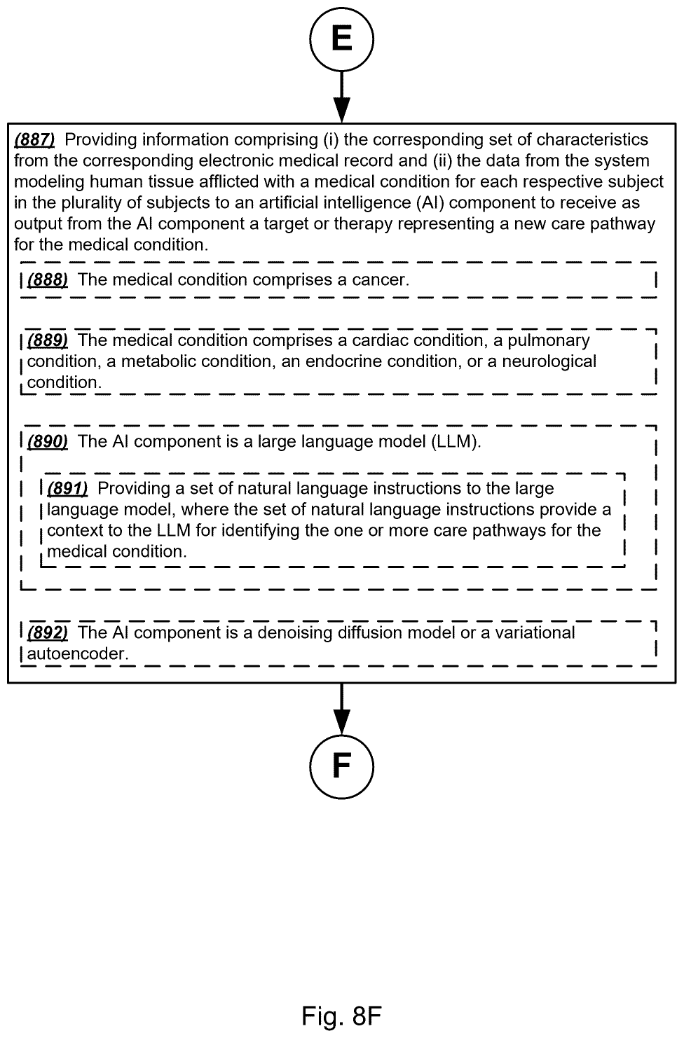

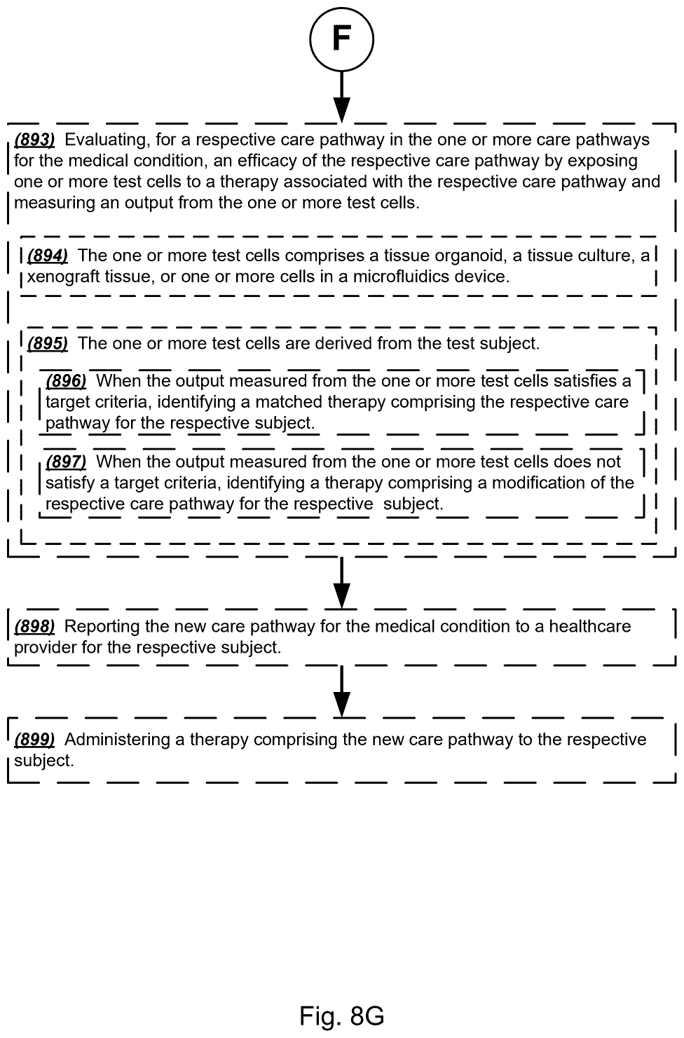

FIGS. 8A-8G collectively provide a flow chart of processes and features identifying a new care pathway for a medical condition, in accordance with some embodiments of the present disclosure.

The present disclosure provides a method 800 identifying a new care pathway for a medical condition. In some embodiments, all or a portion of method 800 is performed at a computer system having one or more processors and memory storing one or more programs for execution by the one or more processors.

Block 802. Referring to block 802, in some embodiments, retrieving, for each respective subject in a plurality of subjects, a corresponding set of characteristics of the respective subject from a corresponding electronic medical record for the respective subject. See also block 502 for further description of suitable characteristics and electronic medical records.

Block 804. Referring to block 804, in some embodiments, the set of characteristics is retrieved from the electronic medical record by retrieving structured data from the electronic medical record. See also block 504 for further description of structured data.

Block 806. Referring to block 806, in some embodiments, retrieving the set of characteristics from the electronic medical record comprises retrieving unstructured data from the electronic medical record. See also block 506 for further description of unstructured data.

Block 808. Referring to block 808, in some embodiments, retrieving the set of characteristics from the electronic medical record comprises evaluating at least three data types selected from structured electronic health record (EHR) data, unstructured EHR data, laboratory results, prescribed medications, and performed medical procedures. See also block 508 for further description of such combinations of data.

Block 810. Referring to block 810, in some embodiments, the laboratory results comprises histology data, medical imaging data, genomic sequencing data, transcriptomic data, proteomic data, phlebotomy data, vital signs, or anthropometric data. See also block 510 for further description of such data.

Blocks 812-814. Referring to block 812, in some embodiments, retrieving the set of characteristics from the electronic medical record comprises inputting all or a portion of the electronic medical record into a large language model (LLM). Referring to block 814, in some embodiments, further comprising providing a set of natural language instructions to the LLM, where the set of natural language instructions provide a context to the LLM to identify characteristics associated with the medical condition in the electronic medical record. See also block 812 and 814 for description of LLMs and the use of natural language instructions to provide context to LLMs.

Block 816. Referring to block 816, in some embodiments, retrieving the set of characteristics from the electronic medical record comprises retrieving information from a database populated from the electronic medical record. See also block 516 for further description of such databases.

Block 817. Referring to block 817, in some embodiments, retrieving data from a system modeling human tissue associated with a medical condition. See also block 517 for description of such systems modeling human tissue associated with a medical condition.

Block 818. Referring to block 818, in some embodiments, the system modeling human tissue is an organoid culture. An example method for culturing tumor organoids is described in U.S. Pat. No. 11,629,385, the content of which is incorporated herein by reference in its entirety. In some embodiments, an organoid, e.g., a tumor organoid, is co-cultured with one or more immune or effector cells. In some embodiments, the one or more immune or effector cells are derived from the same patient as is the organoid (e.g., the tumor organoid). Example methods for co-culturing organoids and immune or effector cells are described in U.S. Patent Application Publication No. 2023/0036156, the content of which is incorporated herein by reference in its entirety.

Blocks 820-822. Referring to block 820, in some embodiments, the organoid culture comprises organoids derived from a tissue of the test subject. Referring to block 822, in some embodiments, the organoid culture comprises tumor organoids derived from a cancerous tissue of the test subject. See blocks 520-522 for further description of such organoids.

Blocks 824. Referring to block 824, in some embodiments, the organoid culture comprises organoids derived from a tissue of a reference subject. In some embodiments, the organoids are derived not from a single individual but from a cohort of reference subjects, which may include healthy individuals selected to represent a normative biological baseline. In some embodiments the cohort of reference subjects is 2, 3, 4, 5, 6, 7, 8, 9, 10, 11, 12, 13, 14, 15, 16, 17, 18, 19, 20 or more different reference subjects. In some embodiments the cohort of reference subjects is 20, 30, 40, 50, 60, 70, 80, 90, 100, 110, 120, 130, 140, 150, 160, 170, 180, 190, or 200 different reference subjects. In some embodiments such reference subjects are healthy. In some embodiments the cohort subjects represent a broad array of different tissues, such as 5 or more, 10 or more, 15 or more, or 20 or more different tissues, meaning that for each such respective tissue, there is at least 1, 2, 3, 4, or 5 or more organoids derived from the respective tissue.

The tissue used to generate the organoids may be obtained from elective biopsies, surgical discards, or donor programs, and may include tissue from organs such as the colon, lung, liver, pancreas, kidney, prostate, or breast.

Organoids derived from healthy reference subjects enable systematic comparison between diseased and non-diseased tissues under identical experimental conditions. For example, when evaluating drug effects, such reference organoids provide a control group for determining whether observed cytotoxicity is tumor-specific or reflects general tissue toxicity. They may also help identify off-target effects, reveal differential pathway activation in healthy versus cancerous tissues, or serve as controls for molecular profiling assays such as RNA sequencing or proteomics. Moreover, organoids from a healthy cohort can be stratified by age, sex, ancestry, or other biological factors to assess population-level variability in baseline tissue response.

Block 826. Referring to block 826, in some embodiments, the organoid culture comprises tumor organoids derived from a cancerous tissue of the reference subject. See block 526 for further description of such organoids.

Block 828. Referring to block 828, in some embodiments, the data from the system modeling human tissue comprises data collected after contacting each respective organoid culture in a plurality of organoid cultures with a different respective therapy in a plurality of therapies. In some embodiments, the plurality of therapies includes any of the therapies, drug targets, and/or drug classes listed in block 528.

In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Block 830. Referring to block 830, in some embodiments, the plurality of therapies comprises an approved therapeutic agent. See block 530 for further description of such agents.

Block 832. Referring to block 832, in some embodiments, the approved therapeutic agent is not approved for the medical condition. See block 532 for further description of such situations.

Block 834 Referring to block 834, in some embodiments, the plurality of therapies comprises a therapeutic agent associated with a planned or active clinical trial. See block 534 for description of such clinical trials and the therapeutic agent associated with them.

Block 836. Referring to block 836, in some embodiments, the system modeling human tissue comprises a culture of one or more cell lines. See block 536 for a further description of such cell lines.

Block 838. Referring to block 838, in some embodiments, the cell line is derived from a tissue of the test subject. Test subject-derived cell lines may capture test subject-specific characteristics such as mutations, epigenetic features, or transcriptomic profiles, and may be used to validate findings observed in other model systems such as organoids or in silico predictions.

Block 840. Referring to block 840, in some embodiments, the cell line is derived from a cancerous tissue of the test subject. Tumor-derived cell lines may be used to evaluate responses to cytotoxic, targeted, or immunomodulatory agents, and may be particularly useful for longitudinal monitoring of therapeutic resistance or clonal evolution through successive rounds of treatment.

Block 842. Referring to block 842, in some embodiments, wherein the culture is a primary cell culture. These may be established directly from freshly resected or biopsied tissue without immortalization and maintained for a limited duration to preserve in vivo-like behavior. In some embodiments, combinations of primary and immortalized cell lines may be used in parallel to compare stable, scalable in vitro models with short-term, high-fidelity representations of patient biology.

In embodiments using multiple cell lines, data may be collected under standardized or perturbed conditions, with or without therapeutic agents, and subjected to comparative analysis across the full set of lines. This approach supports both individualized therapeutic modeling for the test subject and broader cohort-level or population-level inferences about treatment efficacy, drug resistance, or molecular mechanism of action.

Block 844. Referring to block 844, in some embodiments, the cell line is derived from a tissue of a reference subject. The reference subject may be a healthy donor or an individual with a well-characterized clinical and molecular background. In some embodiments, the cell lines are derived from a cohort of reference subjects, including individuals selected to reflect a range of demographic or genetic variables such as age, sex, ancestry, or known polymorphisms. The cell lines may be obtained from established biobanks, commercial cell repositories, or newly derived from donor tissue, including but not limited to skin, lung, gastrointestinal tract, kidney, liver, or other organ systems.

Reference-derived cell lines may serve multiple roles, such as providing a normative baseline for evaluating biological effects, controlling for assay variation, or enabling comparative modeling of disease versus non-disease states. For example, when evaluating the cytotoxic or molecular effects of a given therapy in a test subject's tumor-derived cell line, the inclusion of reference cell lines from healthy donors allows the identification of effects that are tumor-selective versus broadly cytotoxic. Moreover, data from reference cell lines may be used to calibrate high-throughput screens, normalize molecular readouts, or establish population-level thresholds for treatment response.

Block 846. Referring to block 846, in some embodiments, the cell line is derived from a cancerous tissue of the reference subject. See block 546 for description of cancerous tissues of reference subjects and cell lines derived from cancerous tissues of reference subjects.

Block 848. Referring to block 848, in some embodiments, the data from the system modeling human tissue comprises data collected after contacting each respective culture in a plurality of respective cultures with a different respective therapy in a plurality of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Blocks 850-854. Referring to blocks 850-854, in some embodiments, the plurality of therapies comprises any of the therapies disclosed in block 530 and/or block 532 and/or block 534.

Block 856. Referring to block 857, in some embodiments, the system modeling human tissue is one or more xenograft animal models. See block 586 for further description of xenograft animal models.

Block 858. Referring to block 858, in some embodiments, a xenograft animal model in the one or more xenograft animal models is a murine model.

Block 860. Referring to block 860, in some embodiments, the xenograft animal model comprises a xenograft derived from a tissue of the test subject. See block 860 for further description of a xenograft derived from a tissue of the test subject.

Block 862. Referring to block 862, in some embodiments, the xenograft is derived from a cancerous tissue of the test subject. See block 862 for further description of a xenograft derived from a cancerous tissue of a test subject.

Block 865. Referring to block 864, in some embodiments, the xenograft animal model comprises a xenograft derived from a tissue of a reference subject. See block 564 for further description of a xenograft animal model comprising a xenograft derived from a tissue of a reference subject.

Block 866. Referring to block 866, in some embodiments, the xenograft is derived from a cancerous tissue of the reference subject. See block 566 for further description of xenografts derived from cancerous tissue of reference subjects.

Block 868. Referring to block 868, in some embodiments, the data from the system modeling human tissue comprises data collected after administering to each respective xenograft animal model in a plurality of xenograft animal models a different respective therapy in a plurality of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Blocks 870-874. Referring to block 870, in some embodiments, the plurality of therapies comprises an approved therapeutic agent. Referring to block 872, in some embodiments, the approved therapeutic agent is not approved for the medical condition. Referring to block 874, in some embodiments, the plurality of therapies comprises a therapeutic agent associated with a planned or active clinical trial. In some embodiments, the plurality of therapies comprises any of the therapies disclosed in block 530 and/or block 532 and/or block 534.

Block 876. Referring to block 876, in some embodiments, the system modeling human tissue comprises one or more cells (e.g., examined) in a microfluidics device. In some embodiments, the cell is a single cell. In some embodiments, the cell is a tumor organoid. See block 576 for further disclosure of such system modeling.

Block 878. Referring to block 878, in some embodiments, the one or more cells are derived from a tissue of the test subject. Such cells may capture test subject-specific characteristics such as mutations, epigenetic features, or transcriptomic profiles, and may be used to validate findings observed in other model systems such as organoids or in silico predictions.

Block 880. Referring to block 880, in some embodiments, the one or more cells are derived from a cancerous tissue of the test subject. For instance, tumor cells may be used to evaluate responses to cytotoxic, targeted, or immunomodulatory agents, and may be particularly useful for longitudinal monitoring of therapeutic resistance or clonal evolution through successive rounds of treatment.

Block 881. Referring to block 881, in some embodiments, the one or more cells are derived from a tissue of a reference subject. The reference subject may be a healthy donor or an individual with a well-characterized clinical and molecular background. In some embodiments, the one or more cells are from a cohort of reference subjects, including individuals selected to reflect a range of demographic or genetic variables such as age, sex, ancestry, or known polymorphisms. The cells may be obtained from established biobanks, commercial cell repositories, or newly derived from donor tissue, including but not limited to skin, lung, gastrointestinal tract, kidney, liver, or other organ systems.

Reference-derived cells may serve multiple roles, such as providing a normative baseline for evaluating biological effects, controlling for assay variation, or enabling comparative modeling of disease versus non-disease states. For example, when evaluating the cytotoxic or molecular effects of a given therapy in a test subject's tumor-derived cell line, the inclusion of reference cells from healthy donors allows the identification of effects that are tumor-selective versus broadly cytotoxic. Moreover, data from reference cells may be used to calibrate high-throughput screens, normalize molecular readouts, or establish population-level thresholds for treatment response.

Block 882. Referring to block 882, in some embodiments, the one or more cells are derived from a cancerous tissue of the reference subject. See block 582 for further disclosure on such cells.

Block 883. Referring to block 883, in some embodiments, the data from the system modeling human tissue comprises data collected after contacting each respective cell in a plurality of cells with a different respective therapy in a plurality of therapies. In some embodiments, the plurality of therapies includes a small molecule therapy, an antibody therapy, an antibody-drug conjugate (ADC) therapy, a gene therapy, or the like. In some embodiments, the plurality of therapies is at least 2, at least 3, at least 4, at least 5, at least 10, at least 25, at least 100, at least 250, at least 500, at least 1000, at least 5000, at least 10,000, at least 50,000, at least 100,000, or more therapies.

Blocks 884-886. Referring to block 884, in some embodiments, the plurality of therapies comprises an approved therapeutic agent. Referring to block 885, in some embodiments, the approved therapeutic agent is not approved for the medical condition. Referring to block 886, in some embodiments, the plurality of therapies comprises a therapeutic agent associated with a planned or active clinical trial. In some embodiments, the plurality of therapies comprises any of the therapies disclosed in block 530 and/or block 532 and/or block 534.

Block 887. Referring to block 887, in some embodiments, information comprising (i) the corresponding set of characteristics from the corresponding electronic medical record and (ii) the data from the system modeling human tissue afflicted with a medical condition, for each respective subject in the plurality of subjects, is provided to an artificial intelligence (AI) component to receive as output from the AI component a target or therapy representing a new care pathway for the medical condition. Block 557 provides nonlimiting examples of how such information may be inputted into the AI component.

Block 888. Referring to block 888, in some embodiments, the medical condition comprises a cancer. Block 588 details example cancers.

Block 589. Referring to block 889, in some embodiments, the medical condition comprises a cardiac condition, a pulmonary condition, a metabolic condition, an endocrine condition, an immune condition, an autoimmune condition, a rare disease, psychiatric disorder, or a neurological condition. Examples of cardiac conditions may include: Atrial Fibrillation (AFib), aortic stenosis, cardiac amyloidosis, arrhythmia, stroke. Examples of pulmonary conditions may include asthma, COPD, chronic bronchitis, pneumonia, Pulmonary fibrosis, tuberculosis, emphysema, Bronchiectasis, Bronchiolitis, Bronchitis, Lung cancer, Pneumothorax or atelectasis, and Pulmonary edema. Examples of metabolic or endocrine conditions may include: diabetes, high blood pressure, hypertensions, hemochromatosis, Hypertriglyceridemia, Phenylketonuria, Porphyria, Gaucher Disease, Fabry Discase, Mitochondrial disease, Lysosomal storage disease, Hypothyroidism, Cushing's Syndrome, Hashimoto Thyroiditis, Hypercalcemia, osteoporosis, Pituitary disorders, Congenital adrenal hyperplasia, PCOS, adrenal insufficiency, Acromegaly. Examples of immune or autoimmune conditions include: Crohn's disease, celiac disease, ulcerative colitis, Graves' disease, Hashimoto's thyroiditis, Addison's disease, Multiple sclerosis (MS), chronic inflammatory demyelinating polyneuropathy (CIDP), Guillain-Barre syndrome, Rheumatoid arthritis (RA), psoriatic arthritis, Sjögren's syndrome, Dermatomyositis, psoriasis, allergy. Examples of a neurological or mental health condition or psychiatric disorder include: epilepsy, autism, neuromuscular disorders, attention deficit disorder (ADD), Alzheimer's Disease, Amyotrophic Lateral Sclerosis (ALS), Ataxia, Bell's Palsy, Multiple Sclerosis, headaches or migraines, stroke, hydrocephalus, Encephalitis, Muscular Dystrophy, Parkinson's Disease, Treatment Resistant Depression, Major Depressive Disorder, Bipolar Disorder, Schizophrenia.

Blocks 890-891. Referring to block 890, in some embodiments, the AI component is a large language model (LLM). Referring to block 891, in some embodiments, providing a set of natural language instructions to the large language model, wherein the set of natural language instructions provide a context to the LLM for identifying the one or more care pathways for the medical condition. Blocks 580 and 591 disclose LLMs and the use of natural language instructions to provide context to the input to LLMs.

Block 892. Referring to block 892, in some embodiments, the AI component is a denoising diffusion model or a variational autoencoder. Block 592 provides nonlimiting example details of denoising diffusion models and variational autoencoder in accordance with block 892.

Block 893. Referring to block 893, in some embodiments, the method also includes evaluating, for a respective care pathway in the one or more care pathways for the medical condition, an efficacy of the respective care pathway by exposing one or more test cells to a therapy associated with the respective care pathway and measuring an output from the one or more test cells. Block 593 provides example description of such evaluating.

Block 894. Referring to block 894, in some embodiments, the one or more test cells comprises a tissue organoid (see for instance blocks 520-534 on suitable organoids), a tissue culture, a xenograft tissue (see blocks 556-582 for suitable xenograph tissues), or one or more cells (e.g., examined) in a microfluidics device (see blocks 576-586 for such set ups).

Continuing to refer to block 894, in some embodiments, the one or more test cells used for evaluating therapeutic efficacy are selected from a range of biologically relevant models that replicate the tissue-specific and molecular characteristics of the medical condition. These may include tissue organoids, which are three-dimensional cell cultures derived from stem cells or primary tissues that self-organize to mimic the architecture and function of actual organs. Alternatively, the test cells may be in the form of 2D tissue cultures, which allow for rapid and scalable testing. In some embodiments, xenograft tissues, such as test subject derived xenografts (PDX) grown in immunocompromised mice, may be used to model in vivo drug responses. In other embodiments, the test cells may be housed in microfluidics-based devices or “organs-on-chips,” which enable dynamic perfusion and mechanical stimulation, simulating the physiological conditions of the human body and supporting high-resolution, real-time analysis of drug responses.

Block 895. Referring to block 895, in some embodiments, the one or more test cells used in the evaluation process are test subject specific, meaning they are derived directly from the test subject. These may include biopsy-derived tumor cells, circulating tumor cells (CTCs), reprogrammed induced pluripotent stem cells (iPSCs), or normal somatic cells transformed for modeling purposes. This ensures that the biological assay reflects the unique genetic, epigenetic, and microenvironmental context of the individual patient, thereby enabling a highly personalized and relevant evaluation of therapeutic efficacy.

Block 896. Referring to block 896, in some embodiments, when the output measured from the one or more test cells satisfies predefined criteria, such as significant reduction in cell viability, induction of apoptosis, or biomarker expression consistent with therapeutic response, then the associated therapy is designated a matched therapy.

Block 897. Referring to block 897, in some embodiments, when the measured output from the test cells fails to satisfy the target criteria, indicating insufficient therapeutic effect, toxicity, or resistance, the system dynamically adjusts the care pathway. This may involve identifying a modified therapy, such as a different drug, a combination regimen, a dose adjustment, or an alternative treatment modality altogether (e.g., shifting from chemotherapy to immunotherapy). This adaptive process ensures that the recommended care pathway is both evidence-informed and biologically validated, minimizing the risk of ineffective treatment.

Block 898. Referring to block 898, in some embodiments, the method also includes reporting the new care pathway for the medical condition to a healthcare provider for the respective subject. This reporting may take the form of a structured digital summary integrated into the electronic health record (EHR), a clinical decision support alert, or a narrative report containing relevant patient data, assay results, therapeutic rationale, and supporting evidence. This empowers the clinician with actionable insights grounded in both computational prediction and biological validation.

Block 899. Referring to block 899, in some embodiments, the method also includes administering therapy specified by the new care pathway to the respective subject. This may involve initiating a prescribed drug regimen, enrolling the test subject in a matched clinical trial, performing a surgical intervention, or applying any other medically appropriate action outlined in the new care pathway.

### Clinical Reports

In some embodiments, the methods described herein include generating a clinical report (e.g., a patient report), providing clinical support for personalized therapy as described above. In some embodiments, the report is provided to a patient, physician, medical personnel, or researcher in a digital copy (for example, a JSON object, a pdf file, or an image on a website or portal), a hard copy (for example, printed on paper or another tangible medium). A report object, such as a JSON object, can be used for further processing and/or display. For example, information from the report object can be used to prepare a clinical laboratory report for return to an ordering physician. In some embodiments, the report is presented as text, as audio (for example, recorded or streaming), as images, or in another format and/or any combination thereof.

In some embodiments, the report includes information related to the specific characteristics of the patient's biology, e.g., detected genetic variants, epigenetic abnormalities, associated oncogenic pathogenic infections, and/or pathology abnormalities. In some embodiments, other characteristics of a patient's sample and/or clinical records are also included in the report. For example, in some embodiments, the clinical report includes information on clinical variants, e.g., one or more of copy number variants (e.g., for actionable genes CCNEI, CD274 (PD-L1), EGFR, ERBB2 (HER2), MET, MYC, BRCA1, and/or BRCA2), fusions, translocations, and/or rearrangements (e.g., in actionable genes ALK, ROSI, RET, NTRK1, FGFR2, FGFR3, NTRK2 and/or NTRK3), pathogenic single nucleotide polymorphisms, insertion-deletions (e.g., somatic/tumor and/or germline/normal), therapy biomarkers, microsatellite instability status, and/or tumor mutational burden.

In some embodiments, one or more therapy, e.g., care pathway, identified for a subject using the methods and systems disclosed herein are provided in a clinical summary report. In some embodiments, a clinical report includes information about clinical trials for which the patient is eligible, therapies that are specific to the patient's biology, and/or possible therapeutic adverse effects associated with the specific characteristics of the patient's biology, e.g., the patient's genetic variations, epigenetic abnormalities, associated oncogenic pathogenic infections, and/or pathology abnormalities, or other characteristics of the patient's sample and/or clinical records. For example, in some embodiments, the clinical report includes such patient information and analysis metrics, e.g., cancer type and/or diagnosis, variant allele fraction, patient demographic and/or institution, matched therapies (e.g., FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

In some embodiments, the results included in the report, and/or any additional results are used to query a database of clinical data, for example, to determine whether there is a trend showing that a particular therapy was effective or ineffective in treating (e.g., slowing or halting cancer progression), and/or adverse effects of such treatments in other patients having the same or similar characteristics.

In some embodiments, the results are used to design cell-based studies of the patient's biology, e.g., tumor organoid experiments. For example, an organoid may be genetically engineered to have the same characteristics as the specimen and may be observed after exposure to a therapy to determine whether the therapy can reduce the growth rate of the organoid, and thus may be likely to reduce the growth rate of cancer in the patient associated with the specimen. Similarly, in some embodiments, the results are used to direct studies on tumor organoids derived directly from the patient. An example of such experimentation is described in U.S. Pat. No. 11,415,571, the content of which is hereby incorporated by reference, in its entirety, for all purposes.

### SPECIFIC EMBODIMENTS OF THE DISCLOSURE

In some aspects, the systems and methods disclosed herein may be used to support clinical decisions for personalized treatment of a disorder, e.g., a cancer, a cardiac condition, a pulmonary condition, a metabolic condition, an endocrine condition, an immune condition, an autoimmune condition, a rare disease, psychiatric disorder, a neurological condition, or other medical condition. Identified treatment modalities can be therapeutic drugs and/or assignment to one or more clinical trials. The therapies identified through use of the methods and systems described herein may constitute only a portion of a care pathway for a subject, e.g., they may be supplemented with additional therapies. Generally, current treatment guidelines for various cancers are maintained by various organizations, including the National Cancer Institute and Merck & Co., in the Merck Manual.

In some embodiments, the methods described herein further includes assigning therapy and/or administering therapy to the subject for a disorder, e.g., a cancer, a cardiac condition, a pulmonary condition, a metabolic condition, an endocrine condition, an immune condition, an autoimmune condition, a rare disease, psychiatric disorder, a neurological condition, or other medical condition, based on the identification of a care pathway through the methods and systems described herein. Assignment or administration of a therapy or a clinical trial to a subject is thus tailored for treatment of the disorder based on the individual biology of the patient.

### ADDITIONAL EMBODIMENTS

Another aspect of the present disclosure provides a computer system comprising one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

Although inventions have been particularly shown and described with reference to a preferred embodiment and various alternate embodiments, it will be understood by persons skilled in the relevant art that various changes in form and details can be made therein without departing from the spirit and scope of the invention.

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### ADDITIONAL CONSIDERATIONS

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter, in some embodiments, includes not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein, in some embodiments, are claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

1. A method for predicting one or more care pathway options for a medical condition in a subject, comprising:
at a computer system comprising a memory and a processor, the memory storing a plurality of instructions executable by the processor to perform a process comprising:
retrieving a set of characteristics of the subject from an electronic medical record for the subject,
retrieving data from a system modeling human tissue, and
providing information comprising the set of characteristics from the electronic medical record and the data from the system modeling human tissue to an artificial intelligence (AI) component to receive as output from the AI component one or more care pathways for the medical condition.

2-3. (canceled)

4. The method of claim 1, wherein retrieving the set of characteristics from the electronic medical record comprises evaluating at least three data types selected from:
structured electronic health record (EHR) data;
unstructured EHR data;
laboratory results;
prescribed medications; and
performed medical procedures.

5. The method of claim 4, wherein retrieving the set of characteristics from the electronic medical record comprises evaluating laboratory results, wherein the laboratory results comprise histology data, medical imaging data, genomic sequencing data, transcriptomic data, proteomic data, phlebotomy data, vital signs, or anthropometric data.

6. The method of claim 1, wherein retrieving the set of characteristics from the electronic medical record comprises inputting all or a portion of the electronic medical record into a large language model (LLM), the method further comprising providing a set of natural language instructions to the LLM, wherein the set of natural language instructions provide a context to the LLM to identify characteristics associated with the medical condition in the electronic medical record.

7-8. (canceled)

9. The method of, wherein the system modeling human tissue is an organoid culture, a culture of a cell line, a xenograft animal model, one or more cells in a microfluidics device, or any combination thereof.

10. The method of claim 1, wherein the system modeling human tissue comprises a plurality of organoids derived from a tissue of the subject, a plurality of tumor organoids derived from a cancerous tissue of the subject, a plurality of organoids derived from a tissue of at least one reference subject, or a plurality of tumor organoids derived from a cancerous tissue of each subject of the at least one reference subject.

11-13. (canceled)

14. The method of claim 10, wherein the data from the system modeling human tissue comprises data collected after contacting each respective organoid culture in the plurality of organoid cultures with a different respective therapy in a plurality of therapies.

15. The method of claim 14, wherein the plurality of therapies comprises an approved therapeutic agent.

16. The method of claim 15, wherein the approved therapeutic agent is not approved for the medical condition.

17. (canceled)

18. The method of claim 1, wherein the system modeling human tissue comprises a plurality of cultures of a cell line derived from a tissue of the subject, a plurality of cultures of a cell line derived from a cancerous tissue of the subject, a plurality of cultures that are of a primary cell culture, a plurality of cultures of a cell line derived from a tissue of at least one reference subject, or a plurality of cultures of a cell line derived from a cancerous tissue of each subject of the at least one reference subject.

19-23. (canceled)

24. The method claim 18, wherein the data from the system modeling human tissue comprises data collected after contacting each respective culture in the plurality of cultures with a different respective therapy in a plurality of therapies.

25-27. (canceled)

28. The method of claim 1, wherein the system modeling human tissue comprises a plurality of xenograft models, wherein the plurality of xenograft models is a plurality of murine models or wherein the plurality of xenograft models comprises a plurality of xenografts derived from a tissue of the subject or wherein the plurality of xenograft models is derived from a cancerous tissue of the subject, or wherein the plurality of xenograft models comprises a xenograft derived from a tissue of at least one reference subject, or wherein the plurality of xenograft models is derived from a cancerous tissue of each subject of the at least one reference subject.

29-33. (canceled)

34. The method of claim 28, wherein the data from the system modeling human tissue comprises data collected after administering to each respective xenograft animal model in the plurality of xenograft animal models a different respective therapy in a plurality of therapies.

35-37. (canceled)

38. The method of claim 1, wherein the system modeling human tissue comprises a plurality of cells in a microfluidics device, wherein the plurality of cells are derived from a tissue of the subject, wherein the plurality of cells are derived from a cancerous tissue of the subject, wherein the plurality of cells are derived from a tissue of at least one reference subject, or wherein the plurality of cells are derived from a tissue of each subject of the at least one reference subject.

39-42. (canceled)

43. The method of claim 38, wherein the data from the system modeling human tissue comprises data collected after contacting each respective cell in the plurality of cells with a different respective therapy in a plurality of therapies.

44-46. (canceled)

47. The method of claim 1, wherein the medical condition comprises a cancer, a cardiac condition, a pulmonary condition, a metabolic condition, an endocrine condition, an immune condition, an autoimmune condition, a rare disease, psychiatric disorder, or a neurological condition.

48. (canceled)

49. The method of claim 1, wherein the AI component is a large language model (LLM), the method further comprising providing a set of natural language instructions to the LLM, wherein the set of natural language instructions provide a context to the LLM for identifying the one or more care pathways for the medical condition.

50-51. (canceled)

52. The method according to claim 1, further comprising, for a respective care pathway in the one or more care pathways for the medical condition, evaluating an efficacy of the respective care pathway by exposing one or more test cells to a therapy associated with the respective care pathway and measuring an output from the one or more test cells, wherein the one or more test cells comprises a tissue organoid, a tissue culture, a xenograft tissue, or a cell in a microfluidics device, or wherein the one or more test cells are derived from the subject.

53-57. (canceled)

58. The method according to claim 1, further comprising administering a therapy comprising the respective care pathway to the subject.

59. A method for predicting care pathway options for a subject, comprising:
at a first computer system comprising a memory and a processor, the memory storing a plurality of instructions executable by the processor to perform a first process comprising:
providing information from an electronic medical record for the subject to a first artificial intelligence (AI) component to determine a set of therapies for a medical condition;
testing the set of therapies on a system modeling human tissue afflicted with the medical condition to receive modeling data as output from the testing; and
at a second computer system comprising a memory and a processor, the memory storing a plurality instructions executable by the processor to perform a second process comprising:
providing second information comprising the modeling data to a second AI component to receive as output from the AI component one or more care pathways for the medical condition.

60-110. (canceled)

111. A method for evaluating a medical condition in a subject, comprising:
at a first computer system comprising a memory and a processor, the memory storing a plurality of instructions executable by the processor to perform a first process comprising:
providing information from an electronic medical record for the subject to a model to receive, as output from the model, a set of tests for modeling a tissue;
performing the set of tests on a system modeling human tissue to receive modeling data as output from the testing; and
at a second computer system comprising a memory and a processor, the memory storing a plurality instructions executable by the processor to perform a second process comprising:
providing second information comprising the modeling data to an artificial intelligence (AI) component to receive as output from the AI component an analysis of the medical condition in the subject.

112-168. (canceled)

169. A method for identifying a new care pathway for a medical condition comprising:
at a computer system comprising a memory and a processor, the memory storing a plurality of instructions executable by the processor to perform a process comprising:
retrieving, for each respective subject in a plurality of subjects, a corresponding set of characteristics of the respective subject from a corresponding electronic medical record for the respective subject,
retrieving data from a system modeling human tissue associated with a medical condition,
providing information comprising i) the corresponding set of characteristics from the corresponding electronic medical record and ii) the data from the system modeling human tissue afflicted with a medical condition, for each respective subject in the plurality of subjects, to an artificial intelligence (AI) component to receive as output from the AI component a target or therapy representing a new care pathway for the medical condition.

170-228. (canceled)
